(19) United States
(12) Patent Application Publication
(10) Pub. No.: US 2019/0290656 A1
(43) Pub. Date: Sep. 26, 2019

(54) PT(IV) PRODRUGS

(71) Applicant: YISSUM RESEARCH DEVELOPMENT COMPANY OF THE HEBREW UNIVERSITY OF JERUSALEM LTD, Jerusalem (IL)

(72) Inventor: Dan Gibson, Jerusalem (IL)

(21) Appl. No.: 16/066,893

(22) PCT Filed: Dec. 29, 2016
(86) PCT No.: PCT/IL2016/051398

§ 371 Date: Jun. 28, 2018

Related U.S. Application Data
(60) Provisional application No. 62/272,741, filed on Dec. 30, 2015.

(51) Int. Cl. A61K 31/555; A61K 31/282; A61K 31/19; A61P 35/00

(52) U.S. Cl. CPC A61K 31/555; A61P 35/00; A61K 31/19; A61K 31/282

(57) ABSTRACT

The invention provides a novel class of Pt-based anticancer compounds exhibiting multiple anticancer activity.

### CROSS-REFERENCE TO RELATED APPLICATIONS

This application is a National Stage of International Application No. PCT/IL2016/051398, filed Dec. 29, 2016 which claims priority to U.S. Patent Application No. 62/272,741, filed Dec. 30, 2015, both of which are hereby incorporated by reference in their entireties.

### TECHNOLOGICAL FIELD

The invention concerns novel anticancer agents combining a platinum(IV) moiety with therapeutically active ligands for use as prodrugs for e.g., cancer treatment.

### BACKGROUND

Platinum anticancer agents are among the most widely used chemotherapeutic drugs and are administered in about 50% of all chemotherapeutic regimens. Two of the major drawbacks involved in their administration are the need for intravenous administration, which requires hospitalization, incurring significant costs, and the ability of tumors to develop resistance to these drugs.

One approach clinicians use for the treatment of unresponsive cancer patients is to use drug cocktails that act by different mechanisms.

Histone deacetylase inhibitors (HDACs) are emerging as a new class of anticancer drugs that can alter gene transcription and exert antitumor effects such as growth arrest, differentiation, apoptosis, and inhibition of tumour angiogenesis. In 2006, the FDA approved the first HDAC inhibitor—suberoylanilide hydroxamic acid (SAHA, Vorinostat), to treat the rare cutaneous T-cell lymphoma (CTCL). There are several clinical trials where HDAC inhibitors have been utilized in combination with platinum anticancer drugs. An ongoing trial (phase 2) entitled “Valproic Acid and Platinum-based Chemoradiation in Locally Advanced Head and Neck Squamous Cell Carcinoma” aimed to evaluate if the addition of valproic acid to standard platinum-based chemo-radiation as definitive treatment of locally advanced head and neck squamous cell carcinoma could improve treatment outcomes, such as response rate.

A Phase I Clinical Trial of Vorinostat in combination with Gemcitabine plus Platinum in patients with advanced Non-Small Cell Lung Cancer was completed in 2011.

Another approach to treating unresponsive cancer patients is to improve the therapeutic profiles of the anticancer drug.

Yang et al. described the synthesis of a complex of Pt(IV) prodrug with valproic acid, a specific histone deacetylase inhibitor, and its experiments in treatment of human carcinoma cell lines W.

### REFERENCES

[1] Yang J, Sun X, Mao W, Sui M, Tang J, Shen Y., Molecular Pharmaceutics, 9(10), 2793-2800 (2012).
[2] WO 2015/166498.

### SUMMARY OF THE INVENTION

The inventors of the present invention have developed a family of complex (conjugate) molecules comprising an anticancer platinum drug associated with one or more another therapeutically active anticancer drug, for the treatment of a variety of cancers.

The presence of more than one active moiety and the ability to permit simultaneous release, in cancer cells, of the active moieties, each acting via a different anticancer pathway permits eradication of cancer cells by acting on, or activating, more than one different cellular targets, increasing the chances of effective cancer cells eradication, including those resistant to anticancer drugs, and patient survivability.

Thus, the invention provides platinum based multifunctional compounds, each containing one or more platinum center and one or more therapeutically active anticancer moiety.

In one aspect the invention provides a compound comprising at least one platinum atom associated to one or more phenylbutyrate ligand, and optionally one or more therapeutically active anticancer moiety. The one or more therapeutically active anticancer moiety may be associated, bonded or coordinated to the platinum atom or to any one atom, group or moiety present in the compound.

In some embodiments, the compound of the invention is in the form of a prodrug, capable of releasing the phenylbutyrate ligand or any of the one or more optionally present therapeutically active anticancer moieties, thereby affecting anticancer activity.

In another aspect, there is provided a Pt-anticancer agent comprising one or more phenylbutyrate.

In some embodiments, the Pt-anticancer agent comprises at least two phenylbutyrate ligands; in other embodiments, the Pt-anticancer agent comprises at least three phenylbutyrate ligands, and yet in other embodiments, the Pt-anticancer agent comprises at least four phenylbutyrate ligands, such that in each of the aforesaid embodiments, the one or more optionally present therapeutically active anticancer moiety is not phenylbutyrate or a derivative thereof.

In some embodiments, the Pt-anticancer agent is associated to 2 or 3 or 5 or 6 phenylbutyrate ligands. Some embodiments relating to compounds of the invention are depicted below:

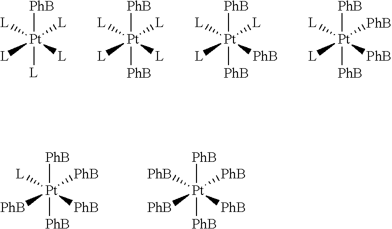

Typically, the phenylbutyrate ligand associated with the Pt atom is a monodentate ligand. Where the complex of the invention includes a single phenylbutyrate ligand, the complex may comprise in addition one or more polydentate ligand or one or more additional monodentate ligand, being different from phenylbutyrate. The “polydentate ligand”, being a ‘donor group’, is a ligand having more than one atom that can associate (or link, coordinate) directly to the Pt atom in a complex according to the invention; wherein a “monodentate” ligand forms a single bond with the metal atom.

In some embodiments, the complex has at least one monodentate ligand.

In some embodiments, the complex has at least one polydentate ligand. In some embodiments, the at least one polydentate ligand is a bidentate ligand. In some embodiments, the at least one polydentate ligand is a tridentate ligand. In some embodiments, the at least one polydentate ligand is a tetradentate ligand.

In some embodiments, the complex of the invention comprises two monodentate phenylbutyrate ligands.

The complex of the invention may be in any structural isomerization or stereoisomerization or optical isomers. In some embodiments, the complex is a cis isomer. In some embodiments, the complex is a trans isomer. In some embodiments, the complex is a mer-isomer. In some embodiments, the complex is a fac-isomer.

In some embodiments, the complex is in an octahedral geometry, wherein at least two of the ligands are in the axial positions of the octahedral complex. In some embodiments, the complex is in an octahedral geometry, wherein two ligands are phenyl butyrate ligands positioned in the axial positions of the octahedral complex. In some embodiments, the complex is in an octahedral geometry, wherein one of the ligands positioned in an axial position of the octahedral complex is phenyl butyrate.

In some embodiments, the complex of the invention comprises one or more phenylbutyrate ligand and at least one additional ligand selected from ligands designated herein L1 through L47:

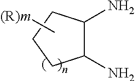

L1, wherein R is selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl, halogen, substituted or unsubstituted —NR₁R₂, substituted or unsubstituted —OR₃, substituted or unsubstituted —SR₄, substituted or unsubstituted —S(O)R₅, substituted or unsubstituted alkylene-COOH, substituted or unsubstituted ester, OH, —SH, and —NH, phenyl, hydroxyl; wherein each of R₁, R₂, R₃, R₄ and R₅ is independently selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl; wherein n being an integer between 0 and 2, wherein m being an integer between 0 and 5; and wherein the amine groups may be trans or cis to each other; and wherein the chiral carbons to which the amine groups are bonded may be R,R; R,S; or S,S; and wherein the ligand associates to the Pt via the amine moieties;

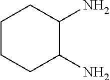

L2, wherein the amine groups may be trans or cis to each other; and wherein the chiral carbons to which the amine groups are bonded may be R,R; R,S; or S,S; and wherein the ligand associates to the Pt via the amine moieties;

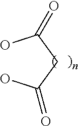

L3, wherein n being an integer between 0 and 5; wherein the ligand associates to the Pt via the oxygen atoms;

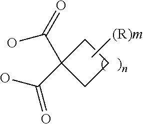

### —NH₂—R

L4, wherein R is selected from selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl, halogen, substituted or unsubstituted —NR₁R₂, substituted or unsubstituted —OR₃, substituted or unsubstituted —SR₄, substituted or unsubstituted —S(O)R₅, substituted or unsubstituted alkylene-COOH, substituted or unsubstituted ester, OH, —SH, and —NH, phenyl, hydroxyl; wherein each of R₁, R₂, R₃, R₄ and R₅ is independently selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl; and n being an integer between 0 and 2, and wherein m being an integer between 0 and 5; wherein the ligand associates to the Pt via the oxygen atoms;
L5, wherein R is null or selected from hydrogen, substituted or unsubstituted alkyl, substituted or unsubstituted cycloalkenyl, substituted or unsubstituted cycloalkynyl, substituted or unsubstituted cycloalkyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl, halogen, substituted or unsubstituted —NR₁R₂, substituted or unsubstituted —OR₃, substituted or unsubstituted —SR₄, substituted or unsubstituted —S(O)R₅, substituted or unsubstituted alkylene-COOH, substituted or unsubstituted ester, OH, —SH, and —NH, phenyl, hydroxyl; wherein each of R₁, R₂, R₃, R₄ and R₅ is independently selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl; wherein the ligand associates to the Pt via the amine moiety;

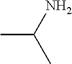

L6, wherein the ligand associates to the Pt via the amine moiety;

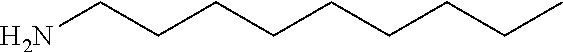

L7, wherein the ligand associates to the Pt via the amine moiety;

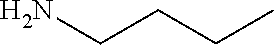

L8, wherein the ligand associates to the Pt via the amine moiety;

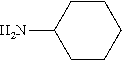

### —NHRR′

L10, wherein R and R′ are each independently selected from hydrogen, substituted or unsubstituted alkyl, substituted or unsubstituted cycloalkenyl, substituted or unsubstituted cycloalkynyl, substituted or unsubstituted cycloalkyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl, halogen, substituted or unsubstituted —NR₁R₂, substituted or unsubstituted —OR₃, substituted or unsubstituted —SR₄, substituted or unsubstituted —S(O)R₅, substituted or unsubstituted alkylene-COOH, substituted or unsubstituted ester, OH, —SH, and —NH, phenyl; wherein each of R₁, R₂, R₃, R₄ and R₅ is independently selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl; wherein the ligand associates to the Pt via the amine moiety;

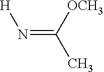

### —NC(O)OR

L11, wherein the ligand associates to the Pt via the amine moiety;
L12, wherein R is selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl, halogen, substituted or unsubstituted —NR₁R₂, substituted or unsubstituted —OR₃, substituted or unsubstituted —SR₄, substituted or unsubstituted —S(O)R₅, substituted or unsubstituted alkylene-COOH, substituted or unsubstituted ester, OH, —SH, and —NH, phenyl, hydroxyl, and wherein n being an integer between 0 and 5; wherein each of R₁, R₂, R₃, R₄ and R₅ is independently selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl; wherein the ligand associates to the Pt via the amine moieties;

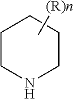

L13, wherein R is selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl, halogen, substituted or unsubstituted —NR₁R₂, substituted or unsubstituted —OR₃, substituted or unsubstituted —SR₄, substituted or unsubstituted —S(O)R₅, substituted or unsubstituted alkylene-COOH, substituted or unsubstituted ester, OH, —SH, and —NH, phenyl, hydroxyl; wherein each of R₁, R₂, R₃, R₄ and R₅ is independently selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl; and wherein n being an integer between 0 and 5; wherein the ligand associates to the Pt via the amine moieties;

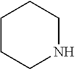

L14, wherein the ligand associates to the Pt via the amine moiety;

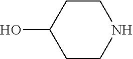

L15, wherein the ligand associates to the Pt via the amine moiety;

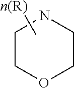

L16, wherein R is selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl, halogen, substituted or unsubstituted —NR₁R₂, substituted or unsubstituted —OR₃, substituted or unsubstituted —SR₄, substituted or unsubstituted —S(O)R₅, substituted or unsubstituted alkylene-COOH, substituted or unsubstituted ester, OH, —SH, and —NH, phenyl, hydroxyl; wherein each of R₁, R₂, R₃, R₄ and R₅ is independently selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl; and wherein n being an integer between 0 and 5; wherein the ligand associates to the Pt via the amine moieties;

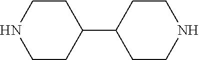 [SMILES: C1CC(C2CCNCC2)CCN1]

L17, wherein the ligand associates to the Pt via the amine moiety;

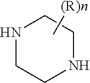

L18, wherein R is selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl, halogen, substituted or unsubstituted —NR₁R₂, substituted or unsubstituted —OR₃, substituted or unsubstituted —SR₄, substituted or unsubstituted —S(O)R₅, substituted or unsubstituted alkylene-COOH, substituted or unsubstituted ester, OH, —SH, and —NH, phenyl, hydroxyl; wherein each of R₁, R₂, R₃, R₄ and R₅ is independently selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl; and wherein n being an integer between 0 and 5; wherein the ligand associates to the Pt via the amine moieties;

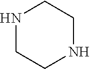

L19, wherein the ligand associates to the Pt via the amine moiety;

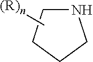

L20, wherein R is selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl, halogen, substituted or unsubstituted —NR₁R₂, substituted or unsubstituted —OR₃, substituted or unsubstituted —SR₄, substituted or unsubstituted —S(O)R₅, substituted or unsubstituted alkylene-COOH, substituted or unsubstituted ester, OH, —SH, and —NH, phenyl, hydroxyl; wherein each of R₁, R₂, R₃, R₄ and R₅ is independently selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl; and wherein n being an integer between 0 and 5; wherein the ligand associates to the Pt via the amine moieties;

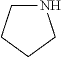

L21, wherein the ligand associates to the Pt via the amine moiety;

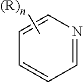

L22, wherein R is selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl, halogen, substituted or unsubstituted —NR₁R₂, substituted or unsubstituted —OR₃, substituted or unsubstituted —SR₄, substituted or unsubstituted —S(O)R₅, substituted or unsubstituted alkylene-COOH, substituted or unsubstituted ester, OH, —SH, and —NH, phenyl, hydroxyl; wherein each of R₁, R₂, R₃, R₄ and R₅ is independently selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl; and wherein n being an integer between 0 and 5; wherein the ligand associates to the Pt via the amine moieties;

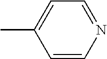

L23, wherein the ligand associates to the Pt via the amine moiety;

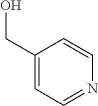

L24, wherein the ligand associates to the Pt via the amine moiety;

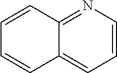

L25, wherein the ligand associates to the Pt via the amine moiety;

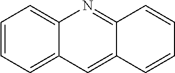 [SMILES: c1ccc2nc3ccccc3cc2c1]

L26, wherein the ligand associates to the Pt via the amine moiety;

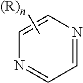

L27, wherein R is selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl, halogen, substituted or unsubstituted —NR₁R₂, substituted or unsubstituted —OR₃, substituted or unsubstituted —SR₄, substituted or unsubstituted —S(O)R₅, substituted or unsubstituted alkylene-COOH, substituted or unsubstituted ester, OH, —SH, and —NH, phenyl, hydroxyl; wherein each of R₁, R₂, R₃, R₄ and R₅ is independently selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl; and wherein n being an integer between 0 and 5; wherein the ligand associates to the Pt via the amine moieties;

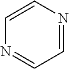

L28, wherein the ligand associates to the Pt via the amine moiety;

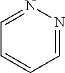

L29, wherein the ligand associates to the Pt via the amine moiety;

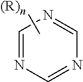

L30, wherein R is selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl, halogen, substituted or unsubstituted —NR₁R₂, substituted or unsubstituted —OR₃, substituted or unsubstituted —SR₄, substituted or unsubstituted —S(O)R₅, substituted or unsubstituted alkylene-COOH, substituted or unsubstituted ester, OH, —SH, and —NH, phenyl, hydroxyl; wherein each of R₁, R₂, R₃, R₄ and R₅ is independently selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl; and wherein n being an integer between 0 and 5; wherein the ligand associates to the Pt via the amine moieties;

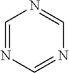

L31, wherein the ligand associates to the Pt via the amine moiety;

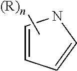

L32, wherein R is selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl, halogen, substituted or unsubstituted —NR₁R₂, substituted or unsubstituted —OR₃, substituted or unsubstituted —SR₄, substituted or unsubstituted —S(O)R₅, substituted or unsubstituted alkylene-COOH, substituted or unsubstituted ester, OH, —SH, and —NH, phenyl, hydroxyl; wherein each of R₁, R₂, R₃, R₄ and R₅ is independently selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl; and wherein n being an integer between 0 and 5; wherein the ligand associates to the Pt via the amine moieties;

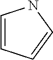

L33, wherein the ligand associates to the Pt via the amine moiety;

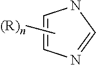

L34, wherein R is selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl, halogen, substituted or unsubstituted —NR₁R₂, substituted or unsubstituted —OR₃, substituted or unsubstituted —SR₄, substituted or unsubstituted —S(O)R₅, substituted or unsubstituted alkylene-COOH, substituted or unsubstituted ester, OH, —SH, and —NH, phenyl, hydroxyl; wherein each of R₁, R₂, R₃, R₄ and R₅ is independently selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl; and wherein n being an integer between 0 and 5; wherein the ligand associates to the Pt via the amine moieties;

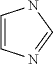

L35, wherein the ligand associates to the Pt via the amine moiety;

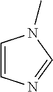

L36, wherein the ligand associates to the Pt via the amine moiety;

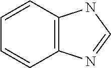

L37, wherein the ligand associates to the Pt via the amine moiety;

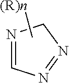

L38, wherein R is selected from hydrogen, substituted or unsubstituted alkyl, substituted or unsubstituted cycloalkyl, substituted or unsubstituted cycloalkenyl, substituted or heterocyclyl, substituted or unsubstituted aryl, substituted or unsubstituted heteroaryl, substituted or unsubstituted heterocyclyl, substituted or unsubstituted alkenyl, substituted or unsubstituted alkynyl, halogen, substituted or unsubstituted —NR₁R₂, substituted or unsubstituted —OR₃, substituted or unsubstituted —SR₄, substituted or unsubstituted —S(O)R₅, substituted or unsubstituted alkylene-COOH, substituted or unsubstituted ester, OH, —SH, and —NH, phenyl, hydroxyl; wherein each of R₁, R₂, R₃, R₄ and R₅ is independently selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl; and wherein n being an integer between 0 and 5; wherein the ligand associates to the Pt via the amine moieties;

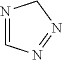

L39, wherein the ligand associates to the Pt via the amine moiety;

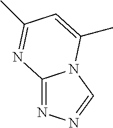

L40, wherein the ligand associates to the Pt via the amine moiety;

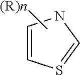

L41, wherein R is selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl, halogen, substituted or unsubstituted —NR₁R₂, substituted or unsubstituted —OR₃, substituted or unsubstituted —SR₄, substituted or unsubstituted —S(O)R₅, substituted or unsubstituted alkylene-COOH, substituted or unsubstituted ester, OH, —SH, and —NH, phenyl, hydroxyl; wherein each of R₁, R₂, R₃, R₄ and R₅ is independently selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl; and wherein n being an integer between 0 and 5; wherein the ligand associates to the Pt via the amine moieties;

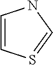

L43, wherein the ligand associates to the Pt via the amine moiety;

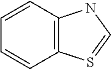

### —S(O)RR′

L43, wherein the ligand associates to the Pt via the amine moiety;
L44, wherein R and R′ are each independently selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl, halogen, substituted or unsubstituted —NR₁R₂, substituted or unsubstituted —OR₃, substituted or unsubstituted —SR₄, substituted or unsubstituted —S(O)R₅, substituted or unsubstituted alkylene-COOH, substituted or unsubstituted ester, OH, —SH, and —NH, phenyl, hydroxyl; wherein each of R₁, R₂, R₃, R₄ and R₅ is independently selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl; and wherein n being an integer between 0 and 5; wherein the ligand associates to the Pt via the sulfinyl moieties (S atom);

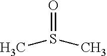

L45, wherein the ligand associates to the Pt via the sulfinyl moiety (S atom);

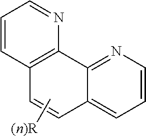

L46, wherein R is selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl, halogen, substituted or unsubstituted —NR₁R₂, substituted or unsubstituted —OR₃, substituted or unsubstituted —SR₄, substituted or unsubstituted —S(O)R₅, substituted or unsubstituted alkylene-COOH, substituted or unsubstituted ester, OH, —SH, and —NH, phenyl, hydroxyl; wherein each of R₁, R₂, R₃, R₄ and R₅ is independently selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl; and wherein n being an integer between 0 and 5; wherein the ligand associates to the Pt via the amine atoms;

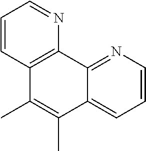 [SMILES: Cc1c(C)c2cccnc2c2ncccc12]

L47, wherein the ligand associates to the Pt via the amine atoms.

In each of the above ligands, the various substituting groups or radicals are selected as defined herein.

In some embodiments, at least one of the ligands is halide (Cl, Br, I or F). In some embodiments, at least one of the ligands is Cl.

In some embodiments, at least one of the ligands is an amine selected from ammonia, a primary amine, a secondary amine, a non-planar heterocyclic aliphatic amine or a heterocyclic aromatic amine. In some embodiments, at least one of the ligands is —NH₃. In some embodiments, at least one of the ligands is —NH₂.

In some embodiments, at least one of the ligands is a primary amine Non-limiting examples of a primary amine are methylamine, ethylamine, n-propylamine, isopropylamine, n-butylamine, n-hexylamine, n-heptylamine or n-nonylamine.

In some embodiments, at least one of the ligands is a secondary amine. Non limiting examples of a secondary amine are dimethylamine, diethylamine, dipropylamine, dibutylamine.

In some embodiments, at least one of the ligands is a non-planar heterocyclic aliphatic amine Non-limiting examples of a non-planar heterocyclic aliphatic amine are piperazine, 2-methylpiperazine, piperadine, 2-, 3- or 4-hydroxypiperidine, 4-piperidino-piperidine, pyrrolidine, 4-(2-hydroxyethyl)piperazine or 3-aminopyrrolidine.

In some embodiments, at least one of the ligands is a heterocyclic aromatic amine Non-limiting examples of a heterocyclic aromatic amine are pyridine, 2-, 3-, or 4-aminopyridine, 2-, 3-, or 4-picoline, quinoline, 3-, or 4-aminoquinoline, thiazole, imidazole, 3-pyrroline, pyrazine, 2-methylpyrazine, 4-aminooquinaldine, pyridazine, 1,10-phenanthroline and 5,6-dimethyl-1,10-phenanthroline.

In some embodiments, at least one of the ligands is selected from ligands herein designated L48 through L63:

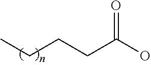

L48, wherein n is an integer between 0 and 6; the ligand associates to the Pt via the oxygen atom(s);
L49, wherein each of n and m independently is an integer between 0 and 6; the ligand associates to the Pt via the oxygen atom(s);

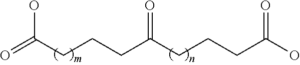

L50, wherein each of n and m independently is an integer between 0 and 6; the ligand associates to the Pt via the oxygen atom(s) and/or the nitrogen atom(s);

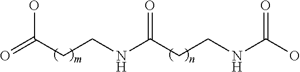

L51, wherein each of n and m independently is an integer between 0 and 6; the ligand associates to the Pt via the oxygen atom(s) and/or the nitrogen atom;

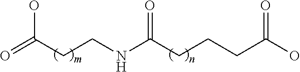

L52, wherein each of n and m independently is an integer between 0 and 6; the ligand associates to the Pt via the oxygen atom(s) and/or the nitrogen atom(s);

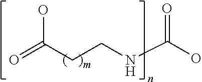

L53, the ligand associates to the Pt via the oxygen atom(s);

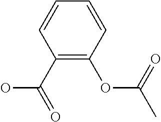

L54, the ligand associates to the Pt via the oxygen atom(s).

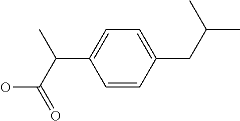

### —ROH

L55, wherein R is selected from substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl, halogen, substituted or unsubstituted —NR₁R₂, substituted or unsubstituted —OR₃, substituted or unsubstituted —SR₄, substituted or unsubstituted —S(O)R₅, substituted or unsubstituted alkylene-COOH, substituted or unsubstituted ester, OH, —SH, and —NH, phenyl, hydroxyl; wherein each of R₁, R₂, R₃, R₄ and R₅ is independently selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl; and wherein n being an integer between 0 and 5; wherein the ligand associates to the Pt via the hydroxide moiety;

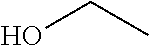

### —RC(O)OH

L56;
L57, wherein R is selected from substituted or unsubstituted alkyl, substituted or unsubstituted cycloalkenyl, substituted or unsubstituted cycloalkynyl, substituted or unsubstituted cycloalkyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl, halogen, substituted or unsubstituted —NR₁R₂, substituted or unsubstituted —OR₃, substituted or unsubstituted —SR₄, substituted or unsubstituted —S(O)R₅, substituted or unsubstituted alkylene-COOH, substituted or unsubstituted ester, OH, —SH, and —NH, hydroxyl group; wherein each of R₁, R₂, R₃, R₄ and R₅ is independently selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl; and wherein the ligand associates to the Pt via the oxygen atoms;

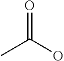

L58;

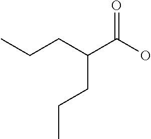

L59;

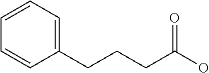 [SMILES: O=C(O)CCCc1ccccc1]

L60,

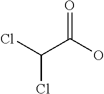

L62

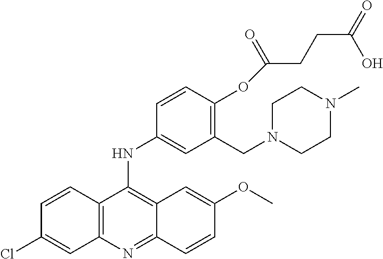 [SMILES: COc1ccc2nc3cc(Cl)ccc3c(Nc3ccc(OC(=O)CCC(=O)O)c(CN4CCN(C)CC4)c3)c2c1]

L62

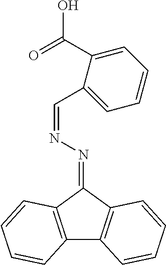 [SMILES: O=C(O)c1ccccc1/C=N\N=C1c2ccccc2-c2ccccc21]

L63.

In some embodiments, in compounds of the invention, each of the ligands is different from the other.

In some embodiments, in compounds of the invention, at least two of the ligands are identical.

The platinum atom may be selected from platinum(IV).

As stated herein, compounds of the invention may comprise one or more phenylbutyrate ligand, and optionally one or more moiety or ligand that is a therapeutically active anticancer moiety or ligand. The therapeutically active anticancer moiety or ligand is one that affects, modulates, inhibits or enhances at least one cancer-related pathway. The therapeutically active anticancer moiety or ligand is thus selected amongst:

Pyruvate dehydrogenase kinase (PDK) inhibitors;

Histone deacetylase (HDAC) inhibitors;

DNA methylation enhancers;

COX inhibitors;

PARP inhibitors;

DNA repair inhibitors; and

Cancer targeting moieties.

As known in the art, “pyruvate dehydrogenase kinase (PDK) inhibitors” are ligands or moieties which may be directly associated to the metal center in compounds of the invention or to any one atom, group or moiety, that are known to inactivate enzyme pyruvate dehydrogenase by phosphorylating it using ATP.

“Histone deacetylase (HDAC) inhibitors” are ligands or moieties which may be directly associated to the metal center in compounds of the invention or to any one atom, group or moiety, that are known to inhibit histone deacetylase.

“DNA methylation enhancers” are ligands or moieties which may be directly associated to the metal center in compounds of the invention or to any one atom, group or moiety, that are known to directly or indirectly cause methylation of a DNA molecule, thereby changing or modifying or affecting DNA activity without substantially changing the DNA sequence.

As known, “COX inhibitors” are ligands or moieties which may be directly associated to the metal center in compounds of the invention or to any one atom, group or moiety, that are known to directly or indirectly inhibit activity of Cyclooxygenase (COX), or prostaglandin-endoperoxide synthase (PTGS).

“PARP inhibitors” are ligands or moieties which may be directly associated to the metal center in compounds of the invention or to any one atom, group or moiety, that are known to inhibit function of poly ADP ribose polymerase (PARP).

“DNA repair inhibitors” are ligands or moieties which may be directly associated to the metal center in compounds of the invention or to any one atom, group or moiety, that are known to inhibit or interrupt one or more DNA repair mechanisms.

Cancer targeting moieties are typically tumor-cell-specific small-molecule ligand or moieties capable of targeting a therapeutic agent, e.g., a compound of the invention, to tumor cells.

In some embodiments, the PDK inhibitors may be dichloro acetate (DCA), pyrazols described in U.S. Patent Application No. 2003/236294, hereby incorporated by reference in its entirety, pyrimadine derivatives described in U.S. Application No. 2004/186118, hereby incorporated by reference in its entirety, pyrrolo[3,4-c]pyrazoles described in U.S. Application No. 2005/101594, hereby incorporated by reference in its entirety, and other inhibitors as disclosed in U.S. Application No. 2014/0005127, hereby incorporated by reference in its entirety.

In some embodiments, the PARP inhibitors may be those described in U.S. Pat. Nos. 8,623,872, 5,177,075, European Patent No. 103,6073 and U.S. Pat. No. 6,635,642 each being incorporated by reference in its entirety.

In some embodiments, the targeting moieties are selected amongst acids such as folic acid (folate), and materials such as those disclosed in U.S. Application No. US 20090061010, herein incorporated by reference.

In some embodiments, the DNA repair inhibitors are selected amongst nitrogen mustards and derivatives thereof, reported in Future Med. Chem. 2012; 4(9): 1093-1111 and in Front Pharmacol. 2013; 4: 5; and others.

In some embodiments, the COX inhibitors may be selected amongst NSAIDs such as ibuprofen, sulindac, celecoxib and aspirin.

In some embodiments, the DNA methylation enhancers may be selected from octanoate and octanoate derivatives, folate and others.

In some embodiments, the HDAC inhibitors may be phenyl butyrate, valproic acid and derivatives thereof, romidepsin, belinostat, panobinostat and vorinostat.

In some embodiments, the therapeutically active moieties or ligands are selected amongst ligands herein designated L48 (in some embodiments, n=4, 5, 6), L53, L54, L59, L60, L61, L62, L63.

In some embodiments, the therapeutically active moieties or ligands, one or more, may be associated or bonded to the metal atom, or to any one atom in a compound of the invention, directly through a native atom or bond present on the therapeutically active moiety or ligand, or through a linker moiety such as —O—C(═O)—, wherein one end of the linker moiety is bonded to the therapeutically active moiety or ligand and another is bonded to the compound (directly to the metal atom or to an atom of the compound).

As stated above, in a compound according to the invention, the platinum atom is associated to one or more phenylbutyrate ligand and at least one additional ligand, e.g., therapeutically active anticancer moiety or ligand. The term “associate”, or any lingual variation thereof, refers to any chemical or physical bond (linkage), such as covalent, ionic, Van der Walls or coordinative which holds the Pt atom and at least one of the ligand atoms together. Typically, the platinum atom is associated to the ligand(s) via coordinative bond(s).

In further embodiments, at least one ligand is bound to the platinum atom via at least one heteroatom selected from nitrogen, oxygen and sulfur. In some embodiments, some of the bonds between the metal atom and the heteroatoms are covalent and some of the bonds are coordinative bonds.

In some embodiments, the covalent bonds to the metal atom are via oxygen or sulfur atoms. In some embodiments, the coordinative bonds to the metal atoms are via nitrogen or sulfur atoms.

In another aspect the invention provides a compound of Formula (I):

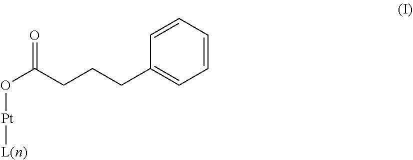

wherein

L is a ligand moiety selected from substituted or unsubstituted alkyl, substituted or unsubstituted alkenyl, substituted or unsubstituted alkynyl, substituted or unsubstituted cycloalkyl, substituted or unsubstituted cycloalkenyl, substituted or unsubstituted cycloalkynyl, substituted or unsubstituted aryl, substituted or unsubstituted heteroaryl, substituted or unsubstituted heterocyclyl, halogen, substituted or unsubstituted —NR₁R₂, substituted or unsubstituted —OR₃, substituted or unsubstituted —SR₄, substituted or unsubstituted —S(O)R₅, substituted or unsubstituted alkylene-COOH, substituted or unsubstituted ester, —OH, —SH, and —NH;

wherein each of said R₁, R₂, R₃, R₄ and R₅, independently of the other, is selected from substituted or unsubstituted alkyl, substituted or unsubstituted alkenyl, substituted or unsubstituted alkynyl, substituted or unsubstituted cycloalkyl, substituted or unsubstituted cycloalkenyl, substituted or unsubstituted cycloalkynyl, substituted or unsubstituted aryl, substituted or unsubstituted heteroaryl, substituted or unsubstituted heterocyclyl;

and wherein

n is the number of ligand moieties, being 1, 2, 3, 4, or 5.

As used herein, “alkyl”, “alkenyl” and “alkynyl” carbon chains, if not specified, contain from 1 to 20 carbons, or 1 or 2 to 16 carbons, and are straight or branched. In some embodiments, the carbon chain contains 1 to 10 carbon atoms. In some embodiments, the carbon chain contains 1 to 6 carbon atoms. In some embodiments, the carbon chain contains 2 to 6 carbon atoms. Alkenyl carbon chains of from 2 to 20 carbons, in certain embodiments, contain 1 to 8 double bonds and alkenyl carbon chains of 2 to 16 carbons, in certain embodiments, contain 1 to 5 double bonds. Alkynyl carbon chains of from 2 to 20 carbons, in certain embodiments, contain 1 to 8 triple bonds, in yet other embodiments, contain 1 to 5.

Exemplary alkyl, alkenyl and alkynyl groups herein include, but are not limited to, methyl, ethyl, propyl, isopropyl, isobutyl, n-butyl, sec-butyl, tert-butyl, isohexyl, allyl (propenyl) and propargyl (propynyl).

As used herein, “cycloalkyl” refers to a saturated mono- or multi-cyclic ring system, in certain embodiments of 3 to 10 carbon atoms, in other embodiments of 3 to 6 carbon atoms; cycloalkenyl and cycloalkynyl refer to mono- or multicyclic ring systems that respectively include at least one double bond and at least one triple bond. Cycloalkenyl and cycloalkynyl groups may, in certain embodiments, contain 3 to 10 carbon atoms, with cycloalkenyl groups, in further embodiments, containing 4 to 7 carbon atoms and cycloalkynyl groups, in further embodiments, containing 8 to 10 carbon atoms. The ring systems of the cycloalkyl, cycloalkenyl and cycloalkynyl groups may be composed of one ring or two or more rings which may be joined together in a fused, bridged or spiro-connected fashion.

As used herein, “aryl” refers to aromatic monocyclic or multicyclic groups containing from 6 to 10 carbon atoms. Aryl groups include, but are not limited to groups such as unsubstituted or substituted fluorenyl, unsubstituted or substituted phenyl, and unsubstituted or substituted naphthyl.

As used herein, “heteroaryl” refers to a monocyclic or multicyclic aromatic ring system, in certain embodiments, of about 5 to about 15 members where one or more, in one embodiment 1 to 3, of the atoms in the ring system is a heteroatom, that is, an element other than carbon, including but not limited to, nitrogen, oxygen or sulfur. The heteroaryl group may be optionally fused to a benzene ring. Heteroaryl groups include, but are not limited to, furyl, imidazolyl, pyrimidinyl, tetrazolyl, thienyl, pyridyl, pyrrolyl, thiazolyl, isothiazolyl, oxazolyl, isoxazolyl, triazolyl, quinolinyl and isoquinolinyl,

As used herein, “heterocyclyl” refers to a saturated mono- or multi-cyclic ring system, in one embodiment of 3 to 10 members, in another embodiment of 4 to 7 members, in a further embodiment of 5 to 6 members, where one or more, in certain embodiments, 1 to 3, of the atoms in the ring system is a heteroatom, that is, an element other than carbon, including but not limited to, nitrogen, oxygen or sulfur. In embodiments where the heteroatom(s) is nitrogen, the nitrogen is optionally substituted with alkyl, alkenyl, alkynyl, aryl, heteroaryl, aralkyl, heteroaralkyl, cycloalkyl, heterocyclyl, cycloalkylalkyl, heterocyclylalkyl, acyl, guanidine, or the nitrogen may be quaternized to form an ammonium group where the substituents are selected as above.

As used herein, “halogen” or “halide” refers to F, Cl, Br or I.

As used herein, “—NR₁R₂” refers to an amine group wherein R₁ and R₂ are independently selected from hydrogen, alkyl, alkenyl, alkenyl, cycloalkyl, cycloalkenyl, cycloalkynyl, aryl, heteroaryl, heterocyclyl, halogen, —C(O)NR₆R₇, sulfinyl, ester, carbonyl.

In some embodiments, amine group is selected from a primary amine (wherein each of R₁ and R₂ is —H), a secondary amine (wherein one of R₁ and R₂ is a —C₁-C₆alkyl) or a tertiary amine (wherein each of R₁ and R₂ is a —C₁-C₆alkyl, R₁ and R₂ need not be the same). In some embodiments, the —NR₁R₂ may represent a quaternary amine, wherein the N atom is further protonated or alkylated to a charged state, forming a salt with, e.g., at least one pharmaceutically acceptable counter-ion.

In some embodiments, R₁ and R₂ in —NR₁R₂ form a cyclic structure with the N atom they are bonded to; the cyclic amine having between 3 and 6 atoms in the hetero-ring structure. In some embodiments, the hetero-ring comprises, apart from the N atom, one or more additional heteroatoms selected from N, O and S. In further embodiments, the hetero-ring comprises a single heteroatom (the N atom of the —NR₁R₂ group) with the remaining atoms being carbon atoms.

As used herein, —C(O)NR₆R₇ wherein R₃ and R₄ are independently selected from hydrogen, alkyl, alkenyl, alkenyl, cycloalkyl, cycloalkenyl, cycloalkynyl, aryl, heteroaryl, heterocyclyl, halogen, —NR₁R₂, sulfinyl, ester, carbonyl, —OH, —SH and —NH.

As used herein, “—OR₃” refers to an hydroxyl group wherein R₃ is selected from hydrogen, alkyl, alkenyl, alkenyl, cycloalkyl, cycloalkenyl, cycloalkynyl, aryl, heteroaryl, heterocyclyl, halogen, sulfinyl, ester, carbonyl.

As used herein, “—SR₄” refers to an thiol group wherein R₄ is selected from hydrogen, alkyl, alkenyl, alkenyl, cycloalkyl, cycloalkenyl, cycloalkynyl, aryl, heteroaryl, heterocyclyl, halogen, sulfinyl, ester, carbonyl.

As used herein, “—S(O)R₅” refers to an sulfinyl group wherein R₅ is selected from hydrogen, alkyl, alkenyl, alkenyl, cycloalkyl, cycloalkenyl, cycloalkynyl, aryl, heteroaryl, heterocyclyl, halogen, sulfinyl, ester, carbonyl. As used herein, “ester” refers —C(O)OR₈ in which R₈ is selected from hydrogen, alkyl, alkenyl, alkenyl, cycloalkyl, cycloalkenyl, cycloalkynyl, aryl, heteroaryl, heterocyclyl, halogen, —NR₁R₂, sulfinyl, carbonyl, —OR₃, SR₄, —S(O)R₅—OH, —SH and —NH.

The term “substituted”, a ligand as defined herein above having (further substituted) one or more substituent, wherein the substituent is a ligand as defined herein above. In some embodiments the substituent is selected as specifically indicated and/or from a substituent selected from alkyl, alkenyl, alkynyl, cycloalkyl, cycloalkenyl, cycloalkynyl, aryl, heteroaryl, heterocyclyl, halogen, —NR₁R₂, —OR₃, —SR₄, —S(O)R₅, alkylene-COOH, ester, —OH, —SH, and —NH. In some embodiments, the number of substituent (on certain ligand) is 1 or 2 or 3 or 4 or 5 or 6 or 7 or 8 or 9 or 20 substituents.

In some embodiments, n is 1.

In some embodiments, n is 2.

In some embodiments, n is 3.

In some embodiments, n is 4.

In some embodiments, n is 5.

In some embodiments, in a compound of formula (I), ligand L is selected amongst ligands herein designated L1 through L63.

In some embodiments, in a compound according to formula (I), L is L1 or L2 or L3 or L4 or L5 or L6 or L7 or L8 or L9 or L10 or L11 or L12 or L13 or L14 or L15 or L16 or L17 or L18 or L19 or L20 or L21 or L22 or L23 or L24 or L25 or L26 or L27 or L28 or L29 or L30 or L31 or L32 or L33 or L34 or L35 or L36 or L37 or L38 or L39 or L40 or L41 or L42 or L43 or L44 or L45 or L46 or L47 or L48 or L49 or L50 or L51 or L52 or L53 or L54 or L55 or L56 or L57 or L58 or L59 or L60 or L61 or L62 or L63.

In some embodiments, L is selected from L1 or L2 or L3 or L4 or L5 or L6 or L7 or L8 or L9 or L10 or L11 or L12. In some embodiments, L is selected from L1, L2, L3, L4, L5, L6, L7, L8, L9, L10, L11, L12 L13, L14, L15, L16, L17, L18, L19, L20, L21, L22, L23, L24, L25, L26, L27, L28, L29, L30, L31, L32, L33, L34, L35, L36, L37, L38, L39, L40, L41, L42, L43, L44, L45, L46, L47, L48, L49, L50, L51, L52, L53, L54, L55, L56, L57, L58, L59, L60, L61, L62 and L63.

In some embodiments, in a compound of formula (I), wherein n is larger than 1, each L is independently selected from L1 through L63.

In some embodiments, in a compound of formula (I), wherein n is larger than 1, two ligands are identical and the rest of the ligands are independently L1 or L2 or L3 or L4 or L5 or L6 or L7 or L8 or L9 or L10 or L11 or L12 or L13 or L14 or L15 or L16 or L17 or L18 or L19 or L20 or L21 or L22 or L23 or L24 or L25 or L26 or L27 or L28 or L29 or L30 or L31 or L32 or L33 or L34 or L35 or L36 or L37 or L38 or L39 or L40 or L41 or L42 or L43 or L44 or L45 or L46 or L47 or L48 or L49 or L50 or L51 or L52 or L53 or L54 or L55 or L56 or L57 or L58 or L59 or L60 or L61 or L62 or L63.

In some embodiments, in a compound of formula (I), wherein n is larger than 1, two ligands are independently selected from L48 through L63, and the rest of the ligands are selected independently from L1 through L63.

In some embodiments, in a compound of formula (I), wherein n is larger than 1, two ligands are independently selected from L48 through L63, wherein said ligands are positioned in the axial positions of an octahedral complex, and the rest of the ligands are selected independently from L1 through L63.

In some embodiments, in a compound of formula (I), wherein n is larger than 1, two ligands are identical and selected from L48 through L63, and the rest of the ligands are selected independently from L1 through L63.

In some embodiments, in a compound of formula (I), wherein n is larger than 1, two ligands are identical and selected from L48 through L63, wherein said ligands are positioned in the axial positions of an octahedral complex, and the rest of the ligands are selected independently from L1 through L63.

In some embodiments, in a compound of formula (I), wherein n is larger than 1, one pairs of ligands are identical and selected from L48 through L63, and the rest of the ligands are selected independently from L1 through L63.

In some embodiments, in a compound of formula (I), wherein n is larger than 2, two pairs of ligands are identical and the rest of the ligands are L1 or L2 or L3 or L4 or L5 or L6 or L7 or L8 or L9 or L10 or L11 or L12 or L13 or L14 or L15 or L16 or L17 or L18 or L19 or L20 or L21 or L22 or L23 or L24 or L25 or L26 or L27 or L28 or L29 or L30 or L31 or L32 or L33 or L34 or L35 or L36 or L37 or L38 or L39 or L40 or L41 or L42 or L43 or L44 or L45 or L46 or L47 or L48 or L49 or L50 or L51 or L52 or L53 or L54 or L55 or L56 or L57 or L58 or L59 or L60 or L61 or L62 or L63.

In some embodiments, in a compound of formula (I), wherein n is larger than 2, two pairs of ligands are identical, one pair of ligands is selected from L1 through L47, the other pair is selected from L48 through L63, and the rest of the ligands are selected independently from L1 through L63.

In some embodiments, the compound of the invention comprises one or more phenylbutyrate ligand. In some embodiments, the compound of the invention is a compound of Formula (Ia):

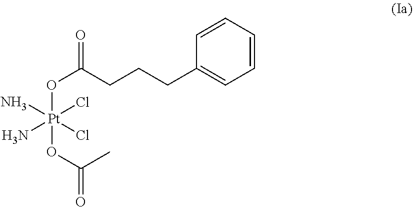

In some embodiments, the compound of the invention is a compound of Formula (II):

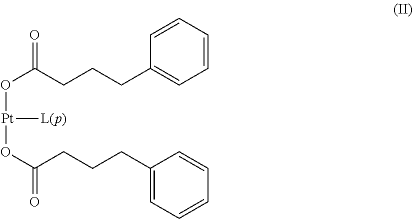

wherein L is as defined hereinabove; and p is the number of ligand moieties, being 1, 2, 3, or 4.

In some embodiments, p is 1.

In some embodiments, p is 2.

In some embodiments, p is 3.

In some embodiments, p is 4.

In some embodiments, the compound of the invention is a compound of Formula (IIa):

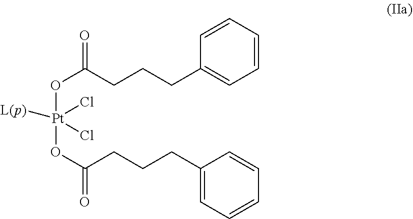

wherein L and p are as defined above.

In some embodiments, the compound of the invention is a compound of Formula (IIb):

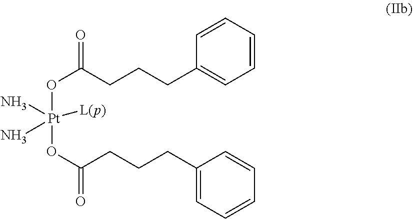

wherein L and p are as defined above.

In some embodiments, the compound of the invention is a compound of Formula (IIc):

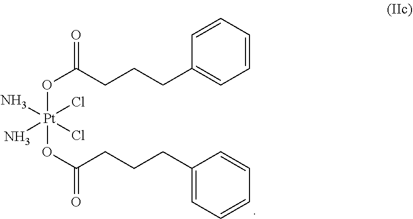

In some embodiments, the compound of the invention is a compound of Formula (IId):

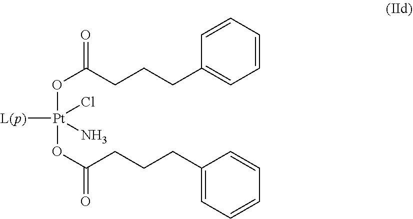

wherein L and p are as defined above.

In some embodiments, the compound of the invention is a compound of Formula (IIe):

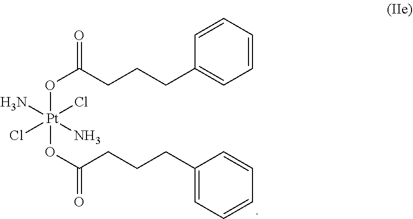

In some embodiments, the compound of the invention is a compound of Formula (IIf):

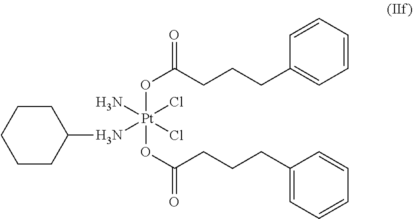

In some embodiments, the compound of the invention is a compound of Formula (IIg):

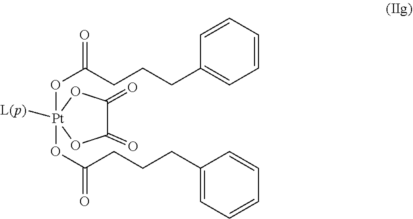

wherein L and p are as defined above.

In some embodiments, the compound of the invention is a compound of Formula (IIh):

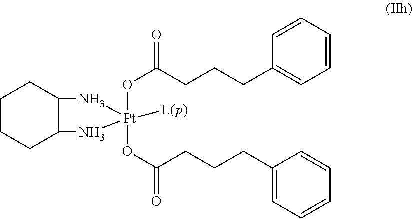

wherein L and p are as defined above.

In some embodiments, the compound of the invention is a compound of Formula

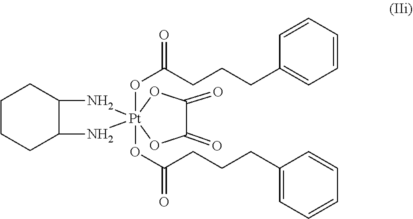

In some embodiments, the compound of the invention is a compound of Formula (IIj):

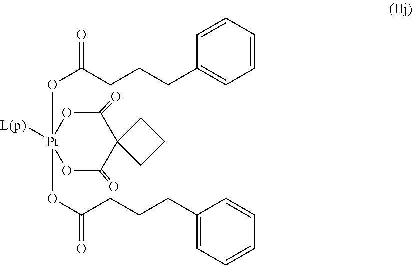

wherein L and p are as defined above.

In some embodiments, the compound of the invention is a compound of Formula (IIk):

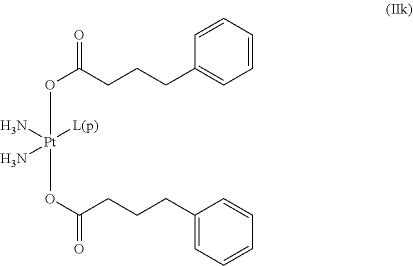

wherein L and p are as defined above.

In some embodiments, the compound of the invention is a compound of Formula (IIl):

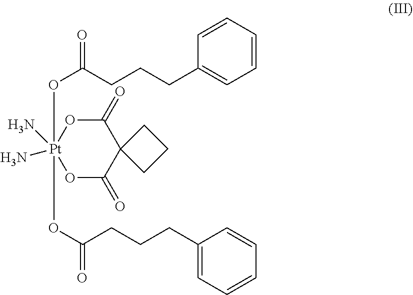

In some embodiments, the compound of the invention is a compound of Formula (IIm):

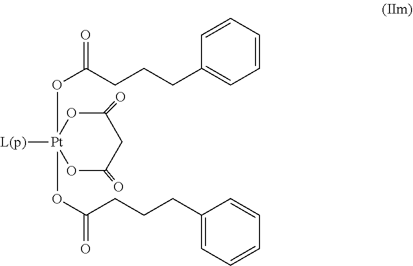

wherein L and p are as defined above.

In some embodiments, the compound of the invention is a compound of Formula (IIn):

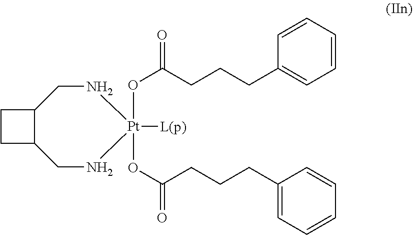

wherein L and p are as defined above.

In some embodiments, the compound of the invention is a compound of Formula (IIo):

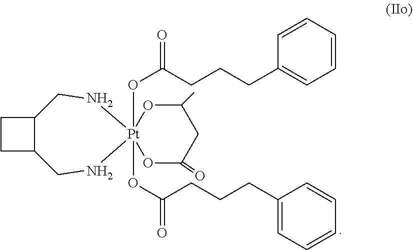

In some embodiments, the compound of the invention is a compound of Formula (IIp):

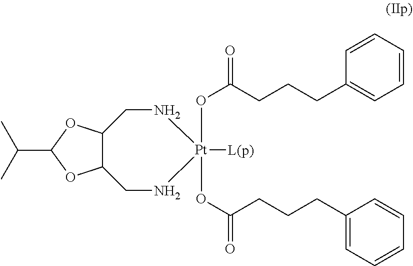

wherein L and p are as defined above.

In some embodiments, the compound of the invention is a compound of Formula (IIq):

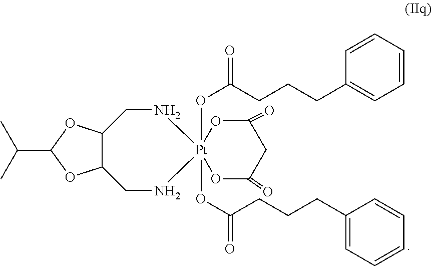

In some embodiments, the compound of the invention is a compound of Formula (IIr):

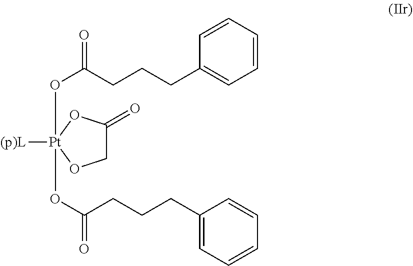

wherein L and p are as defined above.

In some embodiments, the compound of the invention is a compound of Formula (IIs):

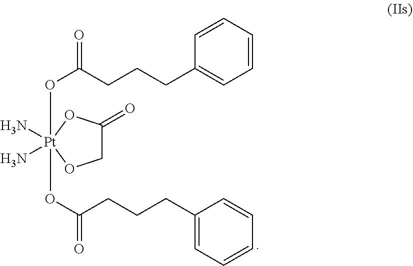

In some embodiments, the compound of the invention comprises one or more phenylbutyrate ligand and one or more therapeutically active anticancer moiety, as defined herein. In some embodiments, the one or more therapeutically active anticancer moiety is selected from pyruvate dehydrogenase kinase (PDK) inhibitors, histone deacetylase (HDAC) inhibitors, DNA methylation enhancers, COX inhibitors, PARP inhibitors, inhibitors of DNA repair and cancer targeting moieties.

In some embodiments, the compound of the invention is a compound of Formula (I):

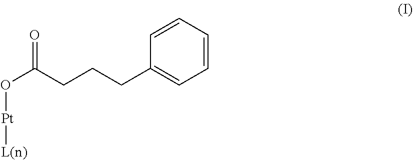

wherein L is a therapeutically active anticancer moiety and n is an integer as defined hereinabove. In some embodiments, L is selected from pyruvate dehydrogenase kinase (PDK) inhibitors, histone deacetylase (HDAC) inhibitors, DNA methylation enhancers, COX inhibitors, PARP inhibitors, inhibitors of DNA repair and cancer targeting moieties.

In some embodiments, the compound of the invention comprises two or more Pt atoms (centers), each Pt atom being linked to the other via a ligand group which may be selected amongst therapeutically active anticancer moieties, as defined, or via a linker moiety or group.

In some embodiments, the compound is of the Formula (III):

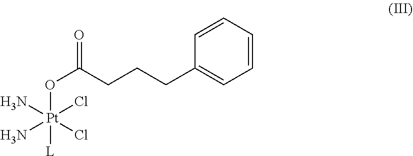

wherein L is a ligand different than phenylbutyrate; optionally being selected from therapeutically active anticancer moieties, as defined hereinabove.

In some embodiments, L is selected from pyruvate dehydrogenase kinase (PDK) inhibitors, histone deacetylase (HDAC) inhibitors, DNA methylation enhancers, COX inhibitors, PARP inhibitors, inhibitors of DNA repair and cancer targeting moieties.

In some embodiments, in a compound of Formula (III), L is selected from ligands herein designated L48 through L63. In some embodiments, in a compound of Formula (III), L is L48 or L49 or L50 or L51 or L52 or L53 or L54 or L55 or L56 or L57 or L58 or L59 or L60 or L61 or L62 or L63.

In some embodiments, the compound is a compound selected from:

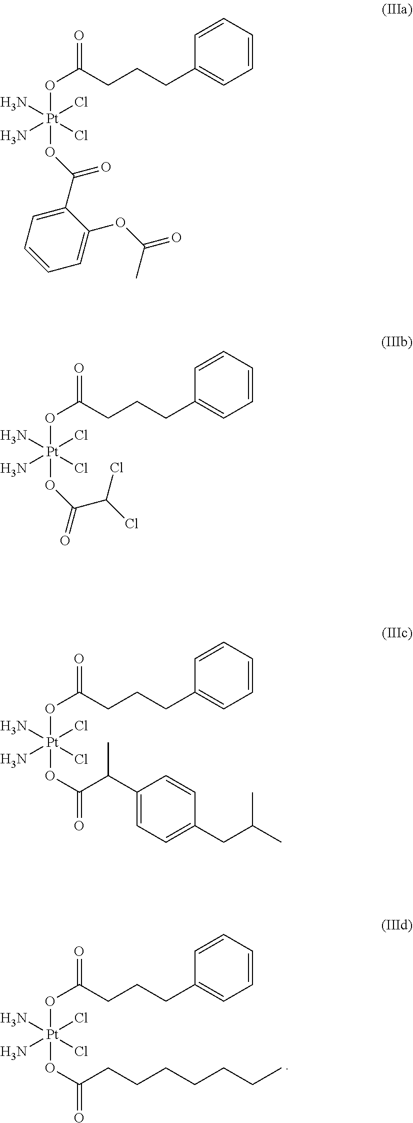

In some embodiments, the compound is of the Formula (IV):

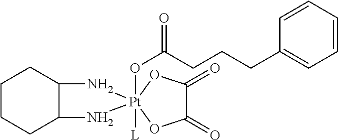

wherein L is a ligand different than phenylbutyrate; optionally being selected from therapeutically active anticancer moieties, as defined hereinabove.

In some embodiments, L is selected from pyruvate dehydrogenase kinase (PDK) inhibitors, histone deacetylase (HDAC) inhibitors, DNA methylation enhancers, COX inhibitors, PARP inhibitors, DNA repair inhibitors and cancer targeting moieties.

In some embodiments, in a compound of Formula (IV), L is selected from ligands herein designated L48 through L63. In some embodiments, in a compound of Formula (IV), L is L48 or L49 or L50 or L51 or L52 or L53 or L54 or L55 or L56 or L57 or L58 or L59 or L60 or L61 or L62 or L63.

In some embodiments, the compound is a compound selected from:

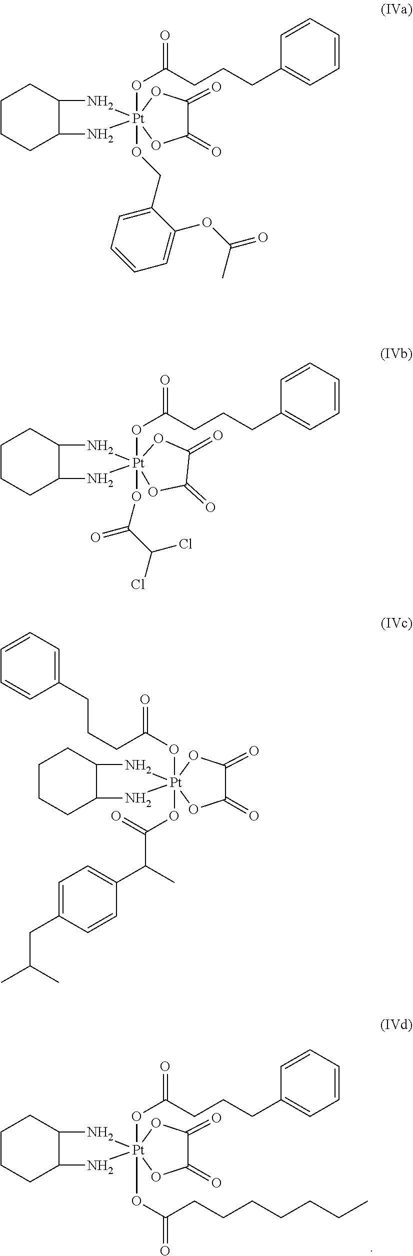

In some embodiments, the compound is of the Formula (V):

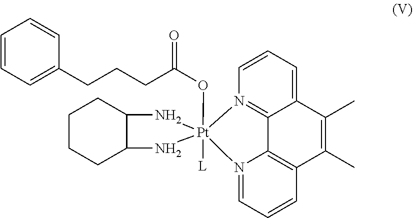

wherein L is a ligand different than phenylbutyrate; optionally being selected from therapeutically active anticancer moieties, as defined hereinabove.

In some embodiments, L is selected from pyruvate dehydrogenase kinase (PDK) inhibitors, histone deacetylase (HDAC) inhibitors, DNA methylation enhancers, COX inhibitors, PARP inhibitors, DNA repair inhibitors and cancer targeting moieties.

In some embodiments, in a compound of Formula (V), L is selected from ligands herein designated L48 through L63. In some embodiments, in a compound of Formula (V), L is L48 or L49 or L50 or L51 or L52 or L53 or L54 or L55 or L56 or L57 or L58 or L59 or L60 or L61 or L62 or L63.

In some embodiments, the compound is a compound selected from:

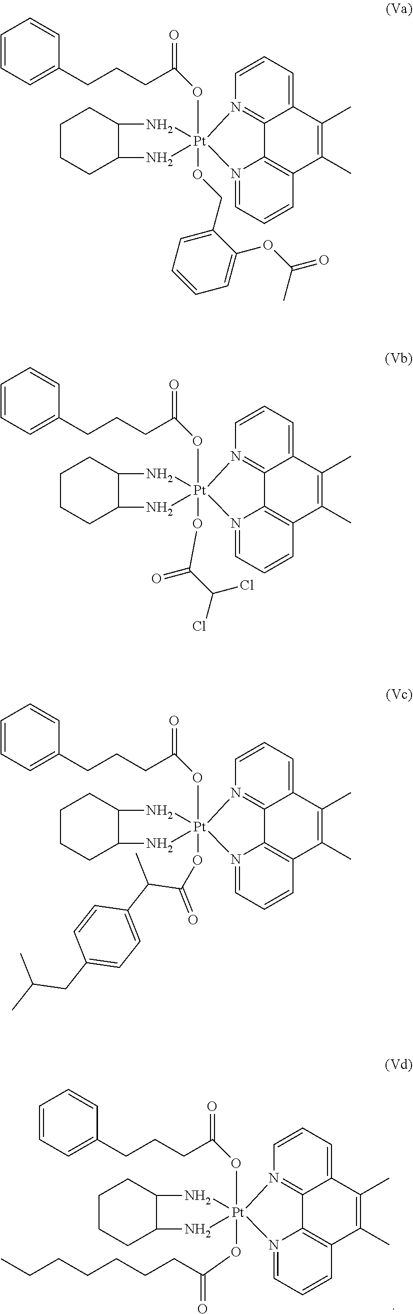

In some embodiments, the compound is of the Formula (VI):

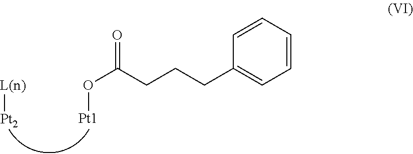

wherein L is a ligand selected as defined herein; n is the number of ligands, selected as herein. The compound of Formula (VI) comprises two or more Pt atoms, each may be labeled Pt1, Pt2, Pt3 . . . , and may be independently substituted with or associated to the same or different ligands or groups or therapeutically active anticancer moieties. In such compounds of the invention, each Pt atom is connected to another Pt atom via a linker group or a ligand designated in Formula (VI) by a curved line, and each Pt atom being connected to one or more ligand groups as disclosed herein. As shown in the compound of Formula (VI), in some embodiments, at least one ligand is phenylbutyrate.

In some embodiments, each of the Pt atoms is bonded to at least one ligand herein designated L1 through L63.

In some embodiments, a Pt atom in a compound of Formula (VI) may be associated to at least one therapeutically active anticancer moieties, as defined hereinabove, optionally selected from pyruvate dehydrogenase kinase (PDK) inhibitors, histone deacetylase (HDAC) inhibitors, DNA methylation enhancers, COX inhibitors, PARP inhibitors, DNA repair inhibitors and cancer targeting moieties.

In some embodiments, in a compound of Formula (VI), one or more of the Pt atoms is associated to a ligand herein designated L48 through L63.

In some embodiments, the linker group linking the two or more Pt atoms may be any carbon chain comprising between 6 and 25 carbon atoms.

In some embodiments, the linker group linking the two or more Pt atoms may be a ligand group selected from:
L49, wherein each of n and m independently is an integer between 0 and 6; the ligand associates to the Pt via the oxygen atom(s);

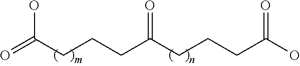

L50, wherein each of n and m independently is an integer between 0 and 6; the ligand associates to the Pt via the oxygen atom(s) and/or the nitrogen atom(s);

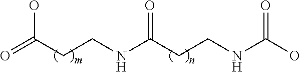

L51, wherein each of n and m independently is an integer between 0 and 6; the ligand associates to the Pt via the oxygen atom(s) and/or the nitrogen atom;

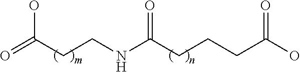

and
L52, wherein each of n and m independently is an integer between 0 and 6; the ligand associates to the Pt via the oxygen atom(s) and/or the nitrogen atom(s)

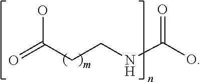

In some embodiments, the linker group is a ligand selected from: L49, wherein each of n and m is between 1 and 3; L50, wherein each of n and m is between 1 and 3; L51, wherein each of n and m is between 0 and 3; and L52, wherein each of n and m is between 1 and 3.

In some embodiments, the linker group is:

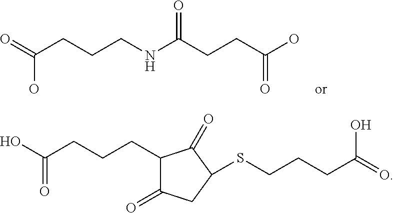 [SMILES: O=C(O)CCCNC(=O)CCC(=O)O ; O=C(O)CCCSC1CC(=O)C(CCCC(=O)O)C1=O]

In some embodiments, the compound is a compound of the Formula (VII):

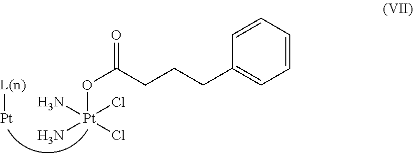

wherein L and n are each as defined herein.

In some embodiments, the linker group linking the two or more Pt atoms may be any carbon chain comprising between 6 and 25 carbon atoms.

In some embodiments, the linker group linking the two or more Pt atoms may be a ligand group selected from:
L49, wherein each of n and m independently is an integer between 0 and 6; the ligand associates to the Pt via the oxygen atom(s);

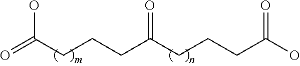

L50, wherein each of n and m independently is an integer between 0 and 6; the ligand associates to the Pt via the oxygen atom(s) and/or the nitrogen atom(s);

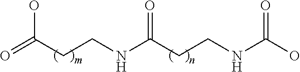

L51, wherein each of n and m independently is an integer between 0 and 6; the ligand associates to the Pt via the oxygen atom(s) and/or the nitrogen atom;

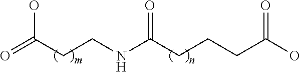

and
L52, wherein each of n and m independently is an integer between 0 and 6; the ligand associates to the Pt via the oxygen atom(s) and/or the nitrogen atom(s);

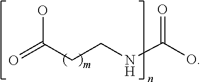

In some embodiments, the linker group is a ligand selected from: L49, wherein each of n and m is between 1 and 3; L50, wherein each of n and m is between 1 and 3; L51, wherein each of n and m is between 0 and 3; and L52, wherein each of n and m is between 1 and 3.

In some embodiments, the linker group is:

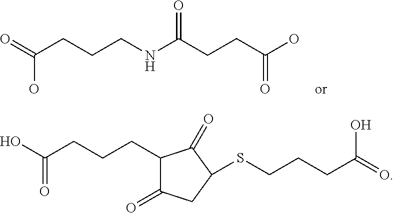 [SMILES: O=C(O)CCCNC(=O)CCC(=O)O ; O=C(O)CCCSC1CC(=O)C(CCCC(=O)O)C1=O]

In some embodiments, the compound is a compound of the Formula (VIII):

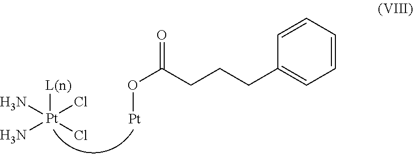

wherein L and n are each as defined herein.

In some embodiments, the linker group linking the two or more Pt atoms may be any carbon chain comprising between 6 and 25 carbon atoms.

In some embodiments, the linker group linking the two or more Pt atoms may be a ligand group selected from:
L49, wherein each of n and m independently is an integer between 0 and 6; the ligand associates to the Pt via the oxygen atom(s);

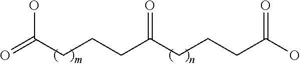

L50, wherein each of n and m independently is an integer between 0 and 6; the ligand associates to the Pt via the oxygen atom(s) and/or the nitrogen atom(s);

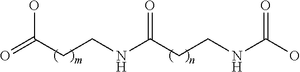

L51, wherein each of n and m independently is an integer between 0 and 6; the ligand associates to the Pt via the oxygen atom(s) and/or the nitrogen atom;

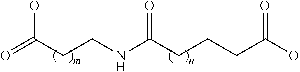

and
L52, wherein each of n and m independently is an integer between 0 and 6; the ligand associates to the Pt via the oxygen atom(s) and/or the nitrogen atom(s);

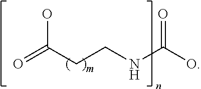

In some embodiments, the linker group is a ligand selected from: L49, wherein each of n and m is between 1 and 3; L50, wherein each of n and m is between 1 and 3; L51, wherein each of n and m is between 0 and 3; and L52, wherein each of n and m is between 1 and 3.

In some embodiments, the linker group is:

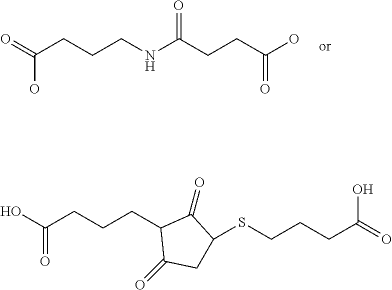 [SMILES: O=C(O)CCCNC(=O)CCC(=O)O ; O=C(O)CCCSC1CC(=O)C(CCCC(=O)O)C1=O]

In some embodiments, the compound is a compound of the Formula (IX):

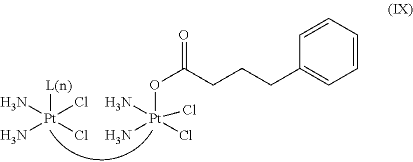

wherein L and n are each as defined herein.

In some embodiments, the linker group linking the two or more Pt atoms may be any carbon chain comprising between 6 and 25 carbon atoms.

In some embodiments, the linker group linking the two or more Pt atoms may be a ligand group selected from:
L49, wherein each of n and m independently is an integer between 0 and 6; the ligand associates to the Pt via the oxygen atom(s);

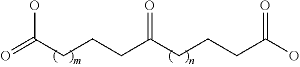

L50, wherein each of n and m independently is an integer between 0 and 6; the ligand associates to the Pt via the oxygen atom(s) and/or the nitrogen atom(s);

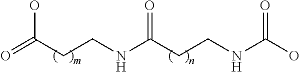

L51, wherein each of n and m independently is an integer between 0 and 6; the ligand associates to the Pt via the oxygen atom(s) and/or the nitrogen atom;

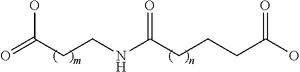

and
L52, wherein each of n and m independently is an integer between 0 and 6; the ligand associates to the Pt via the oxygen atom(s) and/or the nitrogen atom(s);

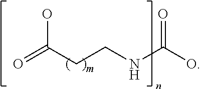

In some embodiments, the linker group is a ligand selected from: L49, wherein each of n and m is between 1 and 3; L50, wherein each of n and m is between 1 and 3; L51, wherein each of n and m is between 0 and 3; and L52, wherein each of n and m is between 1 and 3.

In some embodiments, the linker group is:

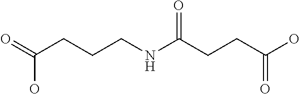 [SMILES: O=C(O)CCCNC(=O)CCC(=O)O]

In some embodiments, compounds of Formulae (VI) through (IX), are selected from Formulae (X), (XI) and (XII):

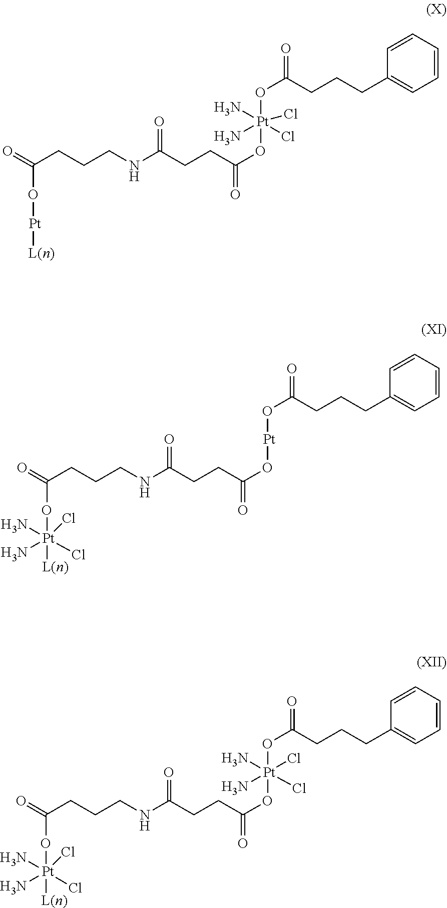

wherein L and n are each as defined herein.

In some embodiments, in a compound of any one of Formulae (X), (XI) and (XII), each of L may be selected from L48 through L63.

In some embodiments, compounds of the invention are of Formulae (XIII) through (XV):

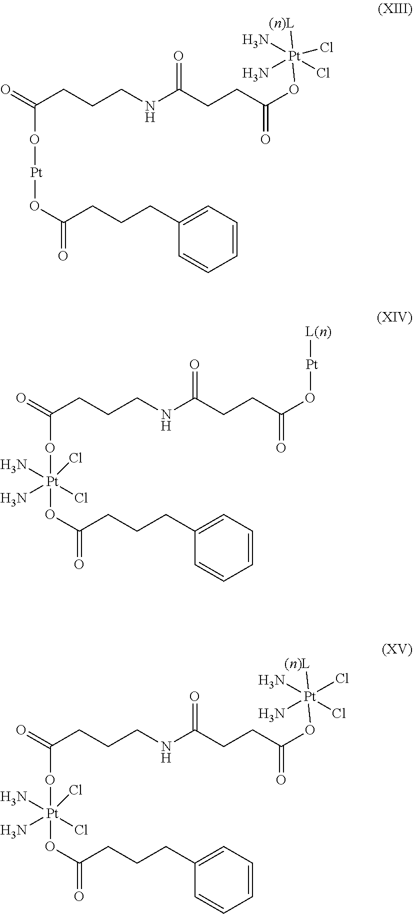

wherein each of L and n are as defined herein.

In some embodiments, the compound is a compound of the Formula (XVI):

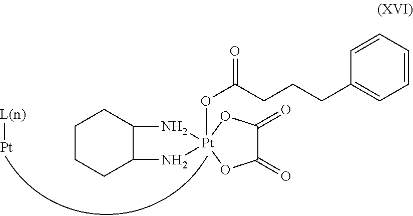

wherein L and n are each as defined herein.

In some embodiments, the linker group linking the two or more Pt atoms may be any carbon chain comprising between 6 and 25 carbon atoms.

In some embodiments, the linker group linking the two or more Pt atoms may be a ligand group selected from:
L49, wherein each of n and m independently is an integer between 0 and 6; the ligand associates to the Pt via the oxygen atom(s);

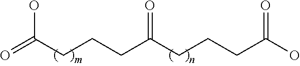

L50, wherein each of n and m independently is an integer between 0 and 6; the ligand associates to the Pt via the oxygen atom(s) and/or the nitrogen atom(s);

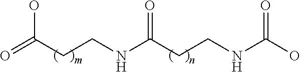

L51, wherein each of n and m independently is an integer between 0 and 6; the ligand associates to the Pt via the oxygen atom(s) and/or the nitrogen atom;

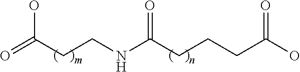

and
L52, wherein each of n and m independently is an integer between 0 and 6; the ligand associates to the Pt via the oxygen atom(s) and/or the nitrogen atom(s);

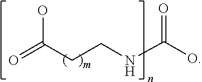

In some embodiments, the linker group is a ligand selected from: L49, wherein each of n and m is between 1 and 3; L50, wherein each of n and m is between 1 and 3; L51, wherein each of n and m is between 0 and 3; and L52, wherein each of n and m is between 1 and 3.

In some embodiments, the linker group is:

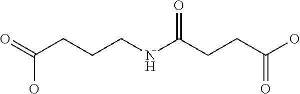 [SMILES: O=C(O)CCCNC(=O)CCC(=O)O]

In some embodiments, the compound is a compound of the Formula (XVII):

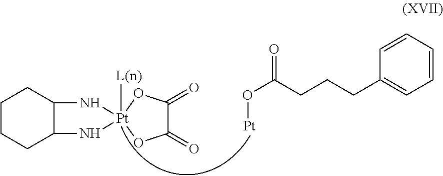

wherein L and n are each as defined herein.

In some embodiments, the linker group linking the two or more Pt atoms may be a ligand group selected from:
L49, wherein each of n and m independently is an integer between 0 and 6; the ligand associates to the Pt via the oxygen atom(s);

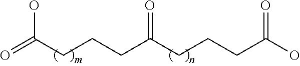

L50, wherein each of n and m independently is an integer between 0 and 6; the ligand associates to the Pt via the oxygen atom(s) and/or the nitrogen atom(s);

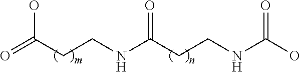

L51, wherein each of n and m independently is an integer between 0 and 6; the ligand associates to the Pt via the oxygen atom(s) and/or the nitrogen atom;

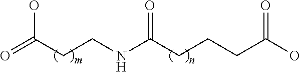

and
L52, wherein each of n and m independently is an integer between 0 and 6; the ligand associates to the Pt via the oxygen atom(s) and/or the nitrogen atom(s);

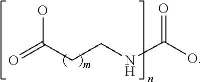

In some embodiments, the linker group is a ligand selected from: L49, wherein each of n and m is between 1 and 3; L50, wherein each of n and m is between 1 and 3; L51, wherein each of n and m is between 0 and 3; and L52, wherein each of n and m is between 1 and 3.

In some embodiments, the linker group is:

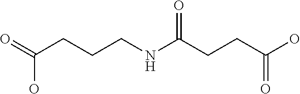 [SMILES: O=C(O)CCCNC(=O)CCC(=O)O]

In some embodiments, the compound is a compound of the Formula (XVIII):

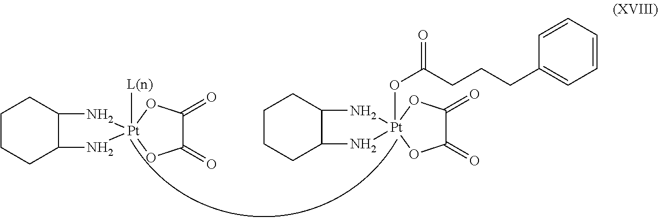

wherein L and n are each as defined herein.

In some embodiments, the linker group linking the two or more Pt atoms may be a ligand group selected from:
L49, wherein each of n and m independently is an integer between 0 and 6; the ligand associates to the Pt via the oxygen atom(s);

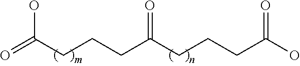

L50, wherein each of n and m independently is an integer between 0 and 6; the ligand associates to the Pt via the oxygen atom(s) and/or the nitrogen atom(s);

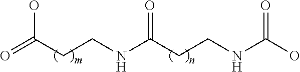

L51, wherein each of n and m independently is an integer between 0 and 6; the ligand associates to the Pt via the oxygen atom(s) and/or the nitrogen atom;

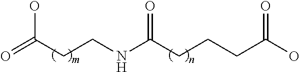

and
L52, wherein each of n and m independently is an integer between 0 and 6; the ligand associates to the Pt via the oxygen atom(s) and/or the nitrogen atom(s);

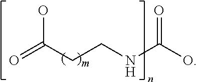

In some embodiments, the linker group is a ligand selected from: L49, wherein each of n and m is between 1 and 3; L50, wherein each of n and m is between 1 and 3; L51, wherein each of n and m is between 0 and 3; and L52, wherein each of n and m is between 1 and 3.

In some embodiments, the linker group is:

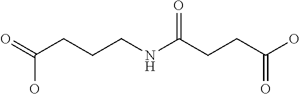 [SMILES: O=C(O)CCCNC(=O)CCC(=O)O]

In some embodiments, compounds of the invention are of Formulae (XIX), (XX) and (XXI):

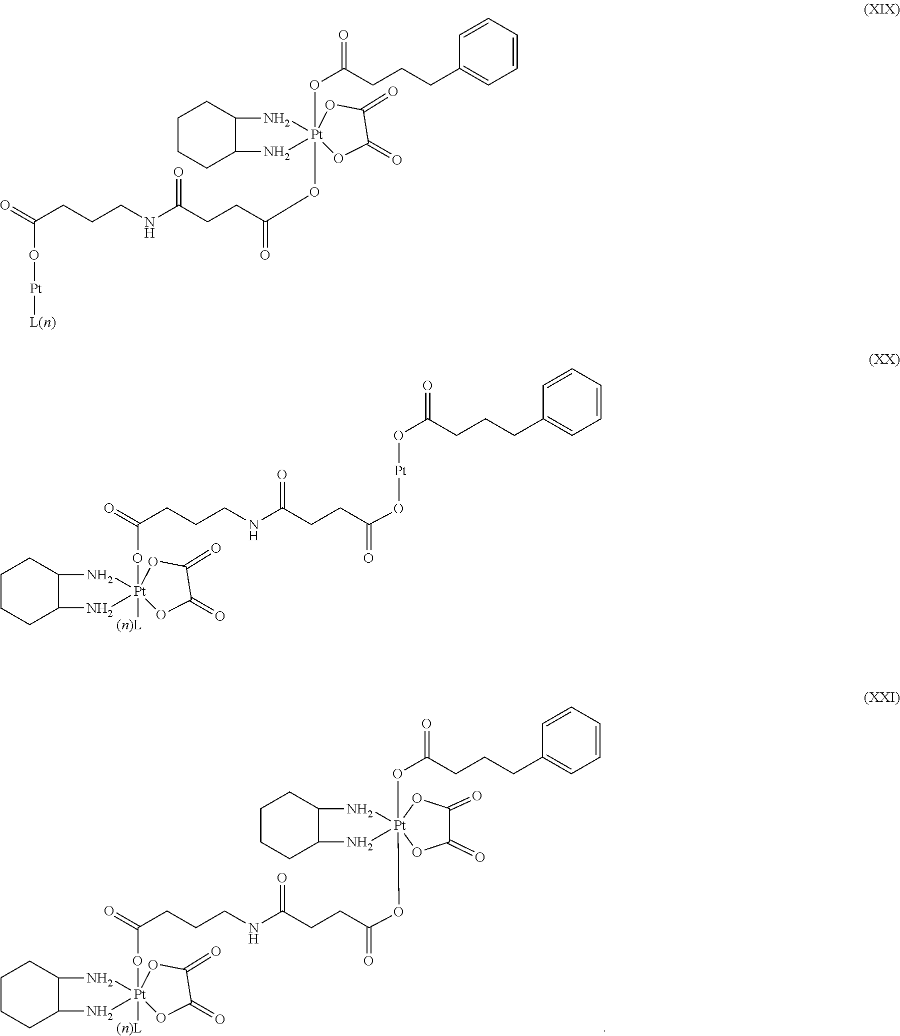

In some embodiments, in a compound of any one of Formulae (XIX), (XX) and (XXI), each of L may be selected from L48 through L63.

In some embodiments, compounds of the invention are of Formulae (XXII) through (XXIV):

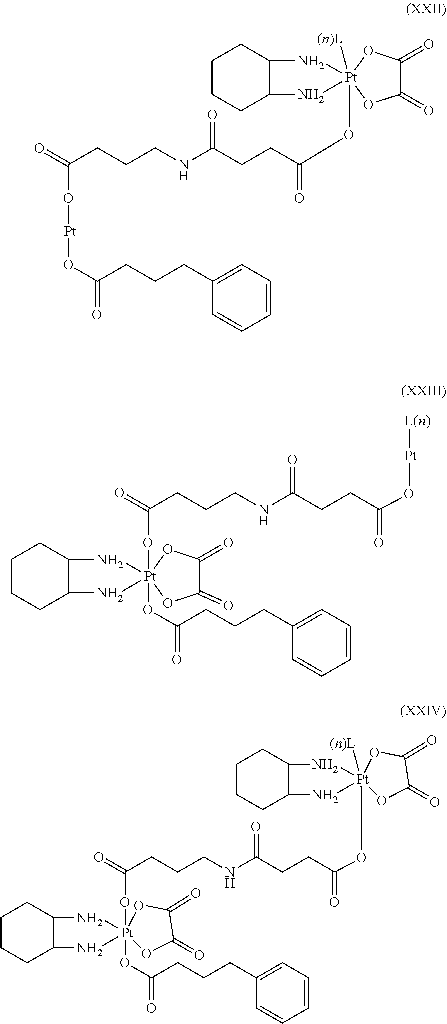

wherein each of L and n is as defined herein.

In some embodiments, compounds of the invention are of Formulae (XXV) and (XXVI):

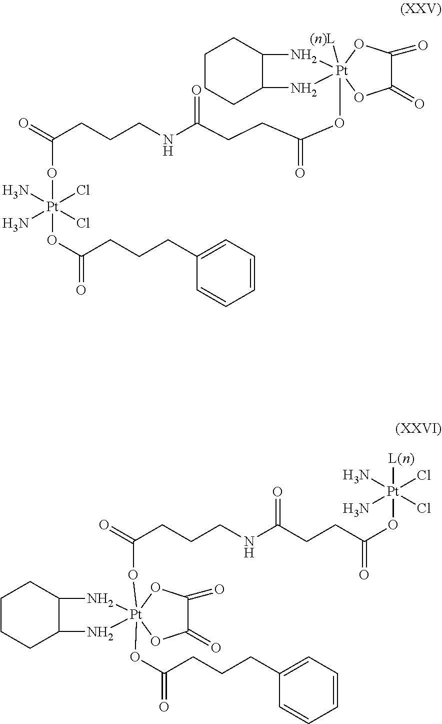

wherein each of L and n is as defined herein.

In some embodiments, compounds of the invention are of Formulae (XXVII) and (XXVIII):

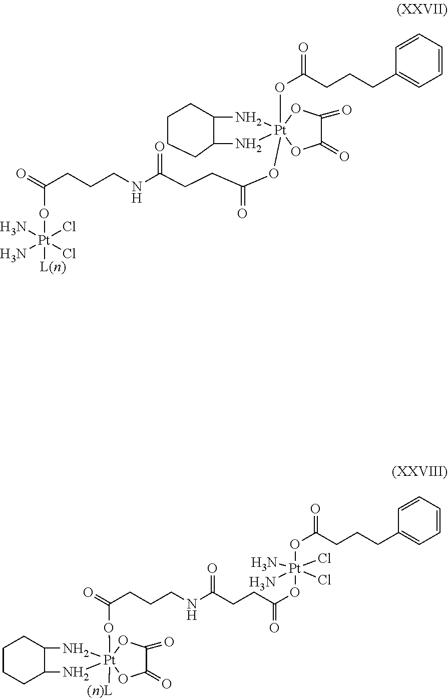

wherein each of L and n is as defined herein.

In some embodiments, compounds of the invention are of the Formula (XXIX):

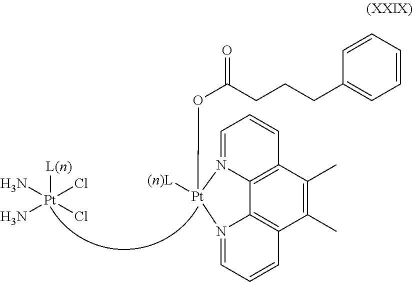

wherein L and n are each as defined herein.

In some embodiments, the linker group linking the two or more Pt atoms may be a ligand group selected from:
L49, wherein each of n and m independently is an integer between 0 and 6; the ligand associates to the Pt via the oxygen atom(s);

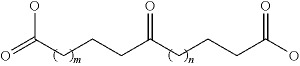

L50, wherein each of n and m independently is an integer between 0 and 6; the ligand associates to the Pt via the oxygen atom(s) and/or the nitrogen atom(s);

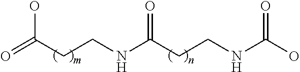

L51, wherein each of n and m independently is an integer between 0 and 6; the ligand associates to the Pt via the oxygen atom(s) and/or the nitrogen atom;

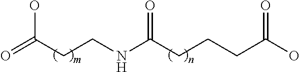

and
L52, wherein each of n and m independently is an integer between 0 and 6; the ligand associates to the Pt via the oxygen atom(s) and/or the nitrogen atom(s);

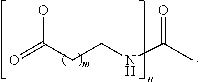

In some embodiments, the linker group is a ligand selected from: L49, wherein each of n and m is between 1 and 3; L50, wherein each of n and m is between 1 and 3; L51, wherein each of n and m is between 0 and 3; and L52, wherein each of n and m is between 1 and 3.

In some embodiments, the linker group is:

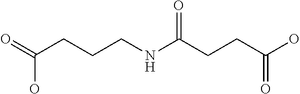 [SMILES: O=C(O)CCCNC(=O)CCC(=O)O]

In some embodiments, the compound is a compound of the Formula (XXX):

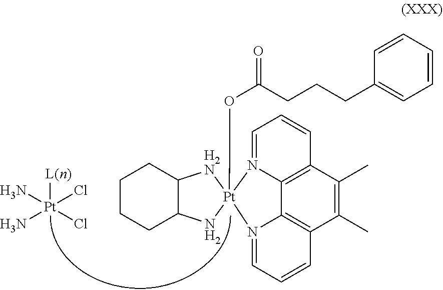

wherein L and n are each as defined herein.

In some embodiments, the linker group linking the two or more Pt atoms may be a ligand group selected from:
L49, wherein each of n and m independently is an integer between 0 and 6; the ligand associates to the Pt via the oxygen atom(s);

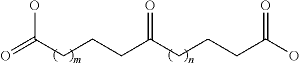

L50, wherein each of n and m independently is an integer between 0 and 6; the ligand associates to the Pt via the oxygen atom(s) and/or the nitrogen atom(s);

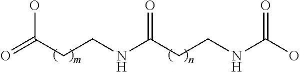

L51, wherein each of n and m independently is an integer between 0 and 6; the ligand associates to the Pt via the oxygen atom(s) and/or the nitrogen atom;

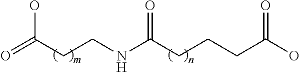

and
L52, wherein each of n and m independently is an integer between 0 and 6; the ligand associates to the Pt via the oxygen atom(s) and/or the nitrogen atom(s);

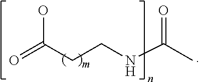

In some embodiments, the linker group is a ligand selected from: L49, wherein each of n and m is between 1 and 3; L50, wherein each of n and m is between 1 and 3; L51, wherein each of n and m is between 0 and 3; and L52, wherein each of n and m is between 1 and 3.

In some embodiments, the linker group is:

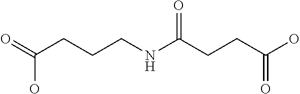 [SMILES: O=C(O)CCCNC(=O)CCC(=O)O]

In some embodiments, compounds of the invention are of Formulae (XXXI):

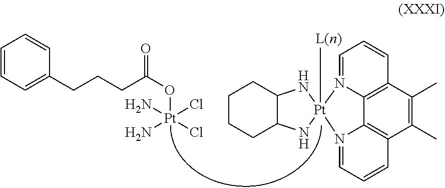

In some embodiments, in a compound of Formula (XXXI), L may be selected from L48 through L63.

In another aspect, the invention further provides a compound selected from Compounds herein designated:

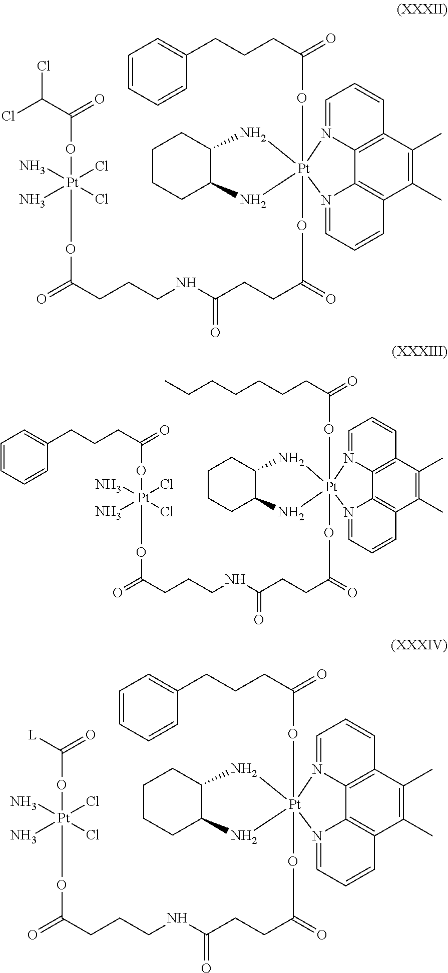

wherein L is a therapeutically active anticancer moiety as disclosed herein.

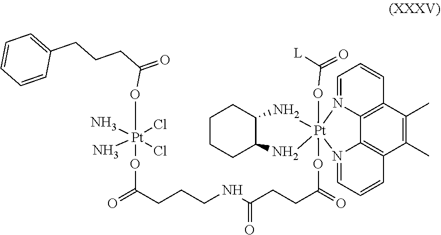

wherein L is a therapeutically active anticancer moiety as disclosed herein,

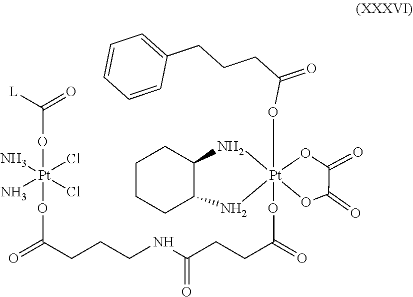

wherein L is a therapeutically active anticancer moiety as disclosed herein,

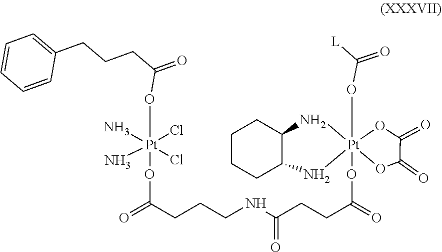

wherein L is a therapeutically active anticancer moiety as disclosed herein,

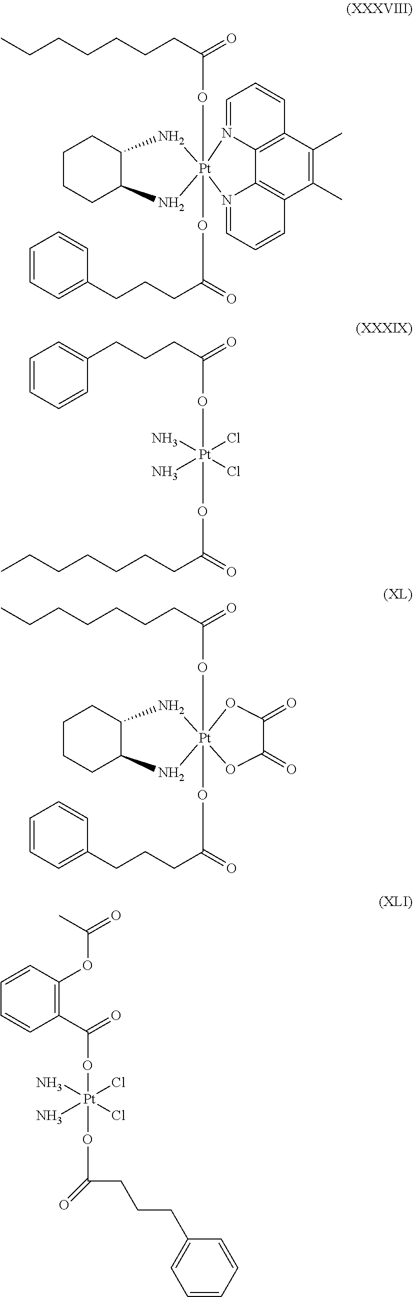

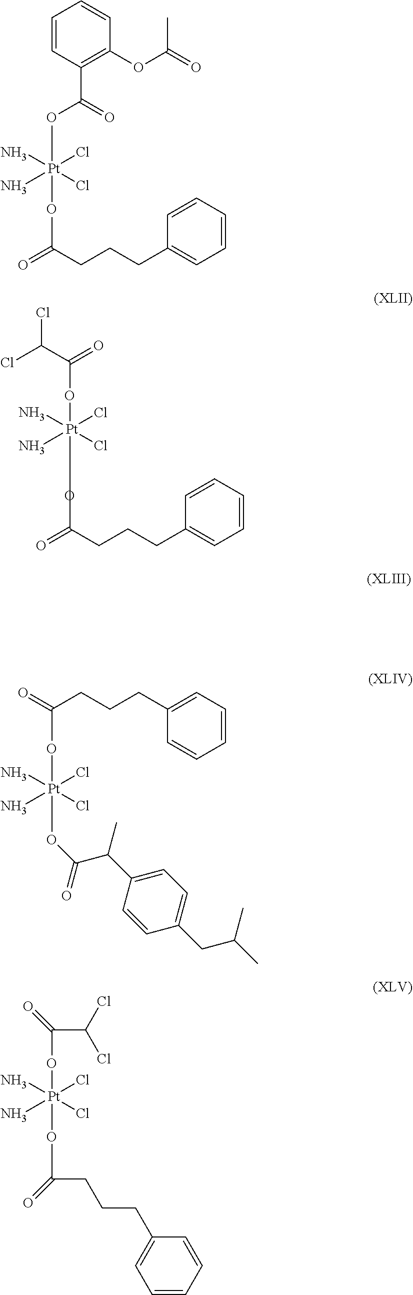

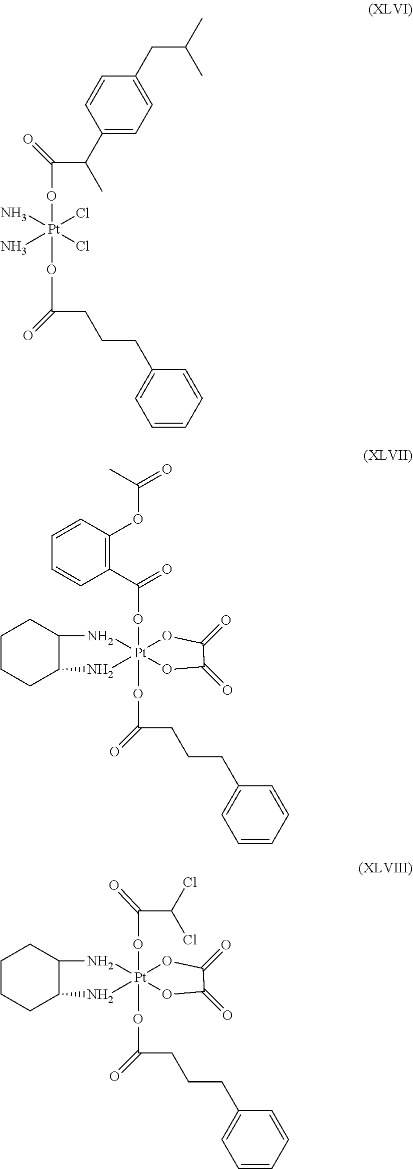

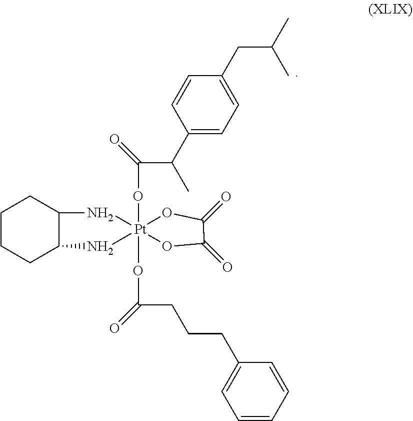

In another aspect, the invention provides a compound selected from:

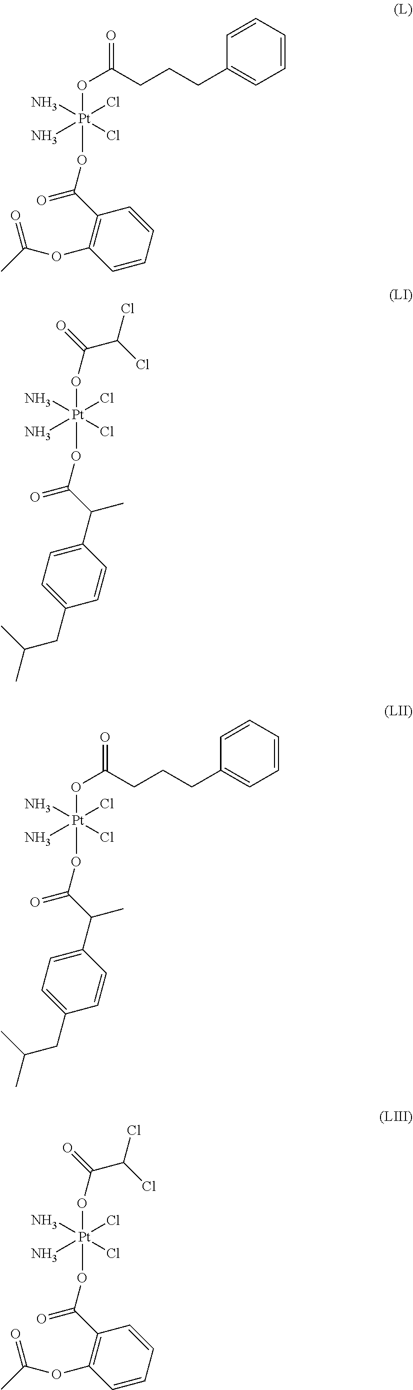

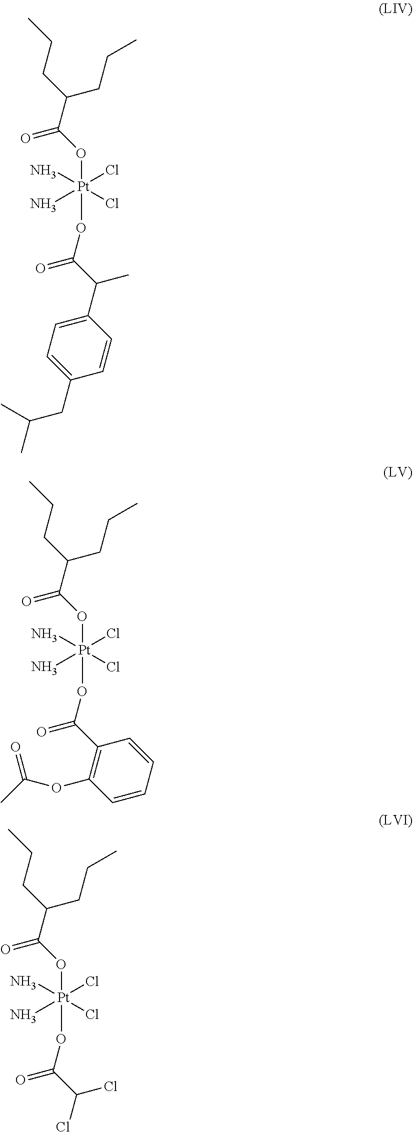

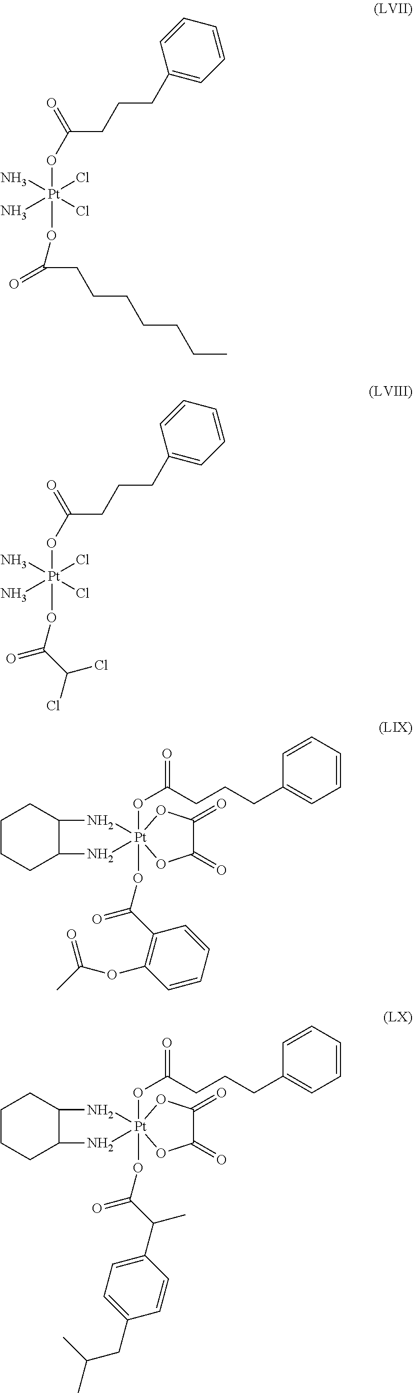

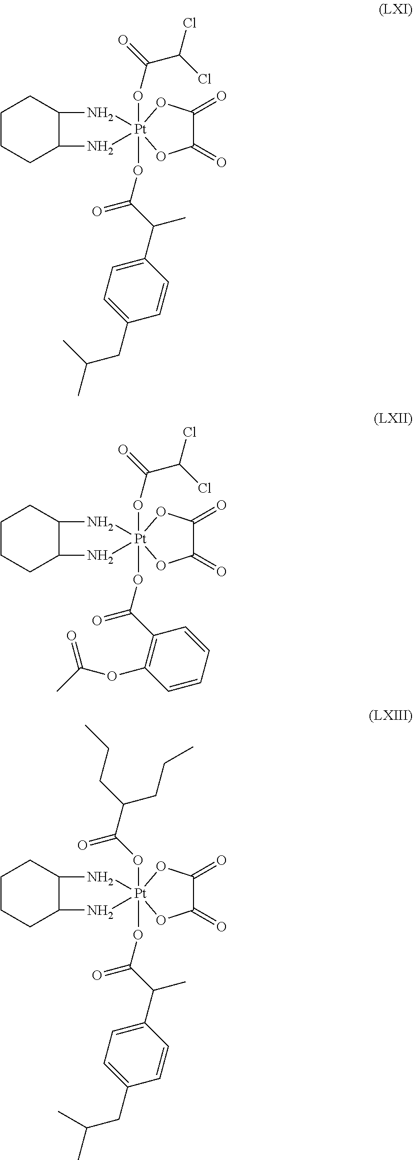

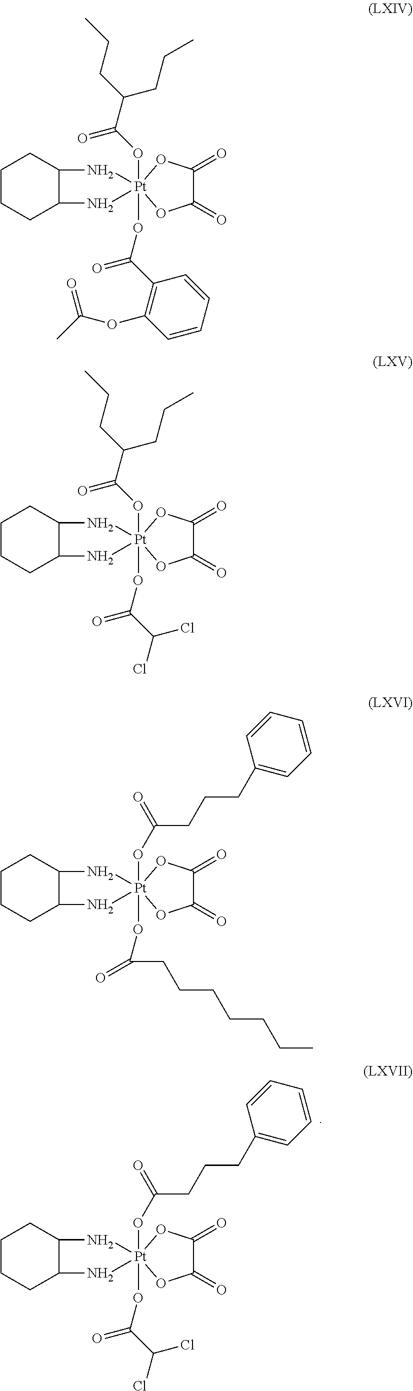

In some embodiments of compounds of the invention, the compounds are not of formula (M):

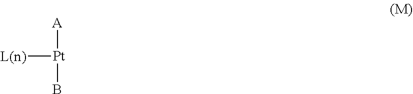

wherein

Pt is a platinum atom;

A is a C₈-C₂₂ fatty acid associated with the Pt atom via an oxygen atom of the fatty acid;

B is a C₂-C₂₂ fatty acid associated with the Pt atom via an oxygen atom of the fatty acid;

provided that each of A and B is not C₆-C₉ branched alkyl fatty acid;

L is a ligand atom or group of atoms selected from substituted or unsubstituted alkyl, substituted or unsubstituted alkenyl, substituted or unsubstituted alkynyl, substituted or unsubstituted cycloalkyl, substituted or unsubstituted cycloalkenyl, substituted or unsubstituted cycloalkynyl, substituted or unsubstituted aryl, substituted or unsubstituted heteroaryl, substituted or unsubstituted heterocyclyl, halide atom, substituted or unsubstituted amine —NR₁R₂, substituted or unsubstituted —OR₃, substituted or unsubstituted —SR₄, substituted or unsubstituted —S(O)R₅, substituted or unsubstituted alkylene-COOH, —OH, —SH, —NH, or any one of ligands L1 to L5 as designated herein; and

n is the number of ligand moieties, being 1, 2, 3, or 4;

R₁ and R₂ are each independently selected from hydrogen, alkyl, alkenyl, alkenyl, cycloalkyl, cycloalkenyl, cycloalkynyl, aryl, heteroaryl, heterocyclyl, halide, —C(O)NR₆R₇, sulfinyl, ester, and carbonyl; or wherein R₁ and R₂ in form a cyclic structure with the N atom they are bonded to;

each of R₃, R₄, and 123 is independently selected from hydrogen, alkyl, alkenyl, alkenyl, cycloalkyl, cycloalkenyl, cycloalkynyl, aryl, heteroaryl, heterocyclyl, halide, sulfinyl, ester, and carbonyl; and

R₆ and R₇ are each independently selected from hydrogen, alkyl, alkenyl, alkenyl, cycloalkyl, cycloalkenyl, cycloalkynyl, aryl, heteroaryl, heterocyclyl, halide, sulfinyl, ester, carbonyl, —OH, —SH and —NH.

In some embodiments, excluded is Oxaliplatin palmitate acetate.

In some embodiments, excluded are Compounds of formula (M):
wherein A is selected from octanoic (caprylic) acid, nonanoic (pelargonic) acid, decanoic (capric) acid, undecanoic (undecylic) acid, dodecanoic (lauric) acid, tridecanoic (tridecylic) acid, tetradecanoic (myristic) acid, pentadecanoic (pentadecylic) acid, hexadecanoic (palmitic) acid, heptadecanoic (margaric) acid, octadecanoic (stearic) acid, nonadecanoic (nonadecylic) acid, eicosanoic (arachidic) acid, heneicosanoic (heneicosylic) acid and docosanoic (Behenic) acid.
wherein B is a C₂-C₇ fatty acid.
wherein B is selected from propanoic (propionic) acid, butanoic (butyric) acid, pentanoic (valeric) acid, hexanoic (caproic) acid and heptanoic (enanthic) acid.
wherein the platinum atom is selected from platinum(III), platinum(IV), platinum(V) and platinum(VI)
wherein at least one ligand L is a halide selected from F, Cl, Br, and I.
wherein said halide is Cl.
wherein at least one ligand L is an amine
wherein at least one other ligand L is an amine
being a compound of formula (M1),

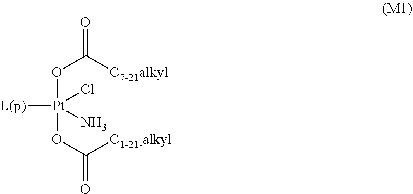

wherein L is as defined in claim 1 and p is the number of ligand moieties, being 0, 1 or 2.
wherein n is 2, 3 or 4, and wherein at least 2 ligands L are identical.
wherein L is a halide or an amine
being selected from compounds of formulae (M2) and (M3):

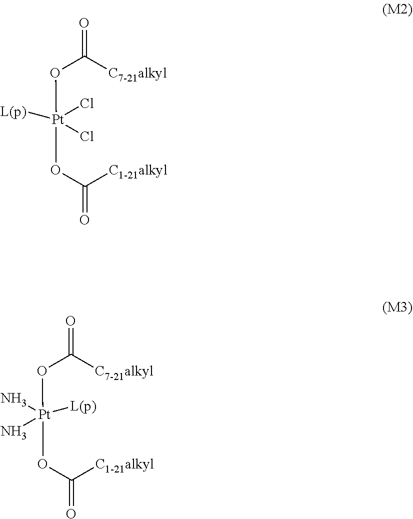

wherein L is independently as defined in claim 1 and p in Formula (M4) is 0, 1 or 2.
wherein at least 2 ligands L are halides and the other L ligands are amines
wherein at least 2 ligands L are amines and the other L ligands are halides.
being a compound of formula (M5) or (M6):

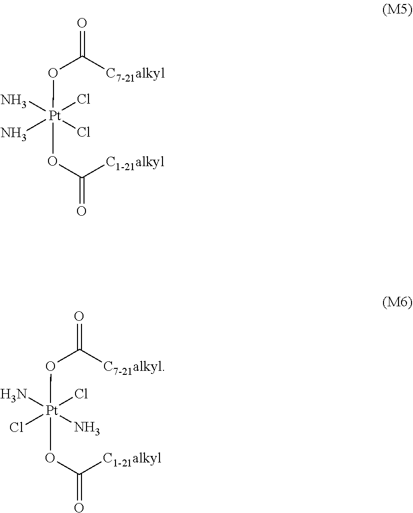

wherein at least one ligand L is L1 and the compound having the formula (M7):

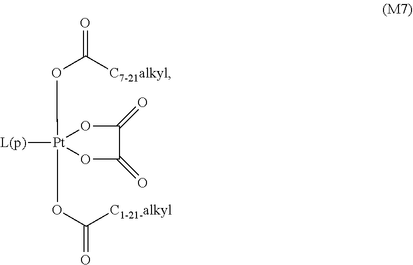

wherein L is as defined in claim 1 and p is the number of ligand moieties, being 0, 1 or 2.
wherein L is —NR₁R₂, wherein R₁ and R₂ are as defined in claim 1.
wherein at least one ligand L is L4 and the compound having the formula (M8):

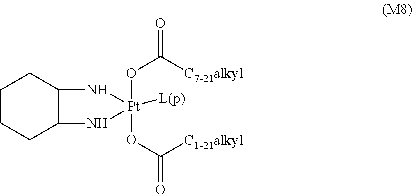

wherein L is as defined in claim 1 and p is the number of ligand moieties, being 0, 1 or 2.
wherein at least one other ligand L is a halide or an amine
being a compound of formula (M9):

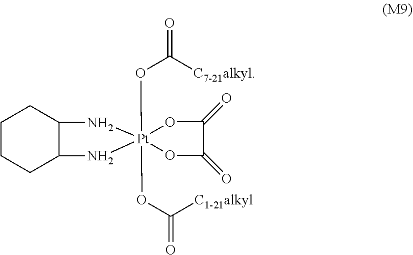

wherein at least one ligand L is bound to the platinum atom via at least one heteroatom selected from nitrogen, oxygen and sulfur.
wherein at least one of the bonds between the platinum atom and the heteroatoms are covalent and the other remaining bonds are coordinative bonds.
being selected from:

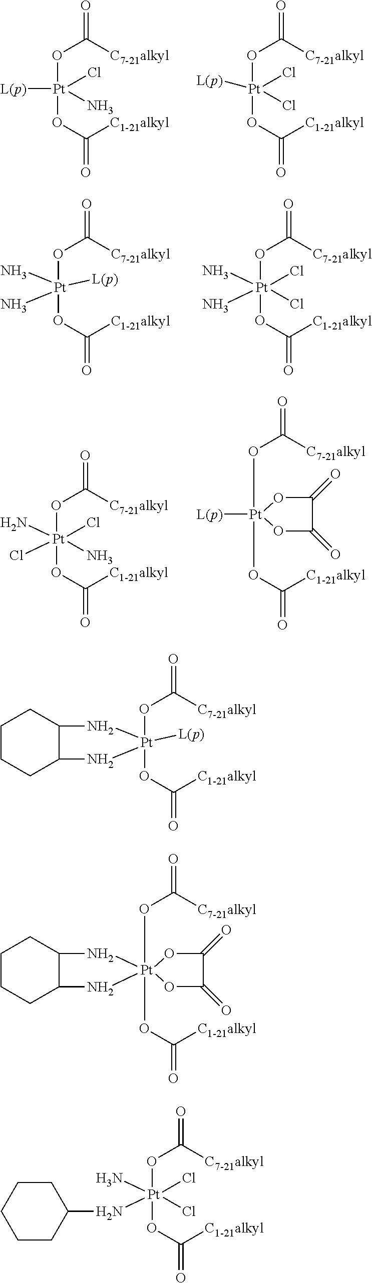

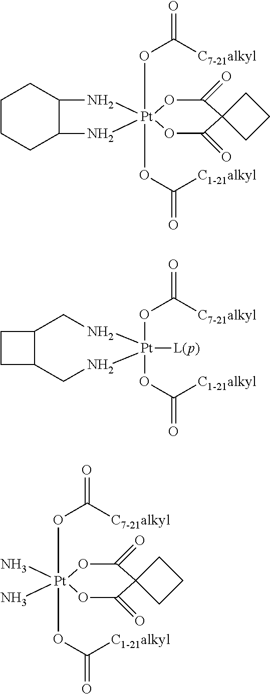

wherein p is independently the number of ligand moieties, being selected from 0, 1 and 2.
being of formula (M10) or (M11):

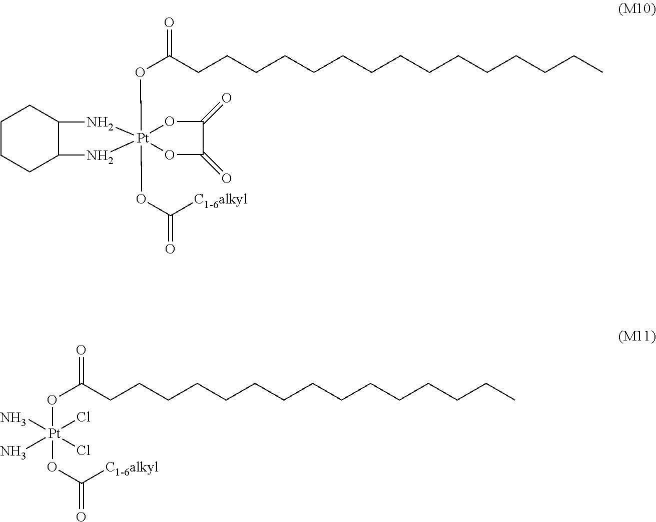

wherein said C₁₋₆alkyl is methyl.
wherein the compound is oxaliplatin palmitate acetate.
being of formula (M12) or (M13):

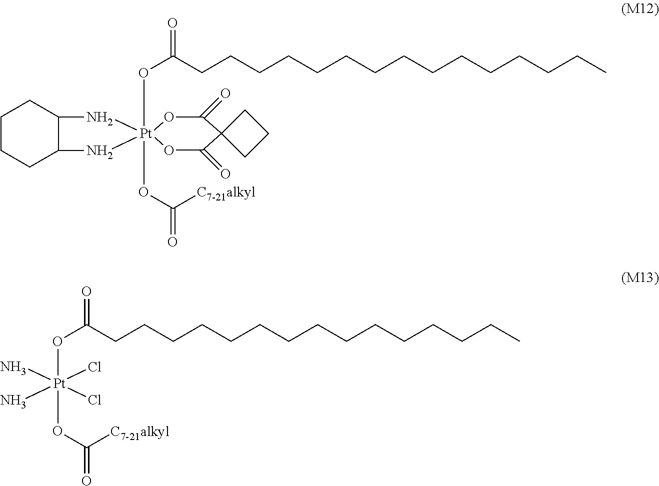

being of formula (M14) or (M15):

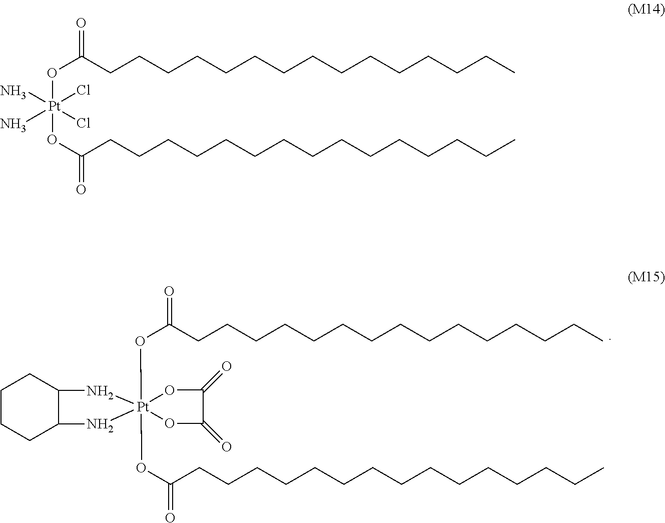

wherein the platinum atom is associated to one or more C₈₋₂₂ fatty acid groups, provided that said fatty acid is not a C₆-C₉ branched alkyl fatty acid.

In some embodiments, compounds of the invention are not compounds disclosed in International Patent Application No. PCT/IL2015/050448 (WO 2015/166498).

In some embodiments, compounds of disclosed in International Patent Application No. PCT/IL2015/050448 (WO 2015/166498) are hereby excluded.

In another aspect, the invention provides a method of preparation of compounds of the invention, the method comprising:
obtaining a Pt complex in its active form (active agent);
reacting said active complex with an oxidizing agent and reactive 4-phenylbutyrate; and
optionally reacting said complex in the presence of a further ligand moiety, being in some embodiments, at least one therapeutically active anticancer moiety or a linker moiety associated with one or more additional Pt active centers;

to thereby obtain the complex of the invention.

In another aspect, the invention provides a method of preparation of compounds of the invention, the method comprising:
obtaining a Pt complex being in its active form (active agent);
oxidizing said active Pt complex to yield an inactive complex form;
obtaining 4-phenylbutyrate in a reactive form;
reacting said inactive complex with said reactive 4-phenylbutyrate, and
optionally reacting said complex in the presence of a further ligand moiety, being in some embodiments, at least one therapeutically active anticancer moiety or a linker moiety associated with one or more additional Pt active centers;
   to thereby obtain the complex of the invention.

The term “Pt complex being in its active form” refers to Pt complex having a therapeutic effect. In some embodiments Pt being in its active form refers to Pt(II) complex.

Typically, oxidation of an active Pt complex is carried out in the presence of an oxidizing agent. In some embodiments, the oxidizing agent is selected from peroxide agents. In some embodiments, the oxidizing agent is an inorganic peroxide or an organic peroxide.

In some embodiments the oxidizing agent is an inorganic peroxide selected from H₂O₂, Na₂O₂, K₂O₂, K₂CO₃.H₂O₂, CaO₂.2H₂O₂, peroxymonosulfuric acid (HOSO₂OOH), peroxydisulfuric acid (HOSO₂OOSO₂OH). In some embodiments, the oxidizing agent is H₂O₂.

In some embodiments the oxidizing agent is an organic peroxide. Non limiting examples of organic peroxide is ROOR or RCOOOCOR or ROOH or RCOOOH or R₃SiOOLi or R₂BOOR or CF₃OOOCF₃, wherein R is an organic moiety, typically selected from alkyl, alkenyl, alkynyl, cycloalkyl, cycloalkenyl, cycloalkynyl, aryl, heteroaryl, heterocyclyl. In some embodiments, the inorganic peroxide is tert-butyl hydrogen peroxide.

In some embodiments the oxidizing agent (in particular peroxides) are presence in a solvent. The solvent may be aqueous solvent or organic solvent. In some embodiments the solvent is water. In some embodiments the solvent is organic solvent. Non-limiting examples of organic solvents are acetonitrile, tetrahydrofuran, dimethyl formamide, ethanol, chloroform, hexane or any combination thereof.

In some embodiments, the oxidizing agent is inorganic peroxide in water. In some embodiments, the oxidizing agent is H₂O₂ in water.

In some embodiments, the oxidizing agent is inorganic peroxide or an organic peroxide in an organic solvent. In some embodiments, the oxidizing agent is H₂O₂ in an organic solvent.

In some embodiments (e.g., when the oxidizing agent is H₂O₂ in water), the oxidizing step results in a Pt complex having dihydroxido species. In some embodiments, the dihydroxido species having axial hydroxides on the Pt complex.

In some embodiments, the axial hydroxides (dihydroxide) form an axially symmetric complex, i.e., a pair of two identical ligands on the axial axis.

In some embodiments (e.g., when the oxidizing agent is organic peroxide in organic solvent or inorganic peroxide in organic solvent), the oxidizing step results in a Pt complex having a single hydroxide (monohydroxido species) or dihydroxide (dihydroxido species).

In some embodiments, the dihydroxido species having axial hydroxides on the Pt complex.

In some embodiments, the monohydroxide or the dihydroxide form a non-symmetric axial complex, i.e., non-identical ligands on the axial axis. The hydroxides on the Pt complex may be reacted with 4-phenylbutirate or reactive 4-phenylbutirate.

The term “reactive 4-phenylbutyrate” or “phenylbutyrate in a reactive form” refers to any form of 4-phenylbutyrate precursor which enables formation of the complex of the invention, when reacted with the Pt complex. In other words, the phenylbutyrate in a reactive form is a phenylbutyrate in the form of a reactant or precursor in the reaction of the inactive Pt complex to form the complex of the invention. In some embodiments, the reactive 4-phenylbutyrate is an anhydride of 4-phenylbutyrate. In some embodiments, the reactive 4-phenylbutyrate is a chloric acid of 4-phenylbutyrate. In some embodiments, the reactive 4-phenylbutyrate is acyl chloride of 4-phenylbutyrate. In some embodiments, the reactive 4-phenylbutyrate is an activated esters of 4-phenylbutyrate. In some embodiments, the reactive 4-phenylbutyrate is an isocynates of 4-phenylbutyrate. In some embodiments, the reactive form of 4-phenylbutyrate is 4-phenylbutyrate.

The reactive 4-phenylbutyrate may be obtained by direct synthesis or from a commercial source. For example, anhydride of 4-phenylbutyrate may be obtained by reacting 4-phenyl butyric acid with N,N′-dicyclohexylcarbodiimide).

In some embodiments (e.g. when obtaining a non-symmetric axial complex of the invention), the step of reacting said inactive complex with said reactive 4-phenylbutyrate refers to reacting a single hydroxide (e.g., carboxylation) and having another ligand (which may be hydroxide, carboxyl group, carbonyl group or any other ligand) subjected to a further/another chemical reaction.

In another aspect, the invention provides a compound of the invention as a prodrug. As used herein the term “prodrug” refers to an agent which is converted into the parent drug (active agent) in vivo by some physiological chemical process (e.g., a prodrug converted to the desired drug form under physiological conditions). The prodrugs of the invention are useful as they may be easier to administer than the parent drug, they are less toxic and present improved bioavailability.

After administration, the prodrug is enzymatically or chemically cleaved to deliver the active drug and the ligand moieties as free molecules in the blood or tissue.

In some embodiments, the prodrug may release at least one active agent. In other embodiments, the prodrug may release one or two or three active agents.

In some embodiments, the prodrug of the invention releases a Pt(II) complex as an active agent. In some embodiments, the prodrug of the invention releases phenyl butyrate or phenyl butyrate derivative (an anion or a salt) as an active agent. In some embodiments, the prodrug of the invention releases Pt(II) complex and phenyl butyrate or phenyl butyrate derivative as active agents.

In some embodiments, the prodrug releases the active agent (activated) in a physiological pH (7.4). In some embodiments, the prodrug is activated at a pH lower than the physiological pH. In some embodiments, the prodrug is activated at a pH of about 6.

A person of skill in the art would realize that a complex of the invention generally releases two (or more) active agent drugs, which may induce, depending on the nature of the ligands, two different mechanisms of action, typically on two different cellular targets. Without wishing to be bound to theory the complex releases Pt(II) active complex which targets DNA molecule. The complex of the invention also releases 4-phenyl butyrate which targets (modify, inhibit) histone deacetylase.

Thus, in another aspect, the invention provides a complex according to the above for targeting DNA and inhibiting histone deacetylase.

In another aspect, the invention provides a pharmaceutical composition comprising at least one compound of any one of the above.

As used herein, “pharmaceutical composition” comprises a therapeutically effective amount of a compound of the present invention, together with suitable diluents, preservatives, solubilizers, emulsifiers, adjuvant and/or carriers. Such compositions are liquids or lyophilized or otherwise dried formulations and include diluents of various buffer content (e.g.; Tris-HCL, acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polglycolic acid, hydrogels, etc, or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils).

In another aspect, the invention provides at least one compound of any one of the above for use in treatment of proliferation disorders.

In a further aspect, the invention provides use of at least one compound of any of the above Formulae for the preparation of a pharmaceutical composition for use in treatment of proliferation disorders.

In yet a further aspect, the invention provides a method for the treatment of a proliferative disorder, the method comprising administering an effective amount of the compound of the invention to a subject suffering from said disorder.

The term “proliferative disorders” encompass diseases or disorders that effect a cellular growth, differentiation or proliferation processes. In some embodiments, the proliferation disorder is cancer. The term “cancer” as used herein encompasses any neoplastic disease which is characterized by abnormal and uncontrolled cell division causing malignant growth or tumor. Cancer as used herein may refer to either a solid tumor or tumor metastasis.

Non-limiting examples of cancer are ovary cancer, and pancreatic cancer, squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer. Solid cancers appear in many forms, for example, breast cancer, prostate cancer, sarcomas, and skin cancer. One form of skin cancer is melanoma.

The term “treatment” as used herein refers to the administering of a therapeutic amount of the composition of the present invention which is effective to ameliorate undesired symptoms associated with a disease, to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease from occurring or a combination of two or more of the above.

The term “effective amount” as used herein is determined by such considerations as may be known in the art. The amount must be effective to achieve the desired therapeutic effect as described above, depending, inter alia, on the type and severity of the disease to be treated and the treatment regime. The effective amount is typically determined in appropriately designed clinical trials (dose range studies) and the person versed in the art will know how to properly conduct such trials in order to determine the effective amount. As generally known, an effective amount depends on a variety of factors including the affinity of the ligand to the receptor, its distribution profile within the body, a variety of pharmacological parameters such as half-life in the body, on undesired side effects, if any, on factors such as age and gender, etc.

In some embodiments, the effective amount of the compound is administrated by one or more of the following routes: oral, rectal, transmucosal, transnasal, intestinal, parenteral, intramuscular, subcutaneous, intramedullary injections, intrathecal, direct intraventricular, intravenous, intraperitoneal, intranasal, or intraocular injections.

The compounds utilized in accordance with the present invention may be used in their free base or free acid form or as “pharmaceutically acceptable salt(s)”, namely as salts that are safe and effective for pharmaceutical use in mammals (e.g., humans) and that possess the desired biological activity.

### BRIEF DESCRIPTION OF THE DRAWINGS

In order to understand the invention and to see how it may be carried out in practice, embodiments will now be described, by way of non-limiting example only, with reference to the accompanying drawings, in which:

FIG. 1 provides a partial list of platinum(IV) complexes with axial valproate or 4-PhB ligands in accordance with the invention.

FIGS. 2A-2B describe cellular and nuclear accumulation of equi-Pt doses (5 μM) incubated with MCF-7 cells for 24 h (FIG. 2A) and platination levels of nuclear DNA following a 24 h incubation of 5 μM of the complexes with MCF-7 cells (FIG. 2B).

FIGS. 3A-3C describe: FIG. 3A—MCF-7 cells were incubated for 24 h with IC₅₀ concentrations of tested complexes. The HDAC activity was determined in cells (light blue bars) or in nuclear extracts (orange bars), *P<0.05, **P<0.01. FIG. 3B—MCF-7 cells were incubated for 24 h with IC₅₀ concentrations of either cisplatin or oxaliplatin and HDAC activity was determined. FIG. 3C—MCF-7 cells were incubated for 24 h with increasing concentrations of tested complexes. HDAC IC₅₀ values were calculated by 4-PL model (P<0.05).

FIGS. 4A-4C describe: FIG. 4A—Nuclear DNA fragmentation. MCF-7 cells were treated for 12 or 24 h with IC₅₀ of I, VI or Cisplatin (CDDP). Quantitative estimation of DNA fragmentation was obtained with an ELISA test. Data are the means of five independent experiments. Error bars indicate SD. FIG. 4B—MCF-7 cells were treated with IC₅₀ of I and VI for 72 h and stained with the fluorescent dye Hoechst 33258. FIG. 4C—Caspase activity. MCF-7 cells incubated for 24 h with IC₅₀ of I, VI or staurosporine±broad-spectrum caspase inhibitor zVAD, and processed for caspase-3/-7, -6, -8, -9 activity. Data are the means of at least three independent experiments. Error bars indicate SD.

FIGS. 5A-5B describes: FIG. 5A—Effects of I or VI on cellular mitochondrial membrane potential. MCF-7 cells were treated for 24 or 48 h with IC₅₀ of I or VI. The percentage of cell with hypopolarized mitochondrial membrane potential was determined by Mito-ID® Membrane Potential Kit. Data are the means of five independent experiments. Error bars indicate SD. FIG. 5B—MCF-7 cells were treated with IC₅₀ of I or VI for 24 h. The amount of p53 was detected by Western blotting analysis as described in the ESI.

FIG. 6 depicts additional compounds of the invention.

FIG. 7 provides ¹H NMR spectra of compound 3a (see Scheme 4) in phosphate buffer.

FIG. 8 provides ¹H NMR spectra of compound 3a (see Scheme 4) in DMEM.

FIG. 9 provides ¹H NMR spectra of compound 3a (see Scheme 4) in the presence of 1 Eq of ascorbic acid.

FIGS. 10A-10B depict cellular uptake and distribution. 2008 (FIG. 10A) and PSN-1 (FIG. 10B) cells were incubated for 24 h with 0.1 μM of tested complexes. The cellular Pt levels in cellular sub-fractions were quantified by means of GF-AAS analysis. Data are the means of at least three independent experiments. Error bars indicate SD.

FIGS. 11A-11B provide: FIG. 11A) Platination levels of nuclear DNA extracts. 2008 and PSN-1 cells were treated for 24 h with 0.1 μM of tested complexes. DNA was extracted, quantifies and the amount of Pt bound to DNA was estimated by GF-AAS. Data are the means of at least three independent experiments. Error bars indicate SD. FIG. 11B) HDAC inhibition. 2008 and PSN-1 cells were incubated for 24 h with IC₅₀ concentrations of tested complexes. The HDAC activity was determined in cells by FLUOR DE LYS® HDAC fluorometric activity assay kit (Enzo Life Sciences) following the manufacturers' instructions. Data are the means of at least three independent experiments. Error bars indicate SD.

FIGS. 12A-12C provide: FIG. 12A) Effects on cellular mitochondrial membrane potential. PSN-1 cells were treated for 12 h with IC₅₀ of tested compounds. The percentage of cells with hypopolarized mitochondrial membrane potential was determined by Mito-ID® Membrane Potential Kit. Data are the means of three independent experiments. Error bars indicate SD. FIG. 12B) Effects on glycolysis. PSN-1 cells were treated for 24 h with IC₅₀ of tested compounds. Than cells were stained with the Mito-ID® 02 Sensor Probe Solution. Fluorescence was estimated at 350 nm (excitation) and 610 nm (emission). Data are the means of three independent experiments. Error bars indicate SD. FIG. 12C) TEM analysis. Micrographs of PSN-1 cells after 48 h of treatment with tested complexes. (a) control; (b) 3a; (c) 3b.

FIGS. 13A-13B provide: FIG. 13A) Cytochrome c release. PSN-1 cells were treated for 12 or 24 h with IC₅₀ of 3a and cytochrome c was estimated by Western blotting. FIG. 13B) Nuclear DNA fragmentation. PSN-1 cells were treated for 24 or 48 h with IC₅₀ of 3a or cisplatin (CDDP). Quantitative estimation of DNA fragmentation was obtained with an ELISA test. Data are the means of five independent experiments. Error bars indicate SD.

### DETAILED DESCRIPTION OF EMBODIMENTS

Synthesis of the Compounds of the Invention:

Cisplatin and oxaliplatin are commercially available and can be easily synthesized in the lab. 4-phenybutyrate is also commercially available.

Cisplatin or oxaliplatin were oxidized with H₂O₂ to yield the Pt(IV) complexes, as shown in Scheme 1:

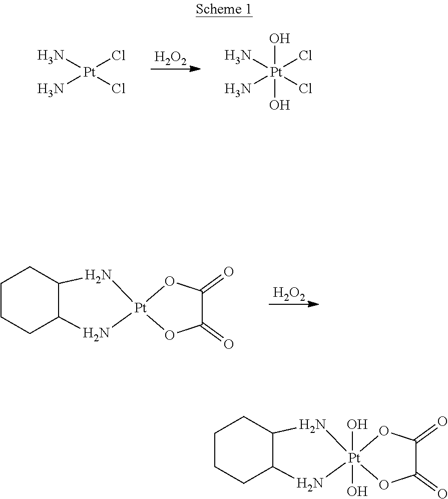

The anhydride of 4-phenybutyrate was prepared by standard methods, as shown in Scheme 2: 4-phenyl butyric acid (1 eq.) and N,N′-Dicyclohexyl carbodiimide (DCC, 0.5 eq) were dissolved in Chloroform (15 ml) and stirred at room temperature overnight. The dicyclohexylurea byproduct was removed by celite filtration. The crude mixture was re-dissolved in DCM (30 mL), concentrated and filtered, and was repeated until no urea was observed.

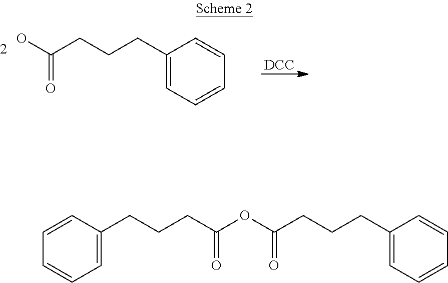

The final compound were obtained by reacting the Pt(IV) dihydroxido complexes with the anhydride, as shown in Scheme 3:

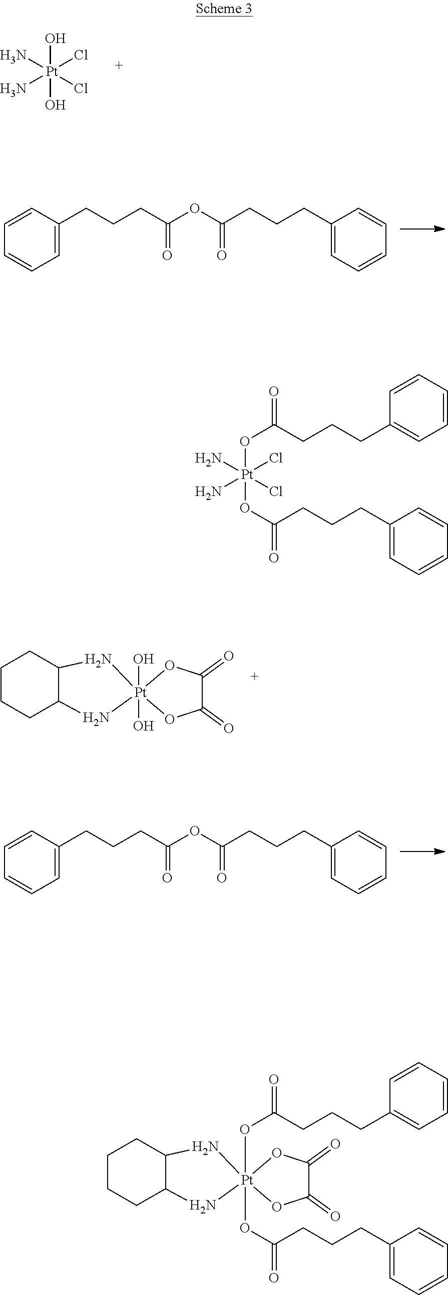

The final compounds were purified by HPLC and characterized by NMR spectroscopy and elemental analysis.

### EXPERIMENTAL

NMR Spectroscopy:

¹H NMR and ¹H NMR data were recorded on Varian Unity-500 MHz and Varian Unity-300 MHz spectrometers respectively and Data were processed using MestreNova software.

The purities of tested compound are >95% (HPLC). HPLC analyses were performed in a Varian ProStar HPLC system equipped with a UV detector, set at 220 nm, using a RP-C18 column (Phenomenex, Luna, 250×4.6 mm, 5 μm, 100 Å).

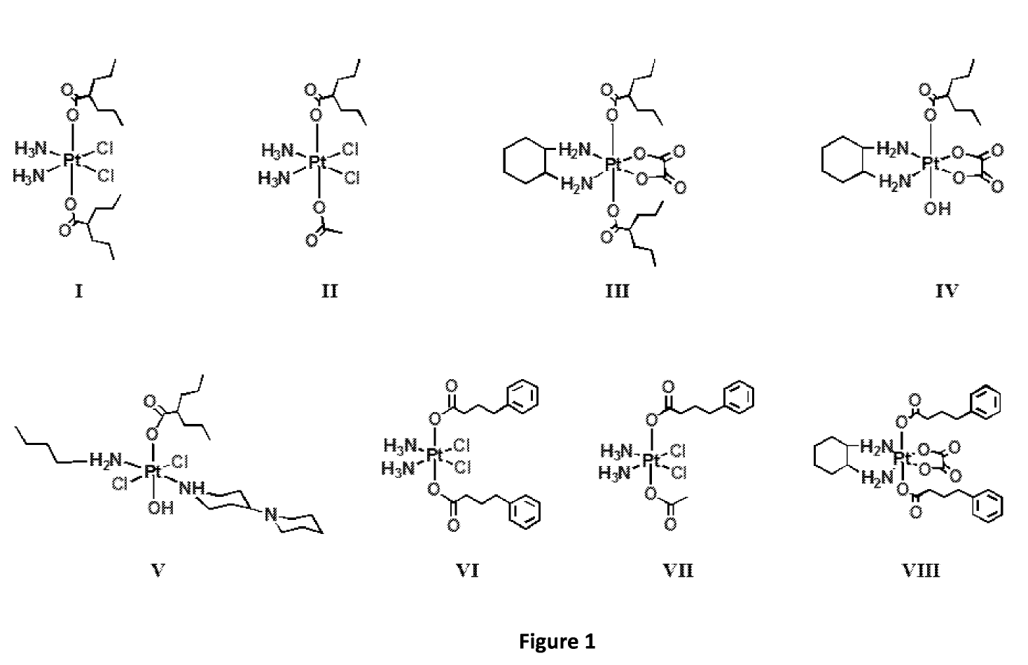

Synthesis of compounds (see FIG. 1; all compounds were prepared following a general procedure).

Ctc-Pt(NH₃)₂(C₈H₁₅O₂)₂Cl₂] (1)

Starting material [Pt(NH₃)₂(OH)₂Cl₂] was obtained by H₂O₂ oxidation of cisplatin.

[Pt(NH₃)₂(OH)₂Cl₂] (50 mg, 1.49×10⁻⁴ mol) was suspended in N,N-dimethylformamide (1 mL) and valproic anhydride was added (101.18 mg, 3.74×10⁻⁴ mol, 2.5 eq.). The reaction mixture was stirred for 5 h (complete dissolution) at 40° C. Solvent was evaporated at the end of the reaction and diethyl ether was added to precipitate the desired compound as a light yellow powder. The precipitate was separated by centrifugation and washed twice with ethyl ether and dried in vacuo.

Yield: 71 mg, 77.2%, ¹⁹⁵Pt-NMR (DMF): 1174 ppm.

¹H-NMR (DMSO-d⁶): δ ppm 1.19 (t, 12H), 1.49-1.89 (m, 16H), 2.6 (m, 2H), 6.51-6.98 (b, 6H).

Pt(NH₃)₂Cl₂(C₂H₃O₂)(C₈H₁₅O₂)] (II)

Starting material [Pt(NH₃)₂(OH) (C₂H₃O₂) Cl₂] was prepared by oxidizing cisplatin in acetic acid.

To a solution of [Pt(NH₃)₂(OH) (C₂H₃O₂) Cl₂] (50 mg, 1.32×10⁻⁴ mol) in N,N-dimethylformamide (2 mL), valproic anhydride (53.92 mg, 1.96×10⁻⁴ mol, 1.5 eq.) was added. After stirring for 2 h at rt, solvent was evaporated at reduced pressure. Acetone was added to the residue and the precipitated compound was collected by filtration, washed twice with acetone, and dried in vacuo to afford the desired compound as a light yellow powder.

Yield: 59 mg, 88%. ¹⁹⁵Pt-NMR (DMF):1177 ppm.

¹H-NMR (DMSO): δ ppm 0.89 (t, 6H), 1.13-1.49 (m, 8H), 1.89 (s, 3H), 6.51-6.90 (b, 6H).

[Pt(DACH)(C₈H₁₅O₂)₂(ox)] (III)

The starting material, [Pt(DACH)(OH)₂(ox)] was prepared according to Hall et al. (J Biol Inorg Chem. 2003 September; 8(7):726-32.)

To a suspension of [Pt(DACH) (OH)₂ (ox)] (50 mg, 1.16×10⁻⁴ mol) in N,N-dimethylformamide (1 mL), valproic anhydride (78.37 mg, 2.88×10⁻⁴ mol, 2.5 eq) was added. The mixture was stirred at rt for 12 h. Solvent was evaporated at the end of the reaction and the residue was redissolved in 5 mL dichloromethane. Diethyl ether was added to precipitate the desired compound as a white powder. The precipitate was separated by centrifugation, washed twice with diethyl ether and dried under vacuum.

Yield: 68 mg, 86%

¹⁹⁵Pt-NMR (DMF):1569 ppm. ¹H-NMR (DMSO): δ ppm 1.10 (t, 12H), 1.30-1.90 (m, 24H), 2.4-2.8 (m, 4H), 8.74-9.09 (b, 4H).

[Pt(DACH)(C₈H₁₅O₂)(OH)(ox)] (IV)

Oxaliplatin diOH (10 mg, 2.3×10-5 mol) and Valproic anhydride (70 uL, 2.3×10-4 mol) were dissolved in DMF (2 mL) and stirred at RT overnight.

DMF was evaporated. The residue was dissolved in DCM and ethyl ether was added to induce precipitation. The white precipitated was collected by centrifuge and washed with ether. Remaining ether was evaporated under a nitrogen stream. Yield: 81%, 10.4 mg, 1.86×10-5 mol.

ctc-[Pt(NH₃)₂(C₁₀H₁₁O₂)₂Cl₂] (VI)

[Pt(NH₃)₂(OH)₂Cl₂] (100 mg, 2.99×10⁻⁴ mol) was suspended in N,N-dimethylformamide (1 mL) and 4phenylbutiric anhydride was added (232.28 mg, 7.47×10⁻⁴ mol, 2.5 eq). The reaction mixture was stirred for 1 h (complete dissolution) at 40° C. The solvent was evaporated at reduced pressure. The residue was redissolved in acetone and diethyl ether was added to precipitate the compound. The precipitate was collected by filtration, washed twice with diethyl ether and dried in vacuo.

Yield: 138 mg, 74%

¹⁹⁵Pt-NMR (DMF):1174 ppm. ¹H-NMR (DMSO): δ ppm 1.72 (m, 4H), 2.20 (t, 4H), 2.57 (t, 4H), 6.27-6.80 (b, 6H), 7.24 (m, 10H).

Ctc-[Pt(NH₃)₂(C₂H₃O₂)(C₁₀H₁₁O₂)Cl₂] (VII)

To a solution of [Pt(NH₃)₂(OH) (C₂H₃O₂) Cl₂] (50 mg, 1.32×10⁻⁴ mol) in N,N-dimethylformamide (2 mL), 4phenylbutirc anhydride (61.89 mg, 1.99×10⁻⁴ mol, 1.5 eq) was added. After stirring for 1 h at rt, solvent was evaporated at reduced pressure. Acetone was added to the residue and the precipitated compound was collected by filtration, washed twice with acetone, and dried in vacuo to afford the desired compound as a light yellow powder.

Yield: 60 mg, 87%

¹⁹⁵Pt-NMR(DMF):1176 PPM, ¹H-NMR (DMSO): δ ppm 1.704 (m, 2H), 1.87 (s, 3H), 2.18 (t, 2H), 2.55 (t, 2H), 6.5 (b, 6H), 7.18 (m, 5H).

[Pt(DACH)(C₁₀H₁₁O₂)₂(ox)] (VIII)

To a suspension of [Pt(DACH) (OH)₂ (ox)] (100 mg, 2.32×10⁻⁴ mol) in N,N-dimethylformamide (1 mL), 4phenylbutyric anhydride (179.91 mg, 5.795×10⁻⁴ mol, 2.5 eq) was added. The mixture was stirred at RT for 2.5 h. Solvent was evaporated at the end of the reaction and dichloromethane was added to precipitate the desired compound as a white powder. The compound was separated by centrifugation, washed twice with dichloromethane and dried under vacuo.

Yield: 121 mg, 72%

¹⁹⁵Pt-NMR(DMF):1588 PPM. ¹H-NMR (DMSO): δ ppm 1.0-1.59 (m, 8H), 1.69 (m, 4H), 2.02-2.34 (m, 8H), 7.2 (m, 10H), 8.32 (b, 4H).

Experiments with Cultured Human Cells.

Platinum(IV) complexes were dissolved in DMSO to stock solutions of 1 mg/mL just before the experiment, and a calculated amount of drug solution was added to the cell growth medium to a final solvent concentration of 0.5%, which had no discernible effect on cell killing. Cisplatin (CDDP) and oxaliplatin (OXP) were dissolved just before the experiment in a 0.9% NaCl solution.

Cisplatin, MTT (3-(4,5-dimethylthiazol-2-yl)-2,5-diphenyltetrazolium bromide), 4-phenylbutyric acid, staurosporin, Z-VAD-fmk and Hoechst 33258 (2′-(4-hydroxyphenyl)-5-(4-methyl-1-piperazinyl)-2,5′-bi-1H-benzimidazole trihydrochloride hydrate) were obtained from Sigma Chemical Co, St. Louis, USA.

Cell Cultures.

Human lung (A549), breast (MCF-7), pancreatic (BxPC3), kidney (A498) and colon (HCT-15) carcinoma cell lines along with melanoma (A375) were obtained from American Type Culture Collection (ATCC, Rockville, Md.). Human prostate adenocarcinoma (PC3) cells were obtained from European Collection of Cell Cultures (ECACC, Salisbury, UK). Human ovarian cancer cell lines A2780 and its cisplatin resistant variant, A2780cisR, were kindly provided by Prof. G. Marverti (Dept. of Biomedical Science of Modena University, Italy. Cell lines were maintained in the logarithmic phase at 37° C. in a 5% carbon dioxide atmosphere using the following culture media containing 10% fetal calf serum (Euroclone, Milan, Italy), antibiotics (50 units·mL⁻¹ penicillin and 50 μg·mL⁻1 streptomycin) and 2 mM 1-glutamine: i) RPMI-1640 medium (Euroclone) for MCF-7, HCT-15, A431, BxPC3, A2780 and A2780 cisR cells; ii) F-12 HAM'S (Sigma Chemical Co.) for A549 and PC3 cells; iii) D-MEM medium (Euroclone) for A375 cells; iv) EMEM for A498 cells.

Cytotoxicity MTT Assay.

The growth inhibitory effect towards human cell lines was evaluated by means of MTT (tetrazolium salt reduction) assay [Alley, M. C.; Scudiero, D. A.; Monks, A.; Hursey, M. L.; Czerwinski, M. J.; Fine, D. L.; Abbott, B. J.; Mayo, J. G.; Shoemaker, R. H.; Boyd, M. R. Feasibility of drug screening with panels of human tumor cell lines using a microculture tetrazolium assay. Cancer Res. 1988, 48, 589-601]. Briefly, 3-8.10³ cells/well, dependent upon the growth characteristics of the cell line, were seeded in 96-well microplates in growth medium (100 μL) and then incubated at 37° C. in a 5% carbon dioxide atmosphere. After 24 h, the medium was removed and replaced with a fresh one containing the compound to be studied at the appropriate concentration. Triplicate cultures were established for each treatment. After 72 h, each well was treated with 10 μL of a 5 mg·mL⁻¹ MTT (3-(4,5-dimethylthiazol-2-yl)-2,5-diphenyltetrazolium bromide) saline solution, and after 5 h additional incubation, 100 μL of a sodium dodecylsulfate (SDS) solution in HCl 0.01 M were added. After overnight incubation, the inhibition of cell growth induced by the tested complexes was detected by measuring the absorbance of each well at 570 nm using a Bio-Rad 680 microplate reader (Bio-Rad, Hercules, Calif.). Mean absorbance for each drug dose was expressed as a percentage of the control untreated well absorbance and plotted vs drug concentration. IC₅₀ values represent the drug concentrations that reduced the mean absorbance at 570 nm to 50% of those in the untreated control wells.

Cellular Accumulation.

MCF-7 cells (2.5×10⁶) were seeded in 75 cm² flasks in growth medium (20 ml). After overnight incubation, the medium was replaced and the cells were treated with tested compounds for 24 h. Cell monolayers were washed twice with cold PBS, harvested and counted. Cell nuclei were isolated by means of the nuclei isolation kit Nuclei EZ Prep (Sigma Co.). Samples were than subjected to three freezing/thawing cycles at −80° C., and then vigorously vortexed. The samples were treated with highly pure nitric acid (Pt: ≤0.01 μg kg⁻¹, TraceSELECT® Ultra, Sigma Chemical Co.) and transferred into a microwave teflon vessel. Subsequently, samples were submitted to standard procedures using a speed wave MWS-3 Berghof instrument (Eningen, Germany) After cooling, each mineralized sample was analyzed for platinum by using a Varian AA Duo graphite furnace atomic absorption spectrometer (Varian, Palo Alto, Calif.; USA) at the wavelength of 324.7 nm. The calibration curve was obtained using known concentrations of standard solutions purchased from Sigma Chemical Co. purchased from Sigma Chemical Co.

DNA platination. MCF-7 cells (3×10⁶) were seeded in 10 cm Petri dishes in 10 mL of culture medium. Subsequently, cells were treated with tested complexes for 24 h. DNA was extracted and purified by a commercial spin column quantification kit (Qiagen DNeasy Blood and Tissue Kit). Only highly purified samples (A260/A230≅1.8 and A280/A260≅2.0) were included for analysis to avoid any artifacts. The samples were completely dried and re-dissolved in 200 μL of Milli-Q water (18.2 MΩ) for at least 20 min at 65° C. in a shaking thermo-mixer, mineralized and analyzed for total Pt content by GF-AAS as described above.

Histone Deacetylase Assay.

Histone deacetylase activity was determined using Fluor-de-Lys® HDAC fluorometric activity assay kit (Enzo Life Sciences International, Inc., Plymount Meeting, Pa., U.S.A.). MCF-7 cells (5.10⁴ seeded in 96-well microplates) were treated for 24 h with equi-toxic concentrations of tested complexes, corresponding to the IC₅₀ values, and then processed as reported by the manufacturer's instructions. Fluorescence was measured using a Fluoroskan Ascent FL (Labsystem, Finland) plate reader, with excitation at 360 nm and emission at 460 nm. For comparison purpose, the HDAC activity was also measured in nuclear extracts of MCF-7-treated cells by means of the same fluorometric activity assay kit. In this latter case, MCF-7 nuclear extracts were obtained as follow: cell pellets were re-suspended in lysis buffer [Tris.HCl (10 mM, pH 8.0), KCl (60 mM), EDTA (1.2 mM), DTT (1 mM), PMSF (0.05 mM), NP-40 (0.05%)] and kept on ice for 10 min. Subsequently, samples were centrifuged at 1000 g and re-suspended in the nuclear extraction buffer [Tris.HCl (20 mM, (pH 8.0), NaCl (420 mM), MgCl₂ (0.7 mM), EDTA (0.25 mM), glycerol (25%)] for 30 min at 4° C., and then centrifuged at 15000 g for 15 min at 4° C.

Caspase-3/-7 Activation.

Caspase-3/-7 activity was detected by using the Apo-One® 3/7 Homogeneous Caspase-3/7 Assay (Promega, Madison, Wis., USA) according to the manufacturer's recommended procedures. MCF-7 cells were seeded in 96-well microplates in growth medium (100 μL) and then incubated at 37° C. in a 5% carbon dioxide atmosphere. After overnight incubation, cells were subjected to a 24 h treatment with tested compounds (at concentrations corresponding to IC₅₀ values). Subsequently, each well was treated with 120 μl of the Apo-ONE® Caspase-3/7 Assay Reagent containing the specific substrate (rhodamine 110 bis-(N-CBZ-1-aspartyl-1-glutamyl-1-valyl-aspartic acid amide), Z-DEVD-R110). The fluorescence was determined after one hour with a PerkinElmer 550 spectrofluorometer (excitation 499 nm, emission 521 nm).

Hoechst 33258 Staining.

MCF-7 cells were seeded into 8-well tissue-culture slides (BD Falcon, Bedford, Mass., USA) at 5.10⁴ cells/well (0.8 cm²). After 24 h, cells were washed twice with PBS and following 72 h of treatment with IC₅₀ concentration of tested complexes, cells were stained for 5 min with 1 mg/mL Hoechst 33258 (Sigma-Aldrich) in PBS before being examined by fluorescence microscopy (Olympus BX41, Cell F software, Olympus, Munster, Germany)

A group of the compounds synthesized for this study are depicted in FIG. 1. Generally, as stated above, the synthesis of the symmetric compounds I, IV, VI and VIII was done by standard procedures. In brief, cisplatin or oxaliplatin were oxidized with H₂O₂ to yield ctc-[Pt(NH₃)₂(OH)₂Cl₂] or [Pt(DACH)(OH)₂(ox)] that were then reacted with an excess of the anhydrides of valproic acid or of 4-phenylbutyric acid to yield the desired compounds.

The non-symmetric compound ctc-[Pt(NH₃)₂(OH)(OAc)Cl₂] was prepared by oxidizing cisplatin in acetic acid. It was then reacted with 1.5 equivalents of the appropriate anhydride in DMF to yield ctc-[Pt(NH₃)₂(L)(OAc)Cl₂] where L=VPA, PhB.

All compounds were characterized by ¹H NMR spectroscopy and when possible by mass spectrometry. They were purified by preparative HPLC and their purity was ascertained by elemental analysis.

### Cytotoxicity Data:

Compounds I-VIII, the corresponding uncoordinated axial ligands (VPA and PhB) as well as cisplatin and oxaliplatin were screened against seven different cancer cell lines representative of lung (A549), breast (MCF-7), pancreatic (BxPC3), kidney (A498) and colon (HCT-15) carcinoma, along with melanoma (A375). Furthermore, Pt(IV) complexes were also tested for their in vitro cytotoxicity on a pair of human ovarian adenocarcinoma cell lines which have been selected for sensitivity/resistance to cisplatin (cancer cells A2780/A2780cisR). The cytotoxicity parameters, in terms of IC₅₀ (the median growth inhibitory concentration calculated from dose-survival curves) obtained after 72 h exposure, are reported in Table 1 below.

TABLE 1
cytotoxicity parameters, in terms of IC₅₀ (the median growth inhibitory concentration calculated
from dose-survival curves) measured after 72 h exposure for compounds of the invention.

| Cell line | CDDP | OXP | I | II | III | IV |
|---|---|---|---|---|---|---|
| A2780 | 2.8 ± 0.2 | 0.52 ± 0.27 | 0.25 ± 0.09 | 1.02 ± 0.6 | 1.3 ± 0.2 | 5.4 ± 0.2 |
| A2780cisR | 13.8 ± 0.2 | 4.2 ± 1.6 | 0.30 ± 0.10 | 1.62 ± 0.30 | 8.8 ± 2.1 | 16.1 ± 0.7 |
| MCF-7 | 17.4 ± 0.2 | 3.36 ± 0.2 | 0.91 ± 0.30 | 5.74 ± 1.10 | 5.7 ± 0.9 | 31.6 ± 2.1 |
| A549 | 8.35 ± 0.87 | 1.46 ± 0.31 | 1.12 ± 0.32 | 8.36 ± 1.14 | 20.5 ± 2.14 | 60.3 ± 3.34 |
| HCT-15 | 11.32 ± 1.06 | 1.15 ± 0.43 | 0.81 ± 0.12 | 6.36 ± 1.03 | 16.4 ± 1.63 | 51.5 ± 3.26 |
| PC3 | 2.25 ± 0.82 | 5.24 ± 0.86 | 2.13 ± 0.45 | 7.32 ± 1.24 | 10.1 ± 1.17 | 53.2 ± 4.24 |
| A498 | 17.53 ± 1.21 | 7.94 ± 1.04 | 2.43 ± 0.72 | 4.21 ± 0.95 | 5.21 ± 1.10 | 21.1 ± 3.07 |
| BxPC3 | 11.36 ± 1.14 | 4.15 ± 0.93 | 1.13 ± 0.14 | 3.54 ± 0.66 | 14.4 ± 2.16 | 19.8 ± 2.25 |
| A375 | 4.03 ± 0.95 | 6.30 ± 1.11 | 1.25 ± 0.25 | 2.17 ± 0.53 | 4.17 ± 0.87 | 47.3 ± 2.53 |

| Cell line | V | VI | VII | VIII | 4-PB |
|---|---|---|---|---|---|
| A2780 | 1.18 ± 0.33 | 0.14 ± 0.03 | 0.65 ± 0.04 | 0.90 ± 0.08 | — |
| A2780cisR | 1.29 ± 0.29 | 0.12 ± 0.03 | 1.54 ± 0.27 | 1.96 ± 0.44 | — |
| MCF-7 | 8.80 ± 1.48 | 0.18 ± 0.03 | 3.43 ± 0.14 | 2.28 ± 0.46 | 1735 ± 27 |
| A549 | 15.5 ± 1.13 | 0.17 ± 0.04 | 7.16 ± 1.11 | 19.63 ± 3.22 | 1989 ± 22 |
| HCT-15 | 14.4 ± 2.05 | 0.31 ± 0.04 | 5.17 ± 0.85 | 12.54 ± 1.13 | — |
| PC3 | 16.8 ± 1.41 | 0.41 ± 0.06 | 4.34 ± 1.25 | 6.35 ± 1.19 | 795 ± 18 |
| A498 | 8.64 ± 0.95 | 0.43 ± 0.02 | 8.42 ± 1.47 | 9.32 ± 1.07 | — |
| BxPC3 | 4.43 ± 1.94 | 0.19 ± 0.01 | 10.14 ± 1.69 | 7.64 ± 1.15 | 1523 ± 36 |
| A375 | 12.4 ± 2.93 | 0.16 ± 0.02 | 6.25 ± 1.94 | 0.64 ± 0.13 | |

Uncoordinated HDAC inhibitor ligands VPA and PhB displayed very low cytotoxic activities, with IC₅₀ values in the millimolar range, as already reported in the literature. Mono- and bis-VPA Pt(IV) derivatives of cisplatin showed cytotoxic activities significantly higher than those elicited by cisplatin against all tested cell lines, having average IC₅₀ values (μM), for the nine cancer cell lines, of 1.15 for ctc-[Pt(NH₃)₂(VPA)₂Cl₂], 4.48 for ctc-[Pt(NH₃)₂(VPA)(OAc)Cl₂] and 9.18 for cisplatin. These results, in accordance with the results reported in the literature, clearly indicate that Pt(IV) pro-drugs with bioactive VPA ligands are significantly more potent than the parent cisplatin. In particular, ctc-[Pt(NH₃)₂(VPA)₂Cl₂] was about 19, 14 and 10 times more effective than cisplatin against breast (MCF-7), colon (HCT-15) and pancreatic (BxPC3) cancer cells, respectively. In the sensitive and resistant ovarian cancer cells A2780 and A2780cisR, ctc-[Pt(NH₃)₂(VPA)₂Cl₂] is 11 and 46 fold more potent than cisplatin, with the resistance factor (being the ratio between IC₅₀ values calculated for the resistant cells and those obtained with the sensitive ones) of 1.2, indicating its ability to circumvent acquired resistance to cisplatin.

Interestingly, when one VPA ligand of ctc-[Pt(NH₃)₂(VPA)₂Cl₂] is replaced with an acetato to give ctc-[Pt(NH₃)₂(VPA)(OAc)Cl₂], there is a 1.7-7.9 fold reduction in the potency compared with ctc-[Pt(NH₃)₂(VPA)₂Cl₂] and on the average of the nine cell lines, the bis-VPA derivative of cisplatin was 4.6 fold more potent than the mono-VPA monoacetato analog. Yet, with the exception of the PC3 cells, ctc-[Pt(NH₃)₂(VPA)(OAc)Cl₂] was as or more potent than cisplatin in all tested cells lines and showed the ability to overcome acquired cisplatin resistance in A2780cisR cells (resistance factor of 1.6).

Conversely, the behavior of the Pt(IV) oxaliplatin derivatives with VPA axial ligands was very different than that observed for the Pt(IV) cisplatin derivatives with VPA ligands. The antiproliferative potencies of compounds III, [Pt(DACH)(VPA)₂(ox)] and IV, [Pt(DACH)(VPA)(OH)(ox)], were significantly lower than those of the parent Pt(II) complex oxaliplatin, having average IC₅₀ values (μM) over the nine cell lines of 4.9, 9.62 and 37.6 for oxaliplatin, III and IV, respectively. Moreover, both complexes showed cross resistance with cisplatin in the A2780cisR cells (resistance factor of 6.8 and 3.0 for III and IV, respectively).

The transplatinum derivative (IV), trans-[Pt(n-butylamine)(VPA)(OH) (piperidine) Cl₂], which possesses one VPA ligand in axial position, elicited an average IC₅₀ value over the nine tested cell lines that is very similar to that of cisplatin (9.27 μM). However, it retained a better in vitro antitumor potency with respect to cisplatin against five out of the nine cell lines tested (A2780, A2780cisR, MCF-7, A549, A498 and BxPC3 cells). Notably it was roughly 11 times more effective than cisplatin against the ovarian cancer cell lines A2780cisR, eliciting a resistance factor of 1.1, thus attesting the absence of cross-resistance with cisplatin.

Compounds VI and VII, the Pt(IV) derivatives of cisplatin with either two or one 4-phenylbutyrate (PhB) ligands in the axial positions, showed a significant antiproliferative activity against all tested cell lines. Like with mono- and bis-VPA cisplatin derivatives, the bis-PhB cisplatin derivatives VI and VII are significantly more potent than cisplatin, with average IC₅₀ values (μM) of 0.23 (0.12-0.43) and 4.8 (0.65-10.14) respectively. Remarkably, the bis-PhB derivative, ctc-[Pt(NH₃)₂(PhB)₂Cl₂] (compound VI) was nearly 100-, 60- and 50-fold more potent than cisplatin against MCF-7, BxPC3 and A549 cells, respectively. Furthermore, it showed similar cytotoxicity against the cisplatin sensitive (0.14 μM) and resistant ovarian cancer cells (0.12 μM), denoting its ability to overcome cisplatin resistance.

As in the case of ctc-[Pt(NH₃)₂(VPA)₂Cl₂] and ctc-[Pt(NH₃)₂(VPA)(OAc)Cl₂], where the symmetric bis-VPA complex was significantly more potent than the non-symmetric mono-VPA complex, the symmetric ctc-[Pt(NH₃)₂(PhB)₂Cl₂] was significantly more effective than ctc-[Pt(NH₃)₂(PhB)(OAc)Cl₂]. The bis-PhB is 4.6-53 fold more potent than the non-symmetric mono-PhB monoacetate complex and on the average about 24 fold more potent than cisplatin. Ctc-[Pt(NH₃)₂(PhB)(OAc)Cl₂] was more potent than cisplatin in five out of the nine cells lines by factors of 2.1-9. Replacing a PhB in ctc-[Pt(NH₃)₂(PhB)(OAc)Cl₂] with an acetato ligand significantly diminished the potency of the Pt(IV) complex.

By comparing the cytotoxicity results of I and VI, it is evident that replacing both VPA ligands with PhB in the Pt(IV) derivatives of cisplatin increases the potency of the complex (on the average there is nearly a 5 fold improvement in potency) whereas when the VPA in ctc-[Pt(NH₃)₂(VPA)(OAc)Cl₂] was replaced with PhB, ctc-[Pt(NH₃)₂(PhB)(OAc)Cl₂] and ctc-[Pt(NH₃)₂(VPA)(OAc)Cl₂], elicited a similar cytotoxicity profile.

The Pt(IV) derivative of oxaliplatin, with two PhB, [Pt(DACH)(PhB)₂(ox)] was significantly (4-40 fold) less potent than its cisplatin analog (compound VI) and had similar potency to its VPA analog (compound III).

Cellular Accumulation and Binding to Nuclear DNA

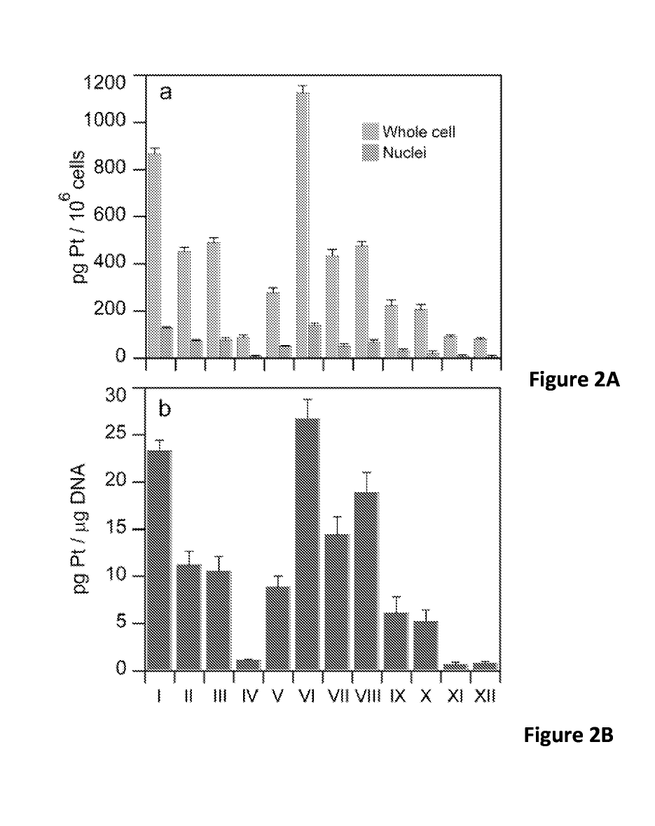

With the aim of identifying a possible correlation between cytotoxic activity and cellular uptake, the cellular Pt content was measured in MCF-7 cells treated for 24 h with equi-Pt concentrations (5 μM) of the Pt(IV) compounds. The intracellular platinum levels were quantified by means of GF-AAS analysis, and the results expressed as pg Pt/10⁶ cells, are shown in FIG. 2. Since platinum drugs are believed to trigger cell death by modifying the nuclear DNA and since co-administration with HDAC inhibitors is supposed to facilitate binding of platinum drugs to DNA by increasing the exposed area of nuclear DNA, we also measured the nuclear accumulation of the complexes and the amount of platinum bound to nuclear DNA, to see whether either of them correlates with cytotoxicity.

Interestingly, the levels of cell associated platinum depend strictly on the structures of the Pt(IV) complexes. Compound VI accumulated most efficiently whereas compound IV was not internalized nearly as efficiently by MCF-7 cells.

The internalization pattern was VI>I>III≈VI>II≈VII>V>IV (see FIG. 1). Overall, uptake results suggest that the Pt(IV) derivatives of cisplatin with two HDACis in the axial positions accumulated most efficiently where the bis-PhB derivative accumulated somewhat more efficiently than its bis-VPA counterpart. The Pt(IV) derivatives of cisplatin with one acetate and either one VPA or one PhB HDACi had similar uptake by the cancer cells. In a similar manner, bis-HDACi oxaliplatin derivatives were equally able to cross cellular cytoplasmic membrane. Interestingly, in this cell line the transpaltinum complex with one VPA ligand also accumulates very efficiently.

Although accumulation of the platinum based drugs in the cancer cell is an essential step in the cascade of events leading to the death of the cancer cells, it does not necessarily correlate with cytotoxicity. It is believed that for platinum drugs the binding to nuclear DNA is the critical step and hence it is important to quantify the levels of nuclear DNA and DNA-bounded platinum. As with whole cell uptake experiments, the highest nuclear Pt accumulation and DNA platination levels were reached with compound VI whereas the lowest values were detected for complex IV. Notably, for all tested complexes the intranuclear accumulation and DNA platination profiles were comparable suggesting that nuclear internalization correlates with DNA-platination.

In addition the amount of platinum was also measured in the cytoplasm. These experiments were performed in two cell lines: MCF-7 (breast cancer) and A375 (melanoma).

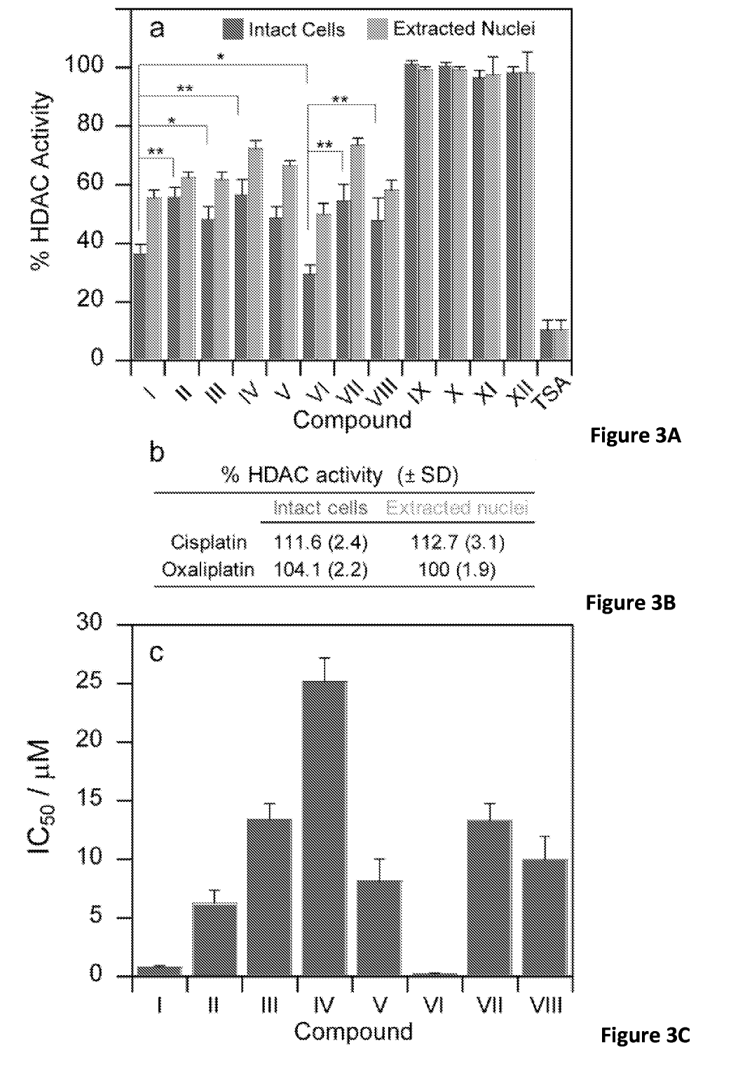

The cells were incubated for 24 h either with equal concentrations of the complexes (FIG. 3) or with equitoxic (IC₅₀) concentration (FIG. 2).

The log P of compounds VI and VIII was measured to see if a correlation existed between lipophilicity and cell association. To quantify the hydrophobicity of the platinum complexes, partition coefficients (log P) values for octanol/water partition were measured using the shake-flask method. The log P values for cisplatin and several compounds I, II, III, VI and VIII are shown in the Table 2 below:

TABLE 2
Log P values for cisplatin and several compounds
I, II, III, VI and VIII shown in FIG. 1.

| Compound Formula | Log P |
|---|---|
| Cisplatin | −2.25 ± 0.04 |
| ctc-[Pt(NH₃)₂(VPA)₂Cl₂] | 0.25 ± 0.03 |
| ctc-[Pt(NH₃)₂(Ac)(VPA) Cl₂] | 0.17 ± 0.02 |
| [Pt(DACH)(VPA)₂(ox)] | 0.37 ± 0.08 |
| [Pt(DACH)(VPA)(OH)(ox)] | 0.10 ± 0.05 |
| ctc-[Pt(NH₃)₂(PhB)₂Cl₂] | −0.73 ± 0.05 |
| [Pt(DACH)(PhB)₂(ox)] | 0.25 ± 0.02 |

Interestingly, there was no correlation between lipophilicity and cellular association. The highest accumulation was observed for ctc-[Pt(NH₃)₂(PhB)₂Cl₂] followed by ctc-[Pt(NH₃)₂(VPA)₂Cl₂]. Although ctc-[Pt(NH₃)₂(VPA)₂Cl₂] and [Pt(DACH)(PhB)₂(ox)] have the same log P values the former has much higher accumulation than the latter and [Pt(DACH)(VPA)₂(ox)] with the highest log P (0.37) does not accumulate as well as ctc-[Pt(NH₃)₂(PhB)₂Cl₂] and ctc-[Pt(NH₃)₂(VPA)₂Cl₂].

A very different picture unfolds when the cells (MCF-7 and A375) are incubated for 24 h with the IC₅₀ concentrations for each complex (FIG. 2). With the exception of compound IV, all the compounds show very similar levels of cellular accumulation in MCF-7 cells and with the A375. These results seem to indicate that regardless of the structure of the pro-drug when a certain amount of the complexes accumulates in the cells (˜300 ng/10⁶ cells for MCF-7 or 500 ng/10⁶ cells for A375) it leads to the death of 50% of the cancer cells.

HDAC Inhibition

The complexes described here were specifically designed to release either VPA of PhB inside the cells in the hope that they will inhibit HDAC activity paving the way for more efficient DNA platination. Thus, in addition to measuring the levels of DNA platination, we also measured the ability of these pro-drugs to inhibit HDAC activity in cells. MCF-7 cells were incubated for 24 h with IC₅₀ concentrations of compounds I-VIII and their HDAC activity was determined in cells (FIG. 3A). For comparison purposes, the HDAC inhibitory effect determined by tested complexes was also assayed in isolated nuclei of MCF-7 treated cells (FIG. 3B).

MCF-7 cells were incubated for 24 h with IC₂₅ or IC₅₀ concentrations of tested complexes or TSA (trichostatin A, HDAC inhibitor). The HDAC activity was determined by the Fluor de Lys® Fluorescent Cellular Activity Assay (Enzo Life Sciences).

In both experiments, the pattern of HDAC inhibition was very similar; resembling those obtained from cytotoxicity and platination assays. As expected, cisplatin, oxaliplatin as well as Pt(IV) complexes IX-XII lacking the HDAC inhibitors in the axial position were not effective in modifying HDAC activity. Compounds I and VI are significantly more potent HDAC inhibitors than the other derivatives. More importantly, their IC₅₀ values (μM) calculated in term of cellular HDCA inhibitory activity were three orders of magnitude lower than those reported for free VPA or PhB, respectively. As a general consideration, the bis-HDACi compounds were more efficient than the corresponding mono-HDACi. However, all the complexes were significantly less potent HDAC inhibitors compared with Trichostatin A (TSA).

Also, MCF-7 cells were incubated for 24 h with increasing concentrations of compounds I-VIII and their HDAC activity was determined. The IC₅₀ values for the inhibition of HDAC activity in the cells are depicted in FIG. 3C.

The IC₅₀ values for cellular inhibition of HDAC activity for all the compounds that release either VPA (I-V) or PhB (VI-VIII) in the cell are in the low μM range, similar to the cytotoxicity IC₅₀ values, while those for valproate (˜8 mM) and PhB are significantly higher.

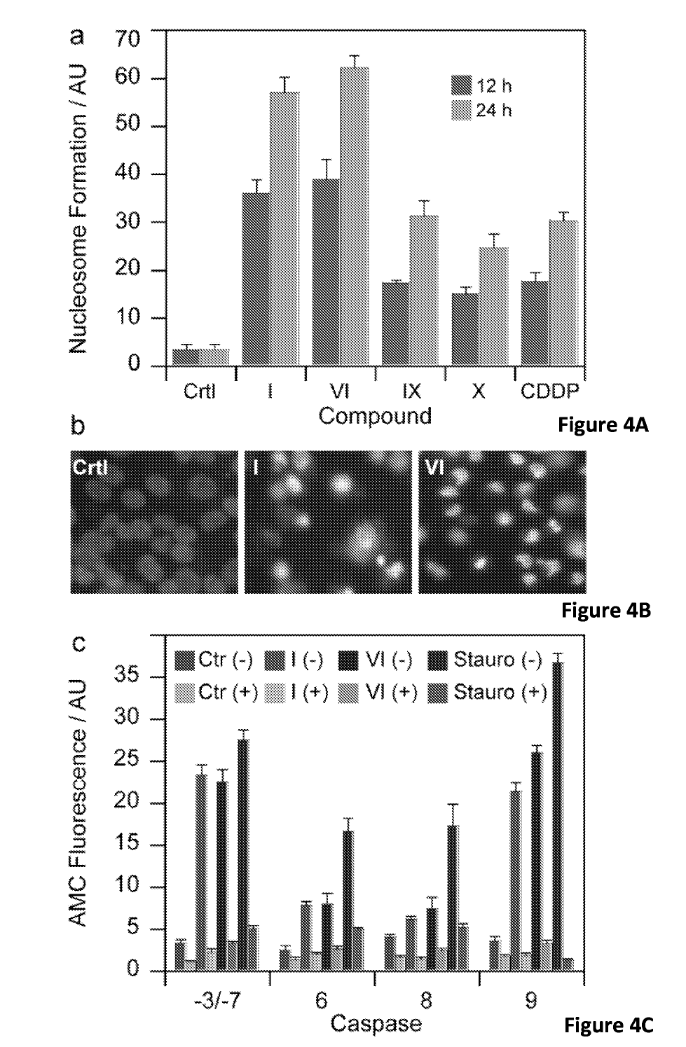

To gain more insights into the mode of action of newly synthesized Pt(IV) derivatives, compounds 1 and 6 were chosen for further experiments. In particular, their capacity to induce apoptosis against human breast MCF-7 cells treated with equitoxic concentrations (IC₅₀ values) of these drugs was evaluated and compared. Nuclear DNA fragmentation and apoptosome complex formation are critical steps in the apoptotic process. DNA fragmentation tests performed on MCF-7 cells treated for 12 or 24 h with IC₅₀ concentrations showed the ability of both Pt(IV) derivatives to increase mono- and oligo-nucleosome formation to a very similar extent in a time-dependent manner (FIG. 4A). Notably, after a 24 h treatment, 1 and 6 induced respectively a 1.5 and 2 times higher induction of nucleosome formation compared with cisplatin. Apoptotic cell death induction was confirmed through a Hoechst 33342 staining. As reported in FIG. 4B, MCF-7 cells treated with IC₅₀ concentrations of 1 and 6 displayed, chromatin condensation and fragmentation characteristics, typical of apoptosis.

To assess the mechanism of the triggered apoptotic process, the activity of the two initiator caspases (-8 and -9), and the downstream effectors (-3/-7 and -6) was determined in MCF-7 treated cultures. As shown in FIG. 4C, both derivatives provoked a substantial activation of all caspases. Notably, after 24 h of incubation with IC₅₀ concentrations of 1 or 6, cleavage of caspase-3/-7 and 9 were higher with respect to activation of caspase 6 and 8. Caspase-3/-7 and 9 cleavage reached values similar to that exerted by staurosporin, a well-known caspase-dependent apoptosis inducer.

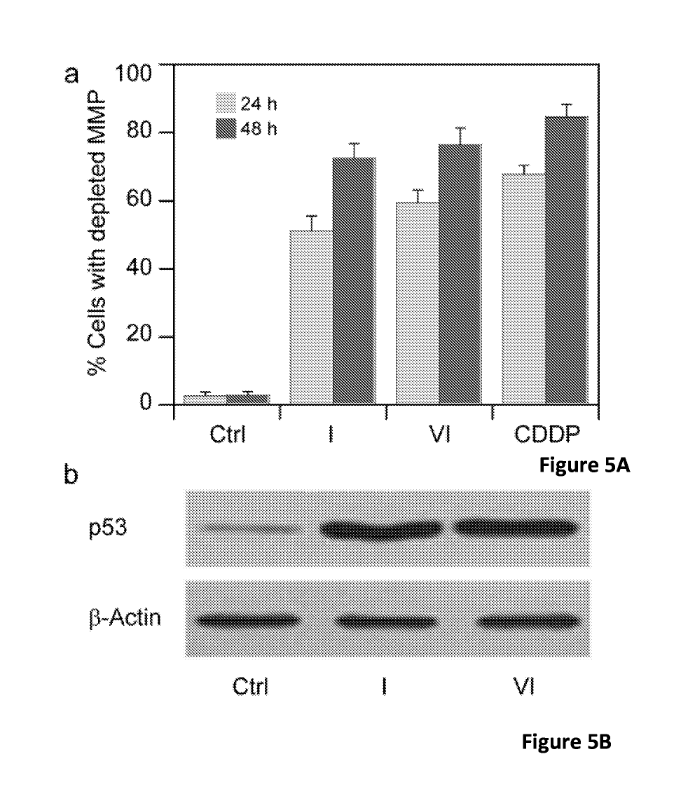

Interestingly, treatment with zVAD, a cell-permeable pan-caspase inhibitor, strongly decrease caspase activation, thus confirming the role played by these proteases in cell death determined by the novel Pt(IV) complexes. Since caspases 3/-7 and 9 are mainly involved in intrinsic apoptosis pathway, whereas caspase 6 and 8 are extrinsic caspase executors, these data attest for 1 and 6 a predominant induction of apoptotic signaling via the mitochondrial pathway. Based on these findings, we thought it would be interesting to evaluate the effect induced by Pt(IV) derivatives on mitochondria membrane potential (ΔΨ, MMP). MMP depletion generally precedes mitochondrial-driven apoptotic cell death. By treating MCF-7 cells with IC₅₀ concentrations of 1 and 6 for 12 and 24 h, JC-10 fluorescence intensity decrease by increasing exposure times (FIG. 5), thus attesting a continuous increase in cells with depleted MMP.

Satraplatin, ctc-[Pt(NH₃)(c-hexylamine)(OAc)₂Cl₂], and other Pt(VI) derivatives have been recently shown to induce cell death in human cancer cells via p53 and p21 induction. Accordingly, 24 h-treatment of MCF-7 cells with 1 and 6 resulted in an increase in p53 activation. However, by taking into account cytotoxicity profiles of 1 and 6, rather similar cytotoxicities were observed against either p53 wild-type MCF-7 cells and p53 null PC3 cell line. These data indicate that the p53 status barely influenced the sensitivity of the cancer cells to the studied compounds, thus indicating that these complexes can activate apoptotic cell death pathways by mechanisms that are both dependent and independent of p53, and can probably recruit more than one pathway to trigger cell death.

In Vivo Studies on Compound VI ctc-[Pt(NH₃)₂(PhB)₂Cl₂]

A preliminary in vivo efficacy study was carried out on a mouse model (see Table 3 below):

TABLE 3
In vivo efficacy study.

| | Daily dose | Average tumor weight | Inhibition of |
|---|---|---|---|
| | (mg kg⁻¹) | (mean ± SD, g) | tumor growth (%) |
| Control ^{a} | — | 0.563 ± 0.14 | — |
| I | 20 | 0.179 ± 0.07 | 68.21 |
| IV | 20 | 0.095 ± 0.04 | 83.13 |
| CDDP | 1.5 | 0.150 ± 0.07 | 73.35 |
| CDDP | 3 | 0.044 ± 0.07 | 92.18 |

^{a} vehicle (0.5% (v/v) of DMSO, 99.5% (v/v) of 0.9% NaCl).

Lewis lung carcinoma (LLC) was implanted i.m. (2·10⁶ cells inoculum) into the right hind leg of 8-week old inbred C57BL mice (24±3 g body weight). Chemotherapy was delayed until the tumor became visible (day 7). Day 7-14: animal received daily 20 mg/kg of I or IV orally as well as daily 1.5 and 3 mg/kg of cisplatin i.p. At day 15 animals were sacrificed, legs amputated at the proximal end of the femur, and the inhibition of tumor growth was determined as the difference in weight of the tumor-bearing leg and the healthy leg expressed as % referred to the control animals.

This preliminary results show that compound VI has curative potential by oral administration and is nearly as effective in this model as cisplatin that was administered ip.

### Discussion

The hypothesis underlying this work was that by combining agents that can increase the accessibility of nuclear DNA with DNA damaging agents that lead to the death of the cancer cells we might be able to increase the efficiency of killing cancer cells. Towards this end we designed Pt(IV) derivatives of cisplatin and oxaliplatin that have one or two HDACis as axial ligands in the hope that the cellular accumulation of these complexes will be high due to the lipophilicity of the HDAC is and once inside the cell these pro-drugs will be activated by reduction simultaneously releasing the Pt drug and the HDACi. To check this hypothesis we synthesized eight such complexes and compared their cytotoxicity to the parent Pt drugs (cisplatin or oxaliplatin) and to the HDACis (VPA of PhB).

The compounds were screened against nine different cancer cells and looking at the average IC₅₀ values of all cell lines we see that oxaliplatin is about two fold more potent than cisplatin (4.9 vs 9.9 μM). Therefore, it is worth examining whether the Pt(IV) derivatives of oxaliplatin with the same axial ligands are be more potent than their cisplatin analogs. This is clearly not the case as we see that ctc-[Pt(NH₃)₂(VPA)₂Cl₂] is significantly more potent than [Pt(DACH)(VPA)₂(ox)] with average IC₅₀s of 1.15 and 4.48 μM respectively. Ctc-[Pt(NH₃)₂(PhB)₂Cl₂] and [Pt(DACH)(PhB)₂(ox)] behave in a similar manner where their IC₅₀s are 0.23 and 6.81 μM respectively. Interestingly, attaching the two bioactive VPA ligands to the axial positions of the Pt(IV) derivative of cisplatin increased its average potency by 8 fold compared with cisplatin while doing the same for the oxaliplatin derivatives resulted in a twofold reduction in potency relative to oxaliplatin. Ctc-[Pt(NH₃)₂(PhB)₂Cl₂] is 50 times more potent than cisplatin while [Pt(DACH)(PhB)₂(ox)] is slightly less potent that oxaliplatin. Clearly, conjugation of either two VPA or two PhB to the axial positions of cisplatin significantly enhance their potency relative to cisplatin and especially when compared with free VPA or PhB whose IC₅₀ values are in the mM range.

An essential step in the cascade of events leading to the death of the cancer cells by these compounds is the accumulation in the cells. Often higher cellular accumulation correlates with improved cytotoxicty. Comparing the cellular association of the complexes following 24 h incubation to equi-Pt concentration (5 μM) showed a correlation between the cell-associated Pt levels and the IC₅₀ values. The most potent compounds, I and VI, also displayed the highest levels of cellular accumulation while IV, the least potent complex, had the lowest levels of cellular associated Pt.

The same general correlation patterns were observed for Pt accumulation in the nucleus and platination of nuclear DNA and potency. The most potent compounds (I and VI) were also the most abundant in the nucleus and displayed the highest levels of DNA platination. It seems like there is a correlation between the Pt levels associated with whole cells and their levels in the nucleus and bound to DNA. Interestingly, when the cells were exposed for 24 h to IC₅₀ values of each of the compounds the Pt levels associated with the cells for all the compounds (with the exception of IV) were very similar. This result seems to suggest that regardless of the structure of the Pt complex, in order to kill 50% of the cells, a certain number of molecules have to accumulate in the cell. In the case of MCF-7 cells approximately 350 pg Pt/10⁶ cells are required (1×10⁶ molec/cell) while for A375 cells approximately 500 pg Pt/10⁶ cells are required (1.5×10⁶ molec/cell). However, the extracellular Pt concentrations necessary to attain these conditions are different.

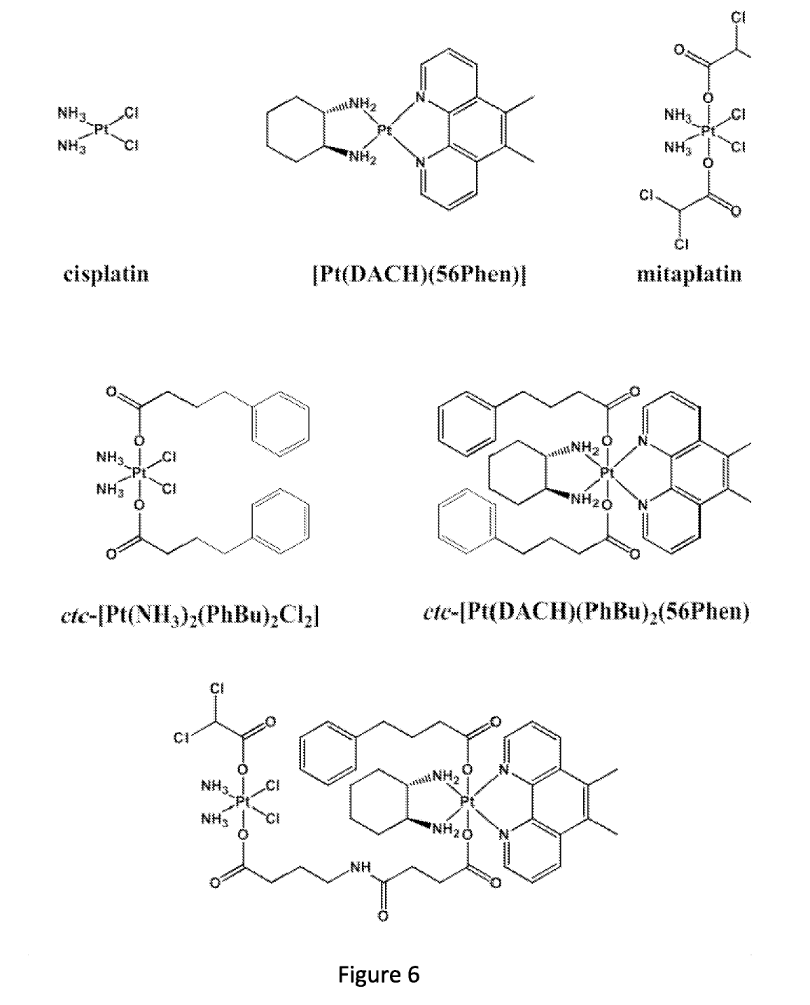

To check the validity of the working hypotheses that these complexes will inhibit HDAC activity in the cells, the ability of each of the complexes to inhibit HDAC activity in MCF-7 cells was evaluated. Cells were exposed to increasing concentration of the compounds and the HDAC activity was measured and the concentrations of the complexes that caused 50% inhibition of HDAC activity are depicted in FIG. 6. A correlation was observed between the ability to inhibit HDAC activity (IC₅₀ values for HDAC inhibition) and the cytotoxicity IC₅₀ values obtained. Again, compounds I and VI showed the highest inhibitory effect while compound IV was least effective. Notably, μM concentration of the complexes inhibit 50% HDAC activity. Following a 24 h incubation with IC₅₀ concentrations of all the compounds we see that TSA (a potent HDACi) is the most potent HDACi inhibiting about 90% of the activity of the enzymes. Compounds VI and I are more potent than the rest of the Pt complexes inhibiting approximately 70% and 65% of the HDAC activity respectively. All the other compounds also show HDAC inhibitory behavior of approximately 40-50%.

One of the interesting questions that arises is whether the high potency of compounds I and VI is due primarily to a synergistic effect between the HDAC inhibitors and the Pt complexes or is it merely due to the enhances cellular accumulation. It is generally accepted that lipophilic ligands enhance the cellular uptake of Pt complexes and that higher accumulation usually results in enhanced cytotoxicity. Thus attaching the lipophilic VPA or PhB ligands to the Pt pro-drug is expected to enhance its potency by virtue of higher cellular accumulation. The situation is quite different for the HDACis used in this study. Both valproic acid and 4-phenylbutyruc acid are ionized at neutral pH and are monoanionic and therefore will not efficiently accumulate in the cancer cells. The results show that valproate does not accumulate in the cells very efficiently. Attempts to increase the cellular accumulation of valproate include neutralization of the charge by esterifying the carboxylate and forming for instance valproyl ester-valpramide of acyclovir. Upon ligating the VPA or PhB to the axial positions of the Pt(IV) complex a metalloester is formed and the charges of the VPA and PhB are neutralized facilitating the cellular uptake. Thus, conjugation of VPA or PhB to the Pt(IV) yields complexes that facilitate the cellular uptake of both its components.

Higher intracellular concentrations of both VPA/PhB and the cytotoxic Pt(II) drugs are likely to be more effective at inducing apoptosis. Although VPA/PhB were selected because they have the ability to inhibit HDAC activity, but the activity of HDAC enzymes is not limited to histones and they can modify other proteins such as chaperones, transcription regulators, transcription factors, signal transduction mediators and others. VPA can deplete GSH levels in rat cells.

The growth inhibitory effect of PBA on gastric cancer cells is associated with alteration of the cell cycle. In another publication, phenyl butyric acid is reported to rescue endoplasmic reticulum stress-induced suppression of APP proteolysis and prevents apoptosis in neuronal cells. These examples demonstrate that efficient importation of VPA or PhB into cell by the Pt(IV) pro-drugs can lead to many different cellular outcomes, including HDAC inhibition and suppression of expressions of HDAC enzymes, that could in conjunction with cisplatin enhance the efficiency of killing the cancer cells. It is not clear which of the cellular events, or combination of event triggered by the HDAC is contributes to the enhances potency in conjunction with the Pt moiety.

As indicated herein, the present invention also contemplates quadruple action Pt(IV) dinuclear compounds that incorporate in one molecular entity cisplatin, [Pt(DACH)(56Phen)], phenylbutyrate and dichloroacetate. This compound, after intracellular reduction, can aim DNA, histones and mitochondria as cellular targets.

### Experimental Part

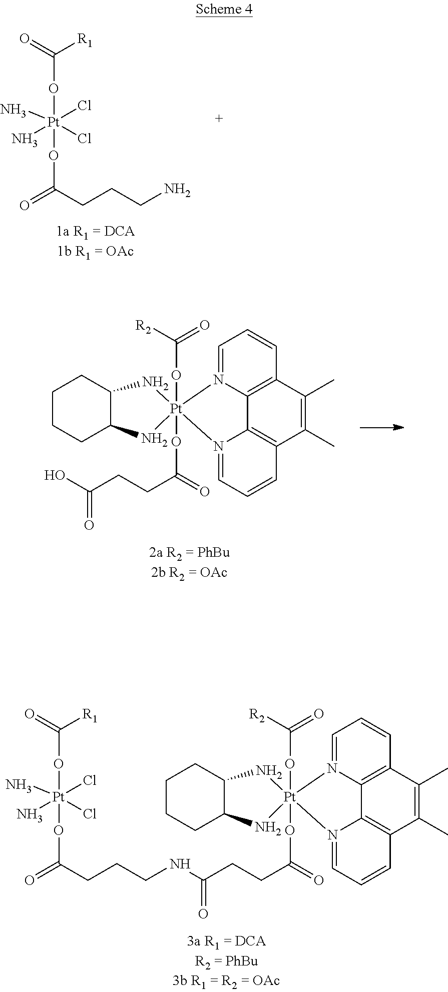

Commercial reagents and solvents were used as received, without further purification.

c,t,c-[Pt(DACH)(OH)₂(56Phen)]Cl₂, (PhBu)₂O were synthesized according procedures previously reported in the literature.

Reaction mixtures and purified products were analyzed on a Thermo Scientific UltimaMate 3000 station, equipped with a reverse-phase C18 column (Phenomenex Kinetex 250×4.6 mm, 5 μm, 100 Å). UV detection was set at 220 and 260 nm. The samples were eluted with a 0-90% linear gradient of acetonitrile in water with 0.1% trifluoroacetic acid (TFA) over 30 min.

Reaction mixtures were separated on a Thermo Scientific UltimaMate 3000 station, equipped with a reverse-phase C18 column (Phenomenex Luna 250 X 21.2 mm, 10 μm, 100 Å). UV detection was set at 220 nm. The samples were eluted with a 0-90% linear gradient of acetonitrile in water with 0.1% trifluoroacetic acid (TFA) over 30 min.

c,c,t-[Pt(NH₃)₂Cl₂(DCA)(GABA)](TFA) (1a, Scheme 4).

c,c,t-[Pt(NH₃)₂Cl₂(OH)₂] (96.2 mg, 0.29 mmol) was suspended in 10 mL of DMSO. GABA-BOC anhydride (123.0 mg, 0.32 mmol) was added and it was left to react overnight at RT. The mixture turned to a yellow solution and was centrifuged. The mother liquor was treated with ether, obtaining a two phases system that was centrifuged in order to remove the ether phase. This process was repeated several times until a sticky yellow solid was obtained. It was dissolved in methanol and precipitated with ether, affording a pale yellow solid. It was recovered by centrifugation, dried and dissolved in 3 mL of DMF. DCA anhydride (265 μL, 1.74 mmol) was added to the mixture. After 4 h at RT, the mixture had turned to a bright yellow solution. It was filtered and then evaporated to dryness, affording a yellow sticky solid. It was dissolved in methanol and precipitated with ether. The solid was recovered by centrifugation and dried. It was stirred at room temperature in a mix of DCM and TFA (1:1) for 45 min. The solvents were removed with a stream of compressed air. A sticky yellow solid was obtained. It was dissolved in methanol and added I a dropwise fashion to ice-cold ether, inducing the formation of a white precipitate. The solid was collected by centrifugation and dried under a stream of compressed air. 52.0 mg were recovered (28% yield). This complex was used for the synthesis of 3a (Scheme 4) without further purification. It was purified on preparative HPLC (retention time=11.8 min) and lyophilized, when used for biological studies. ¹H NMR δ (D₂O): 6.24 (s, 1H), 2.96 (t, 2H), 2.48 (t, 2H), 1.82 (m, 2H). ¹H NMR δ (D₂O): 1076 ppm. Retention time on analytical HPLC: 8.4 min ESI-MS: calculated for [M+H]⁺ (M=C₆H₁₅ Cl₄N₃O₄Pt): m/z=531.10; experimental: m/z=531.92.

c,c,t-[Pt(NH₃)₂Cl₂(OAc)(GABA)](TFA) (1b, Scheme 4).

c,c,t-[Pt(NH₃)₂Cl₂(OAc)(OH)] (113.3 mg, 0.30 mmol) was suspended in 5 mL of DMF. GABA-BOC anhydride (234.0 mg, 0.60 mmol) was added and then the mixture was stirred overnight at 40° C., affording a yellow solution that was filtered and then reduced to dryness under reduce pressure. The sticky yellow solid was then dissolved in methanol and precipitated with ether. The precipitate was collected by centrifugation and dried. It was then stirred at room temperature in a mix of DCM and TFA (1:1) for 45 min. The solvents were removed with a stream of compressed air. The sticky solid was dissolved in methanol and added dropwise to ice-cold ether, inducing the formation of a white precipitate. The solid was collected by centrifugation and dried under a stream of compressed air. 59.2 mg were recovered (34% yield). This complex was used for the synthesis of 3b (Scheme 4) without further purification. ¹H NMR δ (D₂O): 2.94 (t, 2H), 2.44 (t, 2H), 2.00 (s, 3H), 1.81 (q, 2H). ¹H NMR δ (D₂O): 1077 ppm. Retention time on analytical HPLC: 3.4 min ESI-MS: calculated for [M+H]⁺ (M=C₆H₁₇Cl₂N₃O₄Pt): m/z=462.21; experimental: m/z=462.14.

c,c,t-[Pt(DACH)(56Phen)(PhBu)(Succ)]Cl₂ (2a, Scheme 4).

c,c,t-[Pt(DACH)(56Phen)(OH)₂]Cl₂ (111.5 mg, 0.18 mmol) was stirred in 4 mL of DMSO together with phenylbutyric anhydride (67.0 mg, 0.22 mmol) at RT for 2 h. Afterwards, succinic anhydride (72.0 mg, 0.72 mmol) was added to the mixture. After one night, the reaction mixture was filtered. The filtrate was diluted with EtOAc, and then precipitated with ether. The brown sticky precipitate was recovered by centrifugation, dried and then dissolved in methanol and precipitated with ether, affording a beige precipitate that was recovered by centrifugation and then dried. 92.0 mg were recovered (60% yield). This complex was used for the synthesis of 3a (Scheme 4) without further purification. It was purified on preparative HPLC (retention time=15.6 min), obtaining the TFA salt c,t,c-[Pt(DACH)(PhBu)(Succ)(56Phen)](TFA), and lyophilized, when used for biological studies. ¹H NMR δ (D₂O): 9.38 (d, 2H), 9.18 (m, 2H), 8.29 (m, 2H), 7.16 (m, 3H), 6.47 (m, 2H), 3.30 (d, 1H), 3.20 (d, 1H), 2.71 (s, 6H), 2.46 (m, 2H), 2.32 (t, 2H), 2.14 (t, 2H), 2.08 (M, 2H), 1.80 (m, 2H), 1.74 (M, 2h), 1.1 (M, 4H), 1.18 (t, 2H). ¹⁰⁵Pt NMR δ (D₂O): 700 ppm. Retention time on analytical HPLC: 14.6 min. ESI-MS: calculated for [M−H]⁺ (M=C₃₄H₄₂N₄O₆Pt): m/z=796.27; experimental: m/z=795.94.

c,c,t-[Pt(DACH)(56Phen)(OAc)(Succ)]Cl₂ (2b, Scheme 4).

c,c,t-[Pt(DACH)(56Phen)(OH)₂]Cl₂ (337.8 mg, 0.54 mmol) was suspended in 12 mL of DMSO. Succinic anhydride (54.3 mg, 0.54 mmol) was added and the mixture was stirred overnight at room temperature in the dark. The next day, an excess of acetic anhydride (6 mL) was added. After 4 h, the mixture was filtered, diluted with EtOAc and precipitated with ether. The brown sticky precipitate was collected by centrifugation, dried and then dissolved in methanol and reprecipitated with ether. The beige precipitate was collected by centrifugation and dried. 614.8 mg were recovered (80% yield). This complex was used for the following reactions without further purification. This complex was used for the synthesis of 3b (Scheme 4) without further purification. ¹H NMR δ (D₂O): 9.14 (d, 2H), 9.07 (d, 2H), 8.09 (t, 2H), 3.07 (d, 2H), 2.73 (s, 6H). 2.29 (d, 2H), 2.10 (m, 2H), 1.99 (m, 2H), 1.61 (b, 2H), 1.60 (s, 3H), 1.56 (b, 2H), 1.20 (b, 2H). ¹H NMR δ (D₂O): 702 ppm. Retention time on analytical HPLC: 9.5 min ESI-MS: calculated for [M−H]⁺ (M=C₂₆H₃₄N₄O₆Pt): m/z=692.20; experimental: m/z=692.18.

c,c,t,t,c,c-[(DACH)(56Phen)(PhBu)Pt(μ-OOC—(CH₂)₂—CONH(CH₂)₃COO)—Pt(DCA)(NH₃)₂ (Cl₂)](TFA)₂ (3a, Scheme 1).

2a (86.8 mg, 0.10 mmol) was stirred in 1.0 mL of DMSO. EDC (38.3 mg, 0.20 mmol) and NHS (23.0 mg, 0.20 μmol) were added to the mixture, and it was stirred at room temperature in the dark for 45 minutes. 1a (64.9 mg, 0.10 mmol) was dissolved in 1.0 mL of DMSO in the presence of TEA (28.0 μl, 0.20 mmol) and added to the mixture. The mixture was kept under magnetic stirring at RT for 4 h. Afterwards, the mixture was diluted with EtOAc and precipitated with ether, affording a sticky dark brown precipitate. It was recovered by centrifugation, dried, dissolved in methanol and precipitated with ether. The resulting beige precipitate was collected by centrifugation and dried. Afterwards, it was dissolved in a mixture of water and ACN (1:1), purified on preparative HPLC (retention time=17.6 min), and finally lyophilized. 30.0 mg were recovered (20% yield). ¹H NMR δ (D₂O): 9.41 (d, 2H), 9.22 (m, 2H), 8.34 (m, 2H), 7.07 (d, 2H), 7.06 (m, 1H), 6.36 (m, 2H), 6.25 (s, 1H), 3.31 (m, 1H), 3.23 (m, 1H), 2.73 (s, 3H), 2.71 (t, 2H), 2.48 (m, 2H), 2.44 (m, 2H), 2.25 (m, 2H), 2.13 (m, 2H), 2.09 (m, 2H), 1.77 (m, 2H), 1.47 (m, 2H), 1.38 (m, 4H), 1.32 (m, 2H), 1.17 ppm (m, 2H). ¹H NMR δ (D₂O): 1082 and 701 ppm. Retention time on analytical HPLC: 16.6 min ESI-MS: calculated for [M−H]⁺ (M=C₄₀H₅₅ Cl₄N₇O₉Pt₂): m/z=1308.54; experimental: m/z=1308.87.

c,c,t,t,c,c-[(DACH)(56Phen)(OAc)Pt(μ-OOC—(CH₂)₂—CONH(CH₂)₃COO)-Pt(OAc)(NH₃)₂(Cl₂)](TFA)₂ (3b, Scheme 4).

2b (104.3 mg, 0.14 mmol) was stirred in 1.2 mL of DMSO. EDC (53.7 mg, 0.28 mmol) and NHS (32.2 mg, 0.28 mmol) were added and the mixture was stirred at RT for 45 min. Afterwards, 1b (78.5 mg, 0.14 mmol), previously dissolved in 1.2 mL of DMSO in the presence of TEA (39.0 μl, 0.28 mmol), was added to the mixture. The mixture was kept under magnetic stirring at RT for 4 h. Afterwards, the mixture was diluted with EtOAc and precipitated with ether, affording a sticky dark brown precipitate. It was recovered by centrifugation, dried, dissolved in methanol and precipitated with ether. The resulting beige precipitate was collected by centrifugation and dried. Afterwards, it was dissolved in a mixture of water and ACN (1:1), purified on preparative HPLC (retention time=11.6 min), and finally lyophilized. 20.0 mg were recovered (10% yield). ¹H NMR δ (D₂O): 9.21 (d, 2H), 9.14 (d, 2H), 8.17 (t, 2H), 3.12 (d, 2H), 2.79 (s, 6H, CH₃), 2.55 (t, 2H), 2.35 (d, 2H), 2.22 (m, 2H), 2.14 (t, 2H), 2.01 (s, 3H), 1.96 (m, 2H), 1.66 (b, 2H), 1.64 (s, 3H), 1.63 (b, 2H), 1.35 (q, 2H), 1.26 (b, 2H) ppm. ¹H NMR δ (D₂O): 1081 and 707 ppm. Retention time on analytical HPLC: 10.0 min. ESI-MS: calculated for [M−H]⁺ (M=C₃₂H₄₉Cl₂N₇O₉Pt₂): m/z=1134.22; experimental: m/z=1134.86.

Study of Stability in Buffer.

A solution 2.0 mM of 3a (Scheme 4) in sodium phosphate buffer (50 mM, pH=7) was kept at 37° C. for 3 days. NMR and HPLC measurements were performed every day.

Study of Stability in Cell Culture Medium.

A solution 2.0 mM of 3a (Scheme 4) in DMEM was kept at 37° C. for 3 days. NMR measurements were performed every day.

Study of Reduction in Buffer in the Presence of Ascorbic Acid.

A solution 2.0 mM of 3a (Scheme 4) in buffer was treated with 1 Eq ascorbic acid. NMR measurements were performed over a 2 hour time frame.

Cytotoxicity

MTT assay. The growth inhibitory effect towards human cell lines was evaluated by means of MTT (tetrazolium salt reduction) assay. Briefly, 3-8.10³ cells/well, dependent upon the growth characteristics of the cell line, were seeded in 96-well microplates in growth medium (100 μL) and then incubated at 37° C. in a 5% carbon dioxide atmosphere. After 24 h, the medium was removed and replaced with a fresh one containing the compound to be studied at the appropriate concentration. Triplicate cultures were established for each treatment. After 72 h, each well was treated with 10 μL of a 5 mg·mL−1 MTT (3-(4,5-dimethylthiazol-2-yl)-2,5-diphenyltetrazolium bromide) saline solution, and after 5 h additional incubation, 100 μL of a sodium dodecylsulfate (SDS) solution in HCl 0.01 M were added. After overnight incubation, the inhibition of cell growth induced by the tested complexes was detected by measuring the absorbance of each well at 570 nm using a Bio-Rad 680 microplate reader (Bio-Rad, Hercules, Calif.). Mean absorbance for each drug dose was expressed as a percentage of the control untreated well absorbance and plotted vs drug concentration. IC₅₀ values represent the drug concentrations that reduced the mean absorbance at 570 nm to 50% of those in the untreated control wells.

Acid phosphatase (APH) assay. A modified APH assay, which is based on quantification of cytosolic acid phosphatase activity, was used for determining cell viability in spheroids. Briefly, the pre-seeded spheroids were treated with fresh medium containing the compound to be studied at the appropriate concentration. Triplicate cultures were established for each treatment. After 72 h, each well was treated with 100 μL of the assay buffer (0.1 M sodium acetate, 0.1% Triton-X-100, supplemented with ImmunoPure p-nitrophenyl phosphate; Sigma Chemical Co.) and, following 3 h of incubation, 10 μL of 1 M NaOH solution were added. The inhibition of the cell growth induced by the tested complexes was detected by measuring the absorbance of each well at 405 nm, using a Bio-Rad 680 microplate reader. Mean absorbance for each drug dose was expressed as a percentage of the control untreated well absorbance (T/C) and plotted vs drug concentration. IC₅₀ values, the drug concentrations that reduce the mean absorbance at 405 nm 50% of those in the untreated control wells, were calculated by four parameter logistic (4-PL) model. Evaluation was based on means from at least four independent experiments.

Cellular accumulation and distribution. 2008 and PSN1 cells (2.5.10⁶) were seeded in 75 cm² flasks in growth medium (20 mL). After overnight incubation, the medium was replaced and the cells were treated with tested compounds for 24 h. Cell monolayers were washed twice with cold PBS, harvested and counted. Cell nuclei were isolated by means of the nuclei isolation kit Nuclei EZ Prep (Sigma Co.) as well as cellular mitochondrial fractions were isolated by Mitochondria Isolation Kit (Sigma Co.). Samples were than subjected to three freezing/thawing cycles at −80° C., and then vigorously vortexed. The samples were treated with highly pure nitric acid (Pt: ≤0.01 μg·kg⁻¹, TraceSELECT® Ultra, Sigma Chemical Co.) and transferred into a microwave teflon vessel. Subsequently, samples were submitted to standard procedures using a speed wave MWS-3 Berghof instrument (Eningen, Germany) After cooling, each mineralized sample was analyzed for platinum by using a Varian AA Duo graphite furnace atomic absorption spectrometer (Varian, Palo Alto, Calif.; USA) at the wavelength of 324.7 nm. The calibration curve was obtained using known concentrations of standard solutions purchased from Sigma Chemical Co. purchased from Sigma Chemical Co.

DNA platination. 2008 and PSN1 cells (3.10⁶) were seeded in 10 cm Petri dishes in 10 mL of culture medium. Subsequently, cells were treated with tested complexes for 24 h. DNA was extracted and purified by a commercial spin column quantification kit (Qiagen DNeasy Blood and Tissue Kit). Only highly purified samples (A260/A230 □ 1.8 and A280/A260 □ 2.0) were included for analysis to avoid any artifacts. The samples were completely dried and re-dissolved in 200 μL of Milli-Q water (18.2 MΩ) for at least 20 min at 65° C. in a shaking thermo-mixer, mineralized and analyzed for total Pt content by GF-AAS as described above.

Histone deacetylase assay. Histone deacetylase activity was determined using Fluor-de-Lys® HDAC fluorometric activity assay kit (Enzo Life Sciences International, Inc., Plymount Meeting, Pa., U.S.A.). 2008 and PSN1 cells (5.10⁴ seeded in 96-well microplates) were treated for 24 h with tested complexes, and then processed as reported by the manufacturer's instructions. Fluorescence was measured using a Fluoroskan Ascent FL (Labsystem, Finland) plate reader, with excitation at 360 nm and emission at 460 nm.

Mitochondrial membrane potential (ΔΨ). ΔΨ was assayed using the Mito-ID Membrane Potential Kit according to the manufacturer's instructions (Enzo Life Sciences, Farmingdale, N.Y.). Briefly, 2008 and PSN1 cells were seeded onto 96-well microplates at 5.10⁴ cells/well. After 24 h, cells were treated with tested compounds for 6, 12 and 24 h. The mitochondrial depolarizing agent, carbonyl cyanide 3-chlorophenylhydrazone (CCCP) was employed as positive control. An equal volume of cationic dye loading solution was added to each well and cell plates were incubated for additional 30 min at 37° C. Plates were read at excitation/emission wavelengths 490 and 590 nm using a fluorescence microplate reader (Fluoroskan Ascent FL, Labsystem, Finland).

Glycolysis. The cellular glycolytic activity was assessed using the Mito-ID® O₂ Extracellular Sensor Kit according to the manufacturer's instructions (Enzo Life Sciences, Farmingdale, N.Y.). Briefly, PSN-1 cells (15·10³ per well) were seeded in 96-well plates. After 24 h, cells were treated with Mito-ID® O₂ Sensor Probe Solution containing the tested compounds at the appropriate concentration. Fluorescence was estimated using a plate reader (Fluoroskan Ascent FL, Labsystem, Finland) at 350 nm (excitation) and 610 nm (emission).

Transmission electron microscopy analyses. About 10⁶ PSN-1 cells were seeded in 24-well plates and, after 24 h incubation, were treated with the tested compounds and incubated for additional 24 h. Cells were then washed with cold PBS, harvested and directly fixed with 1.5% glutaraldehyde buffer with 0.2 M sodium cacodylate, pH 7.4. After washing with buffer and postfixation with 1% OsO₄ in 0.2 M cacodylate buffer, specimens were dehydrated and embedded in epoxy resin (Epon Araldite). Sagittal serial sections (1 μm) were counterstained with toluidine blue; thin sections (90 nm) were given contrast by staining with uranyl acetate and lead citrate. Micrographs were taken with a Hitachi H-600 electron microscope (Hitachi, Tokyo, Japan) operating at 75 kV. All photos were typeset in Corel Draw 11.

Hoechst 33258 staining. PSN-1 cells were seeded into 8-well tissue-culture slides (BD Falcon, Bedford, Mass., USA) at 5.10⁴ cells/well (0.8 cm²). After 24 h, cells were washed twice with PBS and following 48 and 72 h of treatment with IC₅₀ doses of the tested compound, cells were stained for 5 min with 10 μg/mL of Hoechst 33258 (2′-(4-hydroxyphenyl)-5-(4-methyl-1-piperazinyl)-2,5′-bi-1H-benzimidazole 3HCl hydrate, Sigma-Aldrich) in PBS before being examined by fluorescence microscopy (Olympus).

### Results and Discussion

Synthesis and Characterization

In order to obtain the quadruple action Pt(IV) dinuclear compound 3a (Scheme 4), we synthesized two separate Pt(IV) units, 1a and 2a (Scheme 4), each of them with both one bioactive and one bridging moiety in the axial positions, and finally we condensed the two units, obtaining the dinuclear complex.

In the case of the unit 1a (Scheme 4), we started from oxoplatin, ctc-[Pt(NH₃)₂(OH)₂Cl₂], the axial dihydroxido Pt(IV) derivative of cisplatin. We performed the first carboxylation reaction in DMSO using a small excess of the anhydride (1.1 Eq), under dilute conditions (ca. 10 mg of Pt complex/mL). Initially, a GABA-BOC moiety was conjugated to one of the axial positions. The initial suspension turned to a solution after one day. The monocarboxy derivative, that was identified by ¹H NMR=1050 ppm), was isolated by precipitation with ether dissolved in DMF and was then reacted with DCA anhydride. The reaction was monitored by ¹H NMR spectroscopy: the Pt resonance shifted from 1050 to 1170 ppm upon carboxylation. We chose to begin the carboxylation with the least reactive anhydride (GABA-BOC anhydride) in order to minimize biscarboxylation andoptimize the yield for the monocarboxylation. The second carboxylation proceeded smoothly with the more reactive and less hindered DCA. Finally, the protecting BOC group was removed in a mixture of DCM and TFA, leaving a free aminic function suitable for further reactions.

Unit 2a (Scheme 4) was prepared by initial reaction of Pt56MeSS(OH)₂ with a small excess (1.2 Eq) of phenylbutyric anhydride. The reaction was monitored by ¹⁹⁵Pt NMR spectroscopy: upon carboxylation the Pt resonance shifted from 349 to 462 ppm. Once the reaction was complete, an excess of the second anhydride was added to the mixture, without isolating the monocaroxylic derivative. Pt resonance shifted to 699 ppm after the reaction was complete. This second unit had a free carboxylic function on the succinic anhydride, suitable for further reactions.

Finally, units 1a and 2a (Scheme 4) were used for the formation of a peptide bond, to afford 3a. DMSO turned out to be a good solvent for this reaction, although DMF also afforded good results. ¹H NMR assignments for 1a, 2a, and 3a (Scheme 4) are summarized in Table 4. Cylic voltammetric measurements (CV) were performed on complex 3a and 3b finding Eₚ values of −0.462 and −0.058 V. The first reduction peak was assigned to the reduction of the cisplatin-derivative portion of the dinuclear complex, because of similarity with the values reported for similar complexes. The less negative reduction peak was assigned to the Pt-Phenantroline metal center, because of similarity to the values reported for the bis axial phenylbutyrato Pt(IV) complex Pt56MeSS(PhB)₂.

The synthesis compound 3b (Scheme 4), a control compound that has acetates instead of bioactive moieties in the axial positions, but acetates, was achieved via a similar pathway. Unit 1b (Scheme 4) was synthesized starting from the mono acetato Pt(IV) derivative of cisplatin, obtained by oxidation of cisplatin in acetic acid, which then reacted with GABA-BOC anhydride, to afford the biscarboxylato species, that underwent a deprotection process to free the aminic end of GABA. For the synthesis of the unit 2b (Scheme 4), the first carboxylation step was carried out with succinic anhydride, and the second with acetic anhydride. Finally, 1b and 2b (Scheme 4) were condensed to afford 3b (Scheme 4). ¹H NMR assignments for 1b, 2b, and 3b (Scheme 4) are summarized in Table 4.

TABLE 4
¹H and ¹⁹⁵Pt NMR chemical shift in D₂O (ppm).

| | | | | | | | ¹⁹⁵Pt NMR |
|---|---|---|---|---|---|---|---|
| | ¹H NMR chemical shift (ppm) | | | | | | chemical |
| Complex | R₁ | GABA | Succ | R₂ | DACH | 56Phen | shift (ppm) |
| 1a | 6.24 (CHCl₂). | 2.96 (C_{γ}H₂), | / | / | / | / | 1076 |
| | | 2.48 (C_{α}H₂), | | | | | |
| | | 1.82 (C_{β}H₂). | | | | | |
| 1b | 2.00 (CH3). | 2.94 (C_{γ}H₂), | / | / | / | / | 1075 |
| | | 2.44 (C_{α}H₂), | | | | | |
| | | 1.81 (C_{β}H₂). | | | | | |
| 2a | / | / | 2.08 (C_{β}H₂), | 7.16 (o-H and | 3.30 (C_{α}H), | 9.38 (H2), | 700 |
| | | | 1.80 (C_{α}H₂). | p-H), 6.47 (m- | 3.20 (C_{α}H), | 9.18 (H4), | |
| | | | | H), 2.32 | 2.46 (C_{β}H), | 8.29 (H3), | |
| | | | | (C_{γ}H₂), 2.14 | 1.74 (CβH), | 2.71 (CH₃). | |
| | | | | (C_{α}H₂), 1.18 | 1.31 (C_{y}H). | | |
| | | | | (C_{β}H₂). | | | |
| 2b | / | / | 2.10 (C_{β}H₂), | 1.60 (CH₃). | 3.07 (CαH), | 9.14 (H2 | 702 |
| | | | 1.99 (C_{α}H₂). | | 2.29 (CβH), | and H9), | |
| | | | | | 1.61 (CβH), | 9.07 (H4 | |
| | | | | | 1.56 (CγH), | and H7) | |
| | | | | | 1.20 (CγH). | 8.09 (H3 | |
| | | | | | | and H8), | |
| | | | | | | 2.73 | |
| | | | | | | (CH3). | |
| 3a | 6.25 (CHCl₂). | 2.71 (C_{γ}H₂), | 2.44 (C_{β}H₂), | 7.07 (o-H), | 3.31 (C_{α}H), | 9.41 (H2), | 1081, 701 |
| | | 2.25 (C_{α}H₂), | 2.13 (C_{α}H₂). | 7.06 (p-H), | 3.23 (C_{α}H), | 9.22, (H4), | |
| | | 1.47 (C_{β}H₂). | | 6.36 (m-H), | 2.48 (C_{β}H), | 8.34 (H3), | |
| | | | | 2.09 (C_{γ}H₂), | 1.77 (C_{β}H), | 2.73 | |
| | | | | 1.32 (C_{α}H₂), | 1.38 (C_{γ}H), | (CH3). | |
| | | | | 1.17 (C_{β}H₂). | | | |
| 3b | 2.01 (CH₃). | 2.55 (CγH2), | 2.22 (CβH2), | 1.64 (CH3). | 3.12 (CαH), | 9.21 (H2 | 1081, 706 |
| | | 2.14 (CαH2), | 1.96 (CαH2). | | 2.35 (CβH), | and H9), | |
| | | 1.35 (CβH2). | | | 1.66 (CβH), | 9.14 (H4 | |
| | | | | | 1.63 (CγH), | and H7), | |
| | | | | | 1.26 (CγH). | 8.17 (H3 | |
| | | | | | | and H8), | |
| | | | | | | 2.79 | |
| | | | | | | (CH3). | |

Stability in Buffer

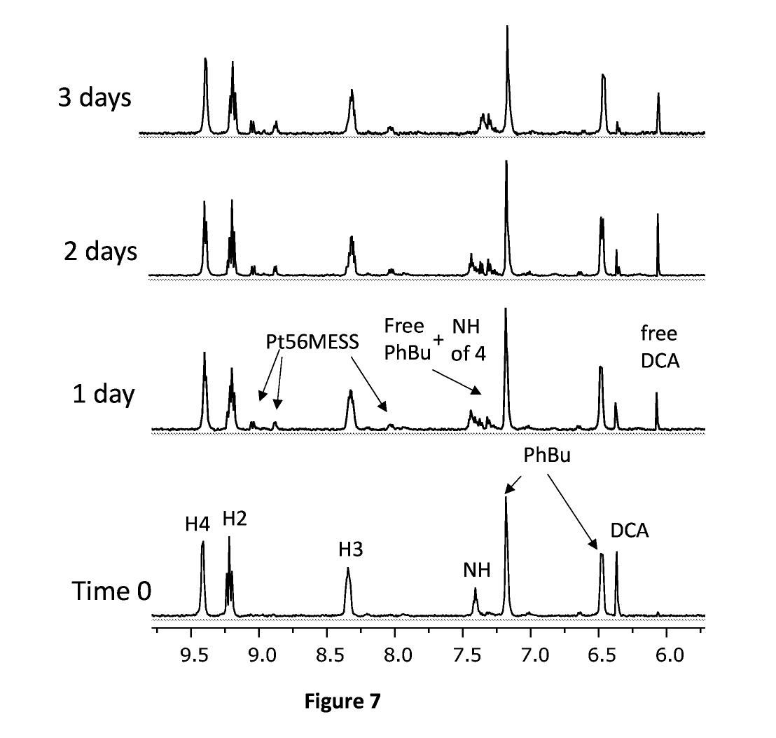

A 2.0 mM solution of 3a in phosphate buffer at pH=7 was kept at 37° C. in order to assay its stability by mean of NMR spectroscopy (FIG. 7). The ¹H NMR spectrum recorded at time 0 showed the signals of H4, H2 and H3 of the phenantroline moiety of 3a, at 9.41, 9.21 and 8.34 ppm, respectively. The signal at 7.40 ppm was assigned to the amidic proton. The signals at 7.18 and 6.48 belong to the aromatic ring of the coordinated phenylbutyrate. The large difference in chemical shifts between the 2 ortho protons of the phenyl ring and the 3 meta and para protons, coupled with the unusually low chemical shifts for the aromatic ortho protons, indicates a strong intramolecular interaction with the phenanthroline. Finally, the peak at 6.37 belongs to the coordinated DCA.

After one day, a new series of low intensity peaks was observed in the ¹H NMR. These new signals at 9.06, 8.88 and 8.02 ppm were assigned to the aromatic protons of the Pt(II) complex [Pt(DACH)(56Phen)]²⁺, indicating that some reduction of the Pt(IV) that is ligated to the phenanthroline moiety occurred. In the 7.45-7.28 ppm range, a series of new signals overlapping with the signal of the amidic proton of 3a appeared. These new signals belong to the free phenylbutyric acid, resulting from the reductive elimination. From the relative integrals of the phenanthroline protons it can be estimated that after one day approximately 6.5% of the Pt(IV)-phen was reduced, after 2 days about 9% and after 3 days 10%.

The new signal at 6.06 ppm was assigned to free DCA. The free DCA could result from hydrolysis or from reduction of the Pt(IV)-cisPt moiety. To sort this out, ¹H NMR spectra were measured at time zero and every day. Initially two peaks with nearly equal intensity were observed in the Pt(IV) region: one peak at 1101 ppm assigned to Pt(IV)-cisPt and the other at 703 ppm assigned to Pt(IV)-phen. After a day the signal at 1101 was drastically reduced and was only 15% of the signal at 703 ppm. After the second day only the signal of the Pt(IV)-phen could be observed suggesting that all the Pt(IV)-cisPt was reduced or completely hydrolysed. We looked for the potential reduction products (cisplatin and its hydrolysis products) in the Pt(II) region but did not see any signals at all.

These observations, together with the presence of a solid in the mixture, led us to hypothesize that the DCA ligand bound to the cisplatin-like metal center was rapidly hydrolyzed and was replaced by an OH releasing free DCA into solution. Then the linker chain was also hydrolyzed releasing oxoplatin, c,c,t-[Pt(NH₃)₂Cl₂(OH)₂], as a precipitate. The instability of Pt(IV) derivatives of cisplatin with DCA was previously demonstrated by us. The poorly soluble complex c,c,t-[Pt(NH₃)₂Cl₂(OH)₂], that precipitated cannot be observed in the spectrum.

This led to the formation of a new species:

c,c,t-[Pt(DACH)(phen)(PhB)(O₂CCH₂CH₂CONHCH₂CH₂CH₂CO₂H)]²⁺ (4). (Scheme 5). The HPLC of the reaction mixture at this stage showed a peak at 14.6 min. This peak was isolated and analyzed by ¹H NMR and ESI-MS experiments, confirming the hypothesized structure of 4.

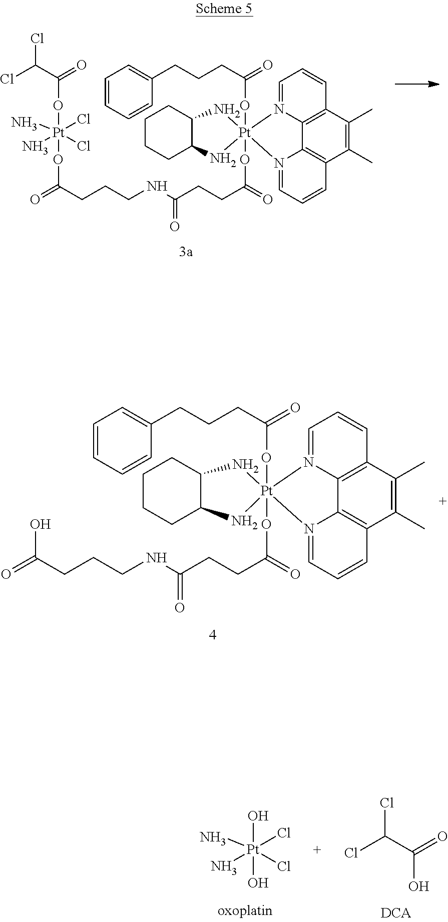

Stability in Cell Culture Medium

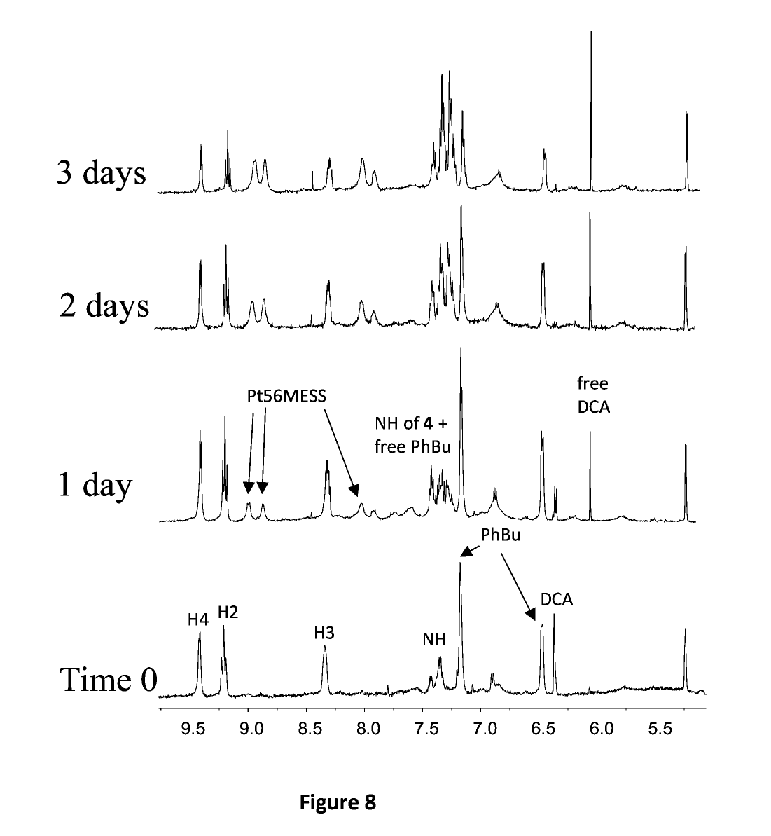

A phosphate buffer solution is an oversimplified model for medium of biological relevance in order to test the stability of a drug candidate. On one hand its simplicity allows to study degradation reactions easily, since the number of potentially interfering species is reduced to the minimum; on the other hand it does not provide a very reliable overview of what the fate of the drug candidate could be once administered. Thus, as following step, we evaluated the stability of 3a in cell culture medium DMEM, where the biomolecules present can affect the overall degradation process. A 2.0 mM solution of 3a in DMEM monitored by ¹H NMR spectroscopy for 3 days at 37° C. (FIG. 8).

Immediately after the preparation of the NMR sample, it was possible to find the signals of H4, H2 and H3 of the phenanthroline moiety of 3a, at 9.41, 9.21 and 8.34 ppm, respectively. The signal at 7.40 ppm was assigned to the amidic proton. The signals at 7.18 and 6.48 belonged to the aromatic ring of the coordinated phenylbutyrate. Finally, the peak at 6.37 belongs to the coordinated DCA. After 1 day, the signal of the reduction product [Pt(DACH)(56Phen)]²⁺ appeared at 9.00, 8.88 and 8.03 ppm. These signal were broad, while the signals of 3a stayed sharp, suggesting that the Pt(II) species interacts with the high MW proteins in the cell culture medium (probably BSA), while the starting Pt(IV) dimer does not. Integration of the phenanthroline peaks of Pt(IV)-phen and Pt56MeSS indicated that after 1 day about 22% of the Pt(IV)-phen moiety was reduced and after 2 days 39% and after 3 days 58%. It is interesting to note that compound 3a is more stable in buffer than in cell culture medium since the monomeric compounds Pt56MeSS(OAc)₂ and Pt56MeSS(PhB)₂ were unstable in buffer but were stable in human serum. In the range 7.43-7.25 ppm the signals of free phenylbutyrate were detected, overlapping with the amidic signal of 3a and 4. Finally, a signal belonging to free DCA was detected at 6.06 ppm. The following day, the signals belonging to [Pt(DACH)(56Phen)]²⁺ and free PhBu, increased in intensity, suggesting that the reduction of the Pt metal center of 3a bond to the phenanthroline was continuing. On the other hand, almost no more coordinated DCA was detected, while free DCA grew in intensity, suggesting that the hydrolysis of the cisplatin-like part of 3a was almost complete. The same trend was observed on the third day.

Compared to the pervious experiment, in which the stability of 3a was tested in phosphate buffer, the experiment carried out in cell culture medium showed the same degradation pathway, in which the Pt metal center bond to the phenanthroline ligand undergoes reduction, while the Pt metal center that has DCA as axial ligand undergoes hydrolysis. The process seems to occur faster in cell culture medium.

Study of Reduction in Buffer in the Presence of Ascorbic Acid.

In order to assay which of the two Pt metal centers of 3a is more prone to be reduced, we added 1 Eq of ascorbic acid to a solution of 3a in phosphate buffer. We added 1 Eq of ascorbic instead of 2 Eq that would be needed for the reduction of both Pt cores, to assay if one of the two is preferably reduced over the other. Finally, we carried out the reaction at RT and not at 37° C. to avoid a fast reduction process that would have made more complicated its study.

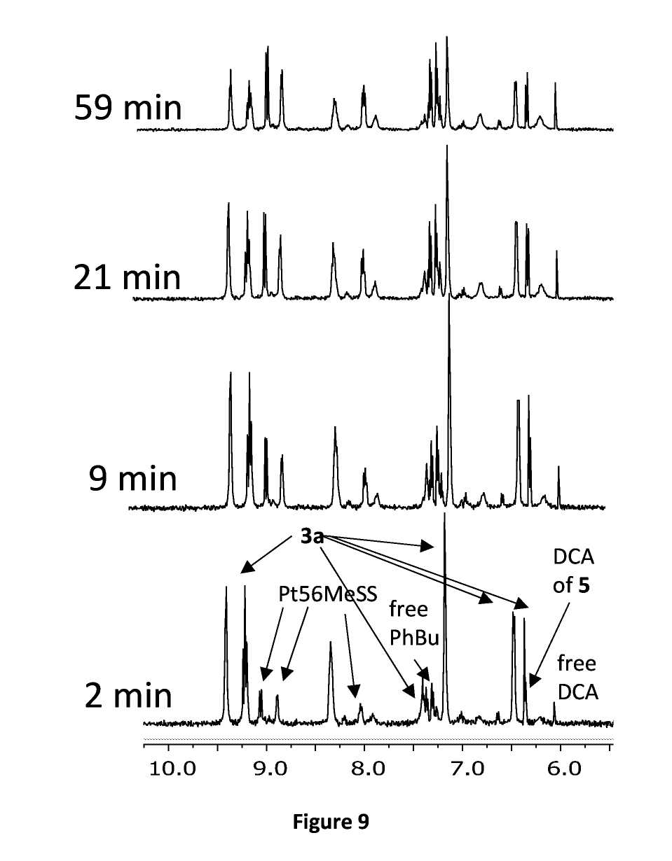

The reaction was monitored by ¹H NMR spectroscopy (FIG. 9). Already after 2 min, time needed to prepare the sample and start the NMR experiment, besides the signals belonging to 3a, previously discussed, it was possible to note the signal belonging to [Pt(DACH)(56Phen)]²⁺, free phenyl butyric acid and a signal at 6.34 ppm, consistent with a DCA moiety coordinated to a Pt(IV) metal center. These signals indicated the reduction of the phenanthroline-bonded Pt unit of 3a to [Pt(DACH)(56Phen)]²⁺, and the release of the axial ligands, butyric acid and c,c,t-[Pt(NH₃)₂(DCA)(OOCCH₂CH₂CH₂NHCOCH₂CH₂COOH)] (5). We were not able to isolate the latter species. The other metal center of 3a underwent reduction as well, as indicated by the presence of free DCA as product of reductive elimination, but at a lower extent, as indicated by the lower intensity of the signal of free DCA. The reaction was monitored for about 2 hours. In FIG. 9 are reported the spectra recorded after 9, 21 and 59 min. In the second hour, no changes were detected. The same trend already noticed after the first two minutes was maintained over the time: a faster reduction of the phenanthroline-bonded Pt unit, and a slower reduction of the other metal center of 3a.

This behavior is in agreement with the CV measurements, that showed a reduction potential more negative for the cisplatin-derived Pt metal center of 3a, that is, so, less prone to undergo reduction.

The two dimers, 3a and 3b, as well as compounds 1a and 2a were screened against six human cancer cell lines representative of lung (H157), colon (HCT-15), cervical (A431), ovarian (2008) and pancreatic (PSN1) cancers, along with melanoma (A375). Cytotoxicity was evaluated by means of the MTT test after 72 h of treatment and the in vitro antitumor potential of newly synthesized derivatives was compared to that of the reference metallo-drugs CDDP and OXP. The results, expressed as IC₅₀ values calculated from dose-survival curves, are reported in Table 5. Compound 1a showed a pattern of cytotoxicity similar to that of CDDP against colon cancer cells and melanoma cells whereas it was more effective than the reference metallodrug against pancreatic cancer cells PSN1. Compound 2a proved to be more effective than cisplatin against colon and pancreatic cancer cells as well as melanoma cells whereas it retains an in vitro antitumor potential lower than that of cisplatin against cervical cancer cells A431. On the other hand, the “quadrupole action” complex (3a) was more potent than CDDP and OXP in all tested cancer cell lines and its cytotoxicity resembles that of the parental compound [Pt(DACH)(56Phen)]. Interestingly, compound 3a was significantly (on average about 12 times) more potent than the “dual action” compound 3b. This clearly attests to the importance of tethering bioactive ligands such as DCA and PhB to the axial position in order to attain superior in vitro antitumor potential. It is interesting to note that 3a was up to 200 and 92 times more effective than CDDP and OXP, respectively, against PSN1 cells, that a notorious highly aggressive p53 and KRAS mutated pancreatic cancer cells.

TABLE 5
Cells (3 − 8 × 10⁴ mL⁻¹) were treated for 72 h with increasing
concentrations of tested compounds. The cytotoxicity was assessed by the MTT test.
IC₅₀ values were calculated by a four parameter logistic model (P < 0.05).

| | IC₅₀ (μM) ± S.D. | | | | | |
|---|---|---|---|---|---|---|
| Compound | 2008 | A431 | H157 | HCT-15 | A375 | PSN1 |
| 1a | — | 4.98 ± 1.63 | — | 12.45 ± 3.58 | 2.81 ± 0.21 | 4.74 ± 2.87 |
| 2a | — | 4.15 ± 1.12 | — | 6.25 ± 3.08 | 1.57 ± 0.16 | 3.23 ± 1.12 |
| 3a | 0.79 ± 0.09 | 0.20 ± 0.09 | 0.95 ± 0.08 | 0.36 ± 0.08 | 0.08 ± 0.01 | 0.09 ± 0.02 |
| 3b | 6.04 ± 1.08 | 5.25 ± 0.41 | 8.25 ± 1.21 | 1.89 ± 0.69 | 1.05 ± 0.18 | 6.12 ± 1.11 |
| [Pt(DACH) | 0.74 ± 0.12 | 0.28 ± 0.08 | 0.16 ± 0.01 | 0.69 ± 0.11 | 1.12 ± 0.42 | 0.25 ± 0.08 |
| (56Phen)] | | | | | | |
| CDDP | 2.22 ± 1.02 | 1.65 ± 0.51 | 3.28 ± 2.38 | 15.28 ± 2.63 | 3.11 ± 0.98 | 18.25 ± 3.11 |
| OXP | 1.53 ± 0.88 | 3.06 ± 0.88 | 5.99 ± 1.85 | 1.15 ± 0.43 | 6.30 ± 2.01 | 8.25 ± 3.42 |

SD = standard deviation.

In Vitro Antitumor Activity on 3D Human Cancer Cell Cultures

Cytotoxicity results obtained by MTT test showed that compounds 3a and 3b elicited a significant antiproliferative activity against cancer cells derived from solid tumors. However, in two-dimensional (2D) monolayer cell cultures cellular activities are often altered and their typical in vivo functions resulted to be lost. As a result, the conventional 2D cell culture provides limited predictive capacity for drug testing.

As an attractive alternative, 3D cell cultures have proven to be a physiologic mimic of the in vivo tissue because they produce a similar cellular microenvironment. Actually, on a three-dimensional architectural organization, tumor cells are not uniformly exposed to nutrients or oxygen in vitro, thus closely mimicking the organization of human tumors. Thus, significant differences were described in terms of sensitivity to drugs among 2D and 3D cultures due to fundamental differences in terms of growth, migration, morphology and gene expression.

In order to improve the predictive capacity of the in vitro assays, we tested the activity in three three-dimensional (3D) cell cultures; human colon cancer cells, HCT-15, and human pancreatic cancer cells, KRAS wild type BxPC3 cells KRAS mutated PSN-1 cells. The cancer spheroids were treated with 3a and 3b for 72 h and the cell viability was assessed by means of the APH assay (Table 6). For comparison purposes, the cytotoxicities of CDDP and OXP were evaluated under the same experimental conditions.

In all human cancer spheroid models, derivatives 3a and 3b yielded IC₅₀ values lower than that showed by CDDP. In the HCT-15 3D model, 3a was about 2 times more effective than CDDP and its antitumor potential was almost comparable to that of OXP. Notably, against PSN-1 pancreatic cancer cells the activity of 3a exceeded that of the reference drugs by a factor of about 6. These results, besides clearly indicating a significant antitumor potential for compound 3a, suggest its preferential activity toward highly aggressive KRAS mutated pancreatic cancer cells PSN-1.

TABLE 6
Spheroids (2.5 · 10³ cells/well) were treated for
72 h with increasing concentrations of tested compounds.

| | IC₅₀ (μM) ± S.D. | | |
|---|---|---|---|
| Compound | HCT-15 | BxPC3 | PSN-1 |
| 3a | 39.96 ± 4.58 | 59.15 ± 3.84 | 10.29 ± 4.85 |
| 3b | 75.85 ± 4.52 | 81.28 ± 6.25 | 60.45 ± 5.76 |
| [Pt(DACH)(56Phen)] | 60.19 ± 2.92 | 100.5 ± 12.13 | 55.53 ± 9.75 |
| COOP | 89.20 ± 3.44 | 69.58 ± 8.21 | 59.89 ± 12.92 |
| OXP | 36.45 ± 3.12 | 75.21 ± 6.91 | 59.68 ± 8.52 |

The growth inhibitory effect was evaluated by means of APH test.
IC₅₀ values were calculated from the dose-survival curves by four parameter logistic model (P < 0.05).
SD = standard deviation.

Cytotoxicity Against Non-Cancer Cells

The cytotoxic activity of derivatives 3a and 3b was also evaluated against non-tumor cells, namely human embryonic kidney HEK293 cells (Table 7). Both compounds 3a and 3b were more cytotoxic than CDDP. However, the selectivity index (SI=ratio between average IC₅₀ of non-tumor cells and IC₅₀ of malignant cells) calculated for complex 3b was lower than that elicited by CDDP whereas the SI for compound 3a was about 2 times better than that of cisplatin, thus attesting the preferential cytotoxicity of 3a towards neoplastic cells.

TABLE 7
Cells (5 × 10⁴ mL⁻¹) were treated for 72 h with
increasing concentrations of tested compounds.

IC₅₀ (μM) ± S.D.

| Compound | HEK293 | SI |
|---|---|---|
| 3a | 2.25 ± 0.29 | 5.5 |
| 3b | 7.80 ± 3.36 | 1.7 |
| CDDP | 19.56 ± 2.95 | 2.7 |
| Pt56MeSS | 1.12 ± 0.87 | 0.9 |

Cytotoxicity was assessed by MTT test.
IC₅₀ values were calculated by four parameter logistic model (p < 0.05).
S.D. = standard deviation.
CDDP = cisplatin.

Cell Accumulation and Distribution

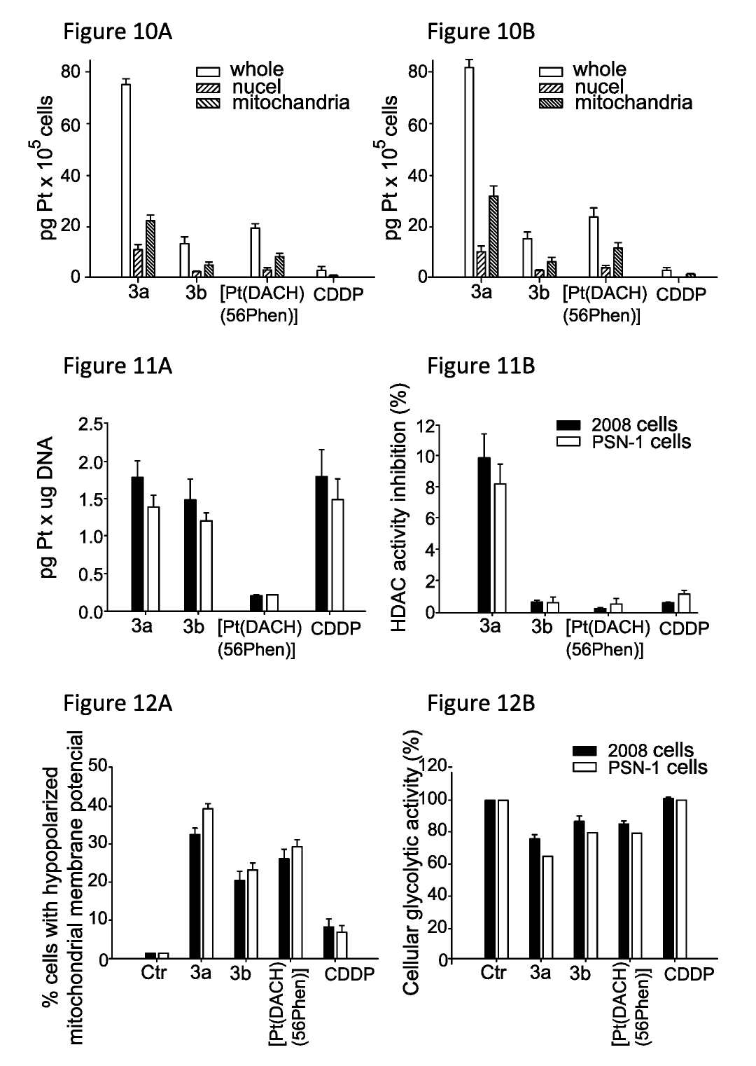

With the aim of identifying a possible correlation between cytotoxic activity and cellular accumulation, the cellular Pt content and distribution were measured in human ovarian 2008 and pancreatic PSN-1 cancer cells treated for 24 h with equimolar concentrations (0.1 μM) of the platinum compounds. The cellular Pt levels in cellular sub-fractions were quantified by means of GF-AAS analysis, and the results expressed as pg Pt per 10⁵ cells, are summarized in FIG. 10 (A: 2008 cells, B: PSN-1 cells).

Although the IC₅₀ values of 3a and Pt56MeSS are practically identical in the ovarian cancer cell line 2008, there is nearly a fourfold difference in their cellular association and a twofold difference in their accumulation in their nuclie and mitochondria, in favor of 3a. Also, cisplatin is nearly 3 fold more potent than 3b against 2008 cells despite it much lower levels of cell association and nuclear and mitochondrial accumulation. The cell association, nuclear and mitochondrial accumulation in the PSN1 cells seem to correlate qualitatively with cytotoxicity.

In both cell lines, the lowest level of platinum associated with the cells was observed after exposure to cisplatin and, remarkably, the replacement of axial acetato groups in 3a by PhB and DCA (3b) resulted in a very significant enhancement of the level of platinum associated with the cells.

Concerning cellular distribution of Pt, 3a and 3b as well as the reference [Pt(DACH)(56Phen)] were able to highly accumulate into the mitochondrial fraction. This is probably due to the reduction of 3a and 3b in the cells that releases that cationic and lipophilic Pt56MeSS that accumulates in the mitochondria. Maybe another way to look at this is the % in the nucleus compared to the whole cell association. It seems to me that in 2008 there is more cisplatin in the nucleus (compared to mitochondria) and the other way around in PSN1.

HDAC Inhibition and DNA Platination

The newly prepared 3a complex described here was designed as quadruple action prodrug able to intracellularly release four bioactive compounds with different cellular targets and mechanisms of action. In particular, the release of PhB moiety inside the cells should grant the inhibit HDAC activity, thus allowing for a more efficient DNA platination.

Thus, we measured both the levels of DNA platination and the ability of these pro-drugs to inhibit HDAC activity in cancer cells. 2008 and PSN-1 cells were incubated for 24 h with cytotoxic IC₅₀ concentrations of 3a, 3b, CDDP or [Pt(DACH)(56Phen)], and their DNA-platination ability as well as the HDAC inhibitory activity was determined (FIGS. 11A and B).

DNA-platination studies (FIG. 11A) confirmed that [Pt(DACH)(56Phen)] does not exert its antitumor activity by covalently interacting with DNA. 2008 and PSN-1 cells treated with compound 3a and 3b showed DNA-platination levels resembling, or slightly lower in the case of 3b, that obtained with reference compound CDDP. This is noteworthy as it indicates that the differences in the IC₅₀ values of 3a, 3b and cisplatin in 2008 (0.79, 6.04 and 2.22 μM) and PSN1 (0.09, 6.12 and 18.25 μM) do not stem merely from different levels of DNA platination and other factor significantly affect their cytotoxic potential.

Concerning HDAC inhibitory activity, as expected CDDP and the Pt(IV) complex 3b were not effective in modifying HDAC activity (FIG. 11B). Conversely, the PhB bearing compound 3a hampered by about 8 and 10% HDAC activity in 2008 and PSN-1 cells, respectively.

DCA Activity (Similar to Lippard Paper)

It has been previously demonstrated that DCA inhibits the activity of pyruvate dehydrogenase kinase (PDK), thereby shifting cellular metabolism from glycolysis to glucose oxidation, altering mitochondrial homeostasis and ultimately leading to cell death through apoptosis.

Based on the above findings, we investigated the effects determined by derivatives 3a and 3b on 2008 and PSN-1 cancer cell aerobic glycolysis and mitochondria function.

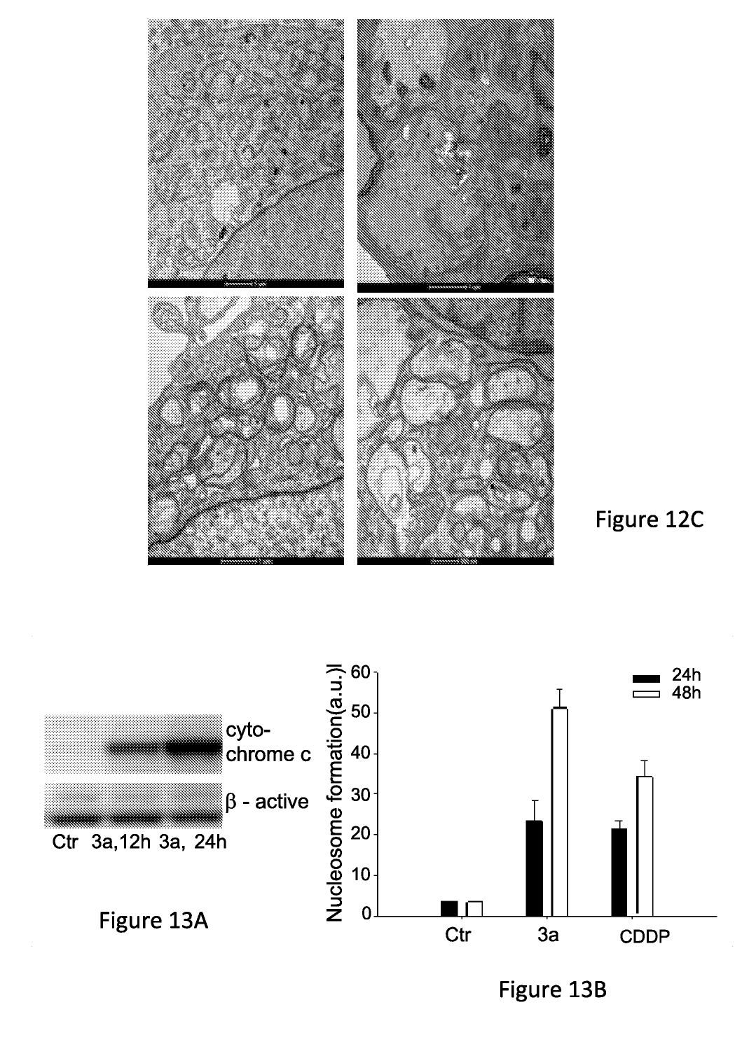

Treatment of 2008 cells with IC₅₀ concentrations of both 3a and 3b for 24 h resulted in a reduction (about 25 and 14% for 3a and 3b, respectively) of glycolysis processes compared with untreated control cells (FIG. 12A). However, these effects were magnified in PSN-1 cells, having reduction of cellular glycolytic activity of about 36 and 20% for 3a and 3b, respectively. These results, besides clearly attesting to the fact that 3a, that releases DCA inside the cell, acts by significantly shifting cellular metabolism toward oxidative phosphorylation, also revealed compound 3b, that lacks DCA, can hamper cancer cell glycolysis. This latter result could be explained tacking into consideration that the reference compound [Pt(DACH)(56Phen)] itself, that is also released upon reduction of 3a and 3b, is effective in hampering cellular glycolysis in both tested cancer cell lines. The difference between 3a and 3b might be attributed to the added effect of DCA.

In addition, it is interesting to note that treatment of 2008 and PSN-1 cells with 3a and 3b determined a significant time-dependent increase in the number of cells with depolarized mitochondria (data not shown). In particular, after 12 h of incubation, 3a elicited an increase of about 39% of PSN-1 cells with depolarized mitochondrial potential FIG. 12B). Again, the reference compound [Pt(DACH)(56Phen)] was effective in hampering cellular mitochondrial membrane potential in both tested cancer cell lines.

Morphological analyses by TEM of PSN-1 cells treated for 28 h with both Pt(IV) derivatives showed disrupted cristae and significant increase in volume (swelling) with respect to control cells (FIG. 7C). Besides expected for derivative 3a bearing the DCA moiety, these results highlighted a possible “antimitochondrial” effect also for compound 3b.

Cytochrome c Release and Apoptosis Induction

It has been previously shown that DCA facilitates translocation of proapoptotic mediators, like cytochrome c, thus stimulating apoptosis. In addition, DNA-damaging agents as well as HDAC inhibitors are known to drive cancer cells to commit suicide by apoptosis. Therefore, in order to highlight the nature of the cell death determined by 3a, cytochrome c release and classical apoptosis markers were monitored.

By monitoring cytochrome c release in PSN-1 cells after 12 and 24 h of treatment with IC₅₀ of compound 3a, we observed that the compound caused a substantial time-dependent release of cytochrome c from the mitochondria to the cytosol (FIG. 13A), thus the suggesting the induction of a mitochondrial-mediated pathway of cell death. Furthermore, nuclear DNA fragmentation and apoptosome formation were substantially increased in PSN-1 cells treated for 24 or 48 with 3a (FIG. 13B). After 48 h of treatment a roughly 20-fold increase in histone-DNA formation was detected with 3a with respect to controls.

In addition, Hoechst staining of PSN-1 cells after 72 h incubation with 3a clearly indicated the occurrence of nuclear condensation (pyknosis) and blebbing (FIG. 13C).

Pancreatic cancer is one of the most aggressive tumors with a dramatically poor prognosis and an incidence rate equaling mortality rate. Current evidence has reveal that pancreatic cancer cells possess an altered metabolic/energetic redox regulation primarily due to the oncogenic Kras mutation, which enhances glucose and glutamine metabolisms by upregulating the expression of a variety of key enzymes involved in this peculiar pathways. These peculiar characteristic make this cells particularly sensitive to cellular redox alterations, highlighting the therapeutic potential of targeting pancreatic cells by compounds able to shift the redox metabolism toward an oxidative one.

1. A compound comprising a platinum atom associated to one or more phenyl butyrate ligand, the compound optionally comprising one or more therapeutically active anticancer moiety.

2. The compound according to claim 1, having at least two phenylbutyrate ligands.

3. (canceled)

4. The compound according to claim 1, comprising at least one ligand selected from ligands herein designated L1 through L47 or L48 through L63.

5. (canceled)

6. (canceled)

7. The compound according to claim 1, being of Formula (I): 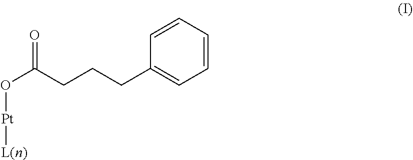
wherein
L is a ligand moiety selected from substituted or unsubstituted alkyl, substituted or unsubstituted alkenyl, substituted or unsubstituted alkynyl, substituted or unsubstituted cycloalkyl, substituted or unsubstituted cycloalkenyl, substituted or unsubstituted cycloalkynyl, substituted or unsubstituted aryl, substituted or unsubstituted heteroaryl, substituted or unsubstituted heterocyclyl, halogen, substituted or unsubstituted —NR₁R₂, substituted or unsubstituted —OR₃, substituted or unsubstituted —SR₄, substituted or unsubstituted —S(O)R₅, substituted or unsubstituted alkylene-COOH, substituted or unsubstituted ester, —OH, —SH, and —NH; or
L is a linker group bonded to at least one other Pt atom;
wherein each of said R₁, R₂, R₃, R₄ and R₅, independently of the other, is selected from substituted or unsubstituted alkyl, substituted or unsubstituted alkenyl, substituted or unsubstituted alkynyl, substituted or unsubstituted cycloalkyl, substituted or unsubstituted cycloalkenyl, substituted or unsubstituted cycloalkynyl, substituted or unsubstituted aryl, substituted or unsubstituted heteroaryl, substituted or unsubstituted heterocyclyl;
and wherein n is the number of ligand moieties, being 1, 2, 3, 4, or 5.

8. The compound according to claim 7, wherein ligand L is selected amongst ligands herein designated L1 through L63.

9. The compound according to claim 7, being of Formula (Ia):

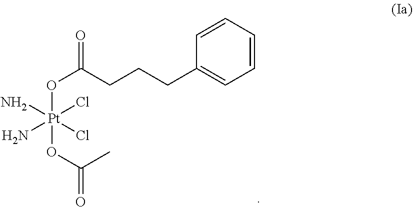

10. The compound according to claim 7, having a Formula selected amongst Formulae (II) through (XXXI): 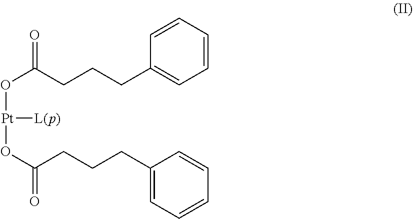
wherein L is as defined in claim 7; and p is 1, 2, 3, or 4, 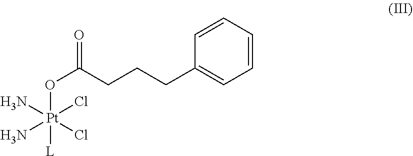
wherein L is a ligand different than phenylbutyrate; optionally selected from therapeutically active anticancer moieties 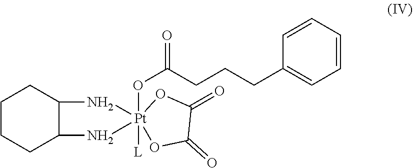
wherein L is a ligand different than phenylbutyrate; optionally selected from therapeutically active anticancer moieties 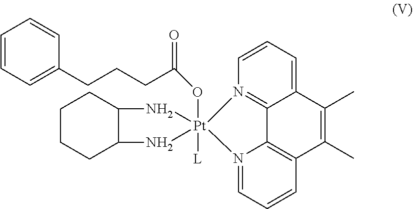
wherein L is a ligand different than phenylbutyrate; optionally selected from therapeutically active anticancer moieties 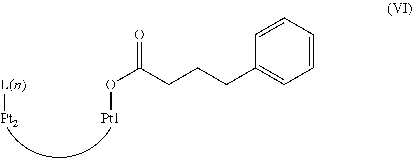
wherein L and n are each as defined in claim 7, and wherein the curved line is a linker group 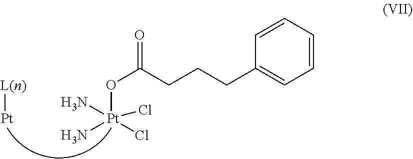
wherein L and n are each as defined in claim 7, and wherein the curved line is a linker group 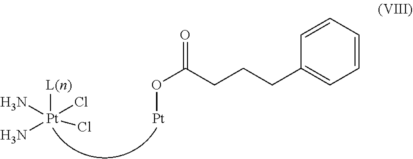
wherein L and n are each as defined in claim 7, and wherein the curved line is a linker group 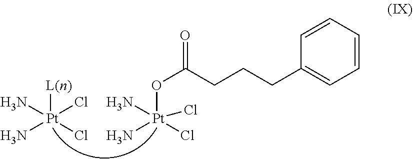
wherein L and n are each as defined in claim 7, and wherein the curved line is a linker group 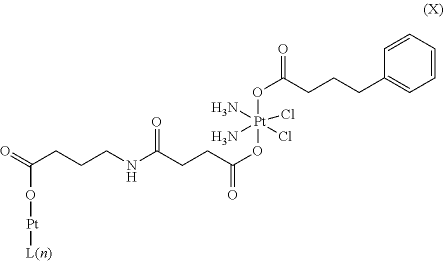
wherein L and n are each as defined in claim 7 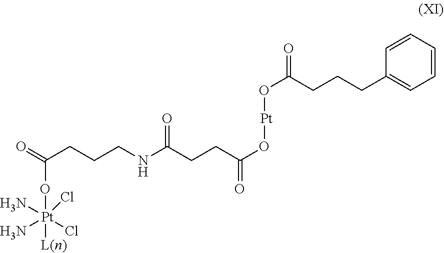
wherein L and n are each as defined in claim 7 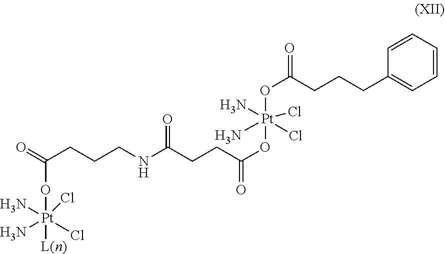
wherein L and n are each as defined in claim 7 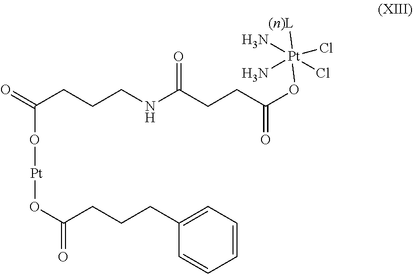
wherein each of L and n is as defined in claim 7 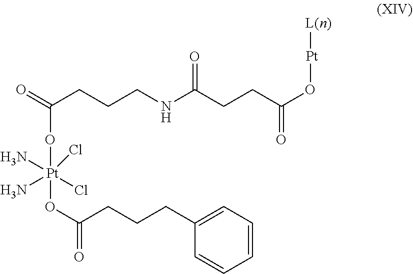
wherein each of L and n is as defined in claim 7 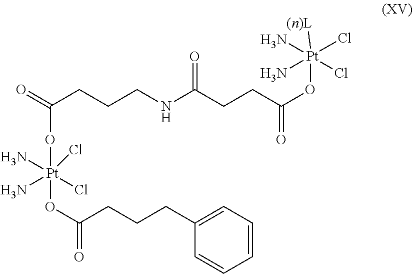
wherein each of L and n is as defined in claim 7 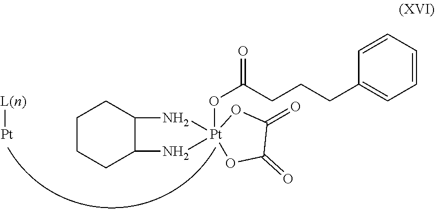
wherein L and n are each as defined in claim 7, and wherein the curved line is a linker group 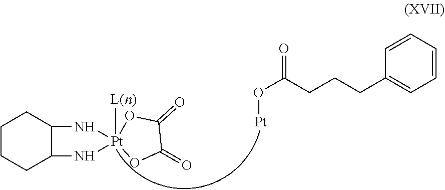
wherein L and n are each as defined in claim 7, and wherein the curved line is a linker group 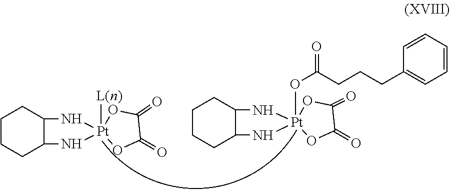
wherein L and n are each as defined in claim 7, and wherein the curved line is a linker group 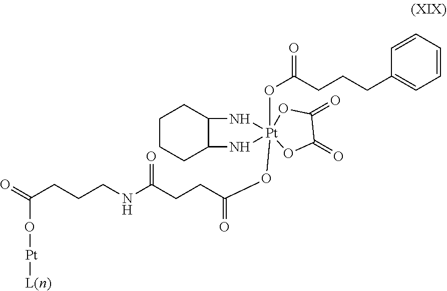
wherein each of L and n are as defined in claim 7 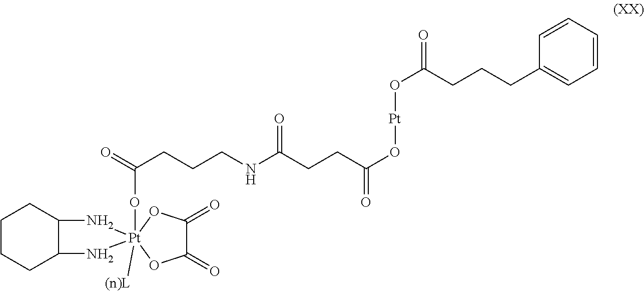
wherein each of L and n are as defined in claim 7 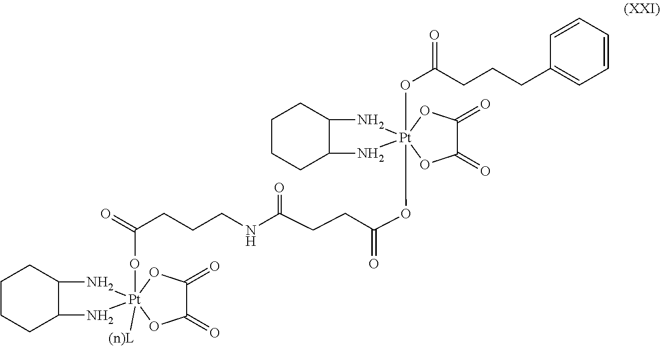
wherein each of L and n are as defined in claim 7 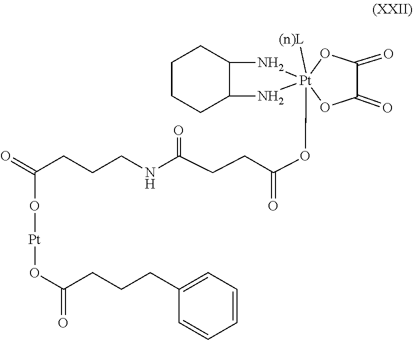
wherein each of L and n are as defined in claim 7 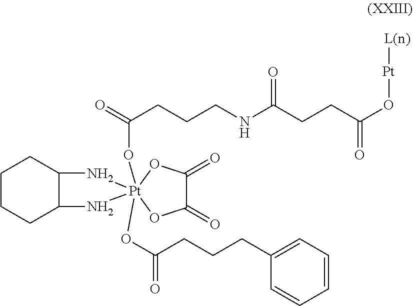
wherein each of L and n are as defined in claim 7 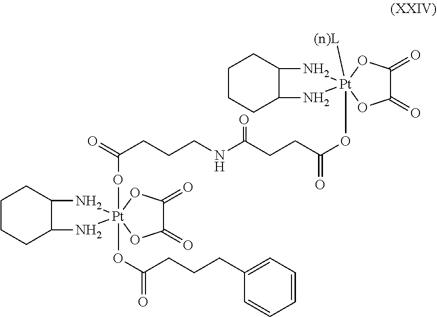
wherein each of L and n is as defined in claim 7 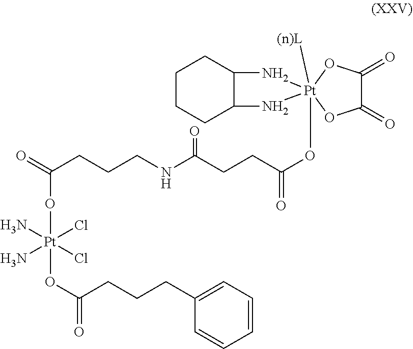
wherein each of L and n are as defined in claim 7 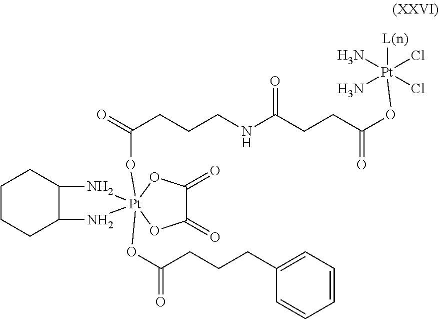
wherein each of L and n is as defined in claim 7 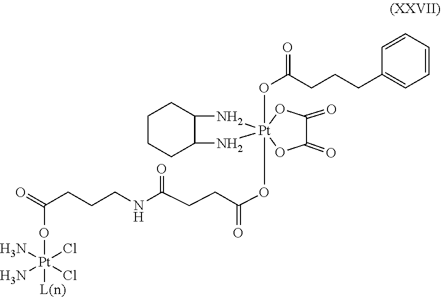
wherein each of L and n are as defined in claim 7 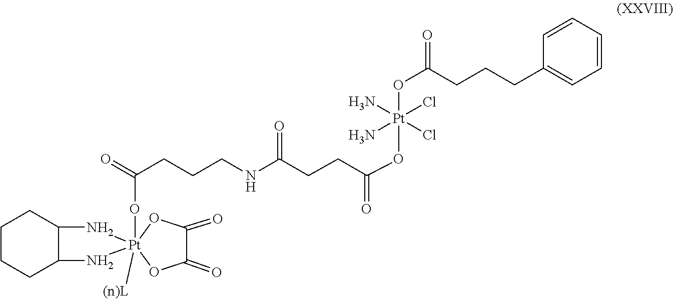
wherein each of L and n is as defined in claim 7 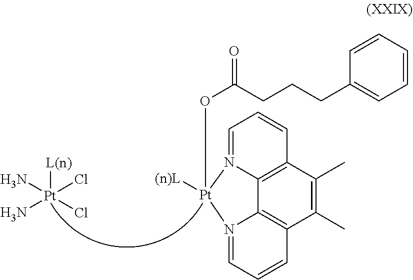
wherein L and n are each as defined in claim 7, and wherein the curved line is a linker group 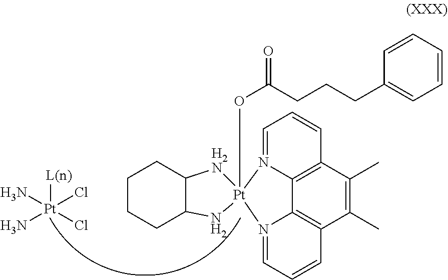
wherein L and n are each as defined in claim 7, and wherein the curved line is a linker group; and 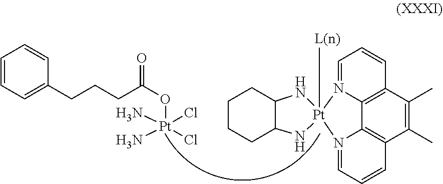
wherein each of L and n is as defined in claim 7, and wherein the curved line is a linker group.

11. The compound according to claim 10, selected from the group consisting of: 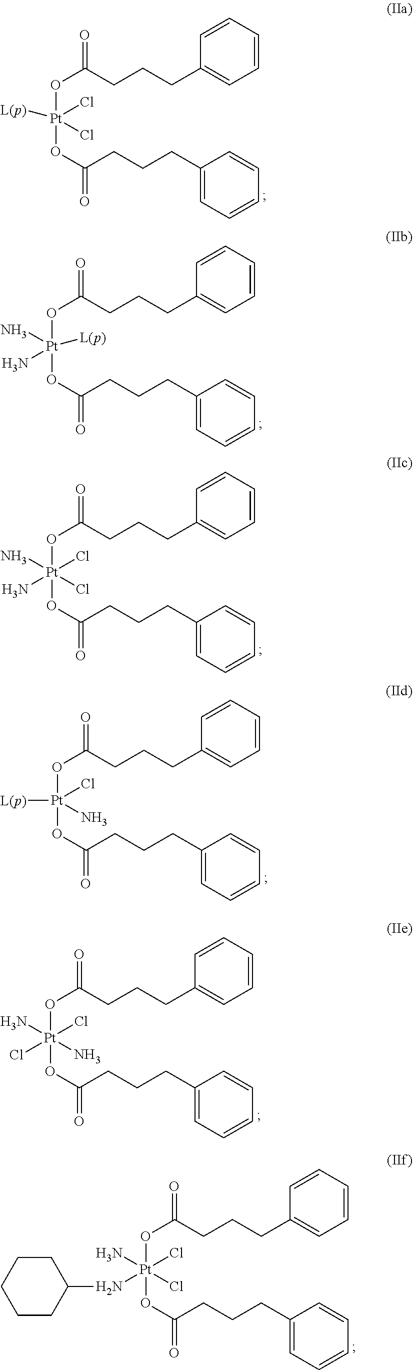 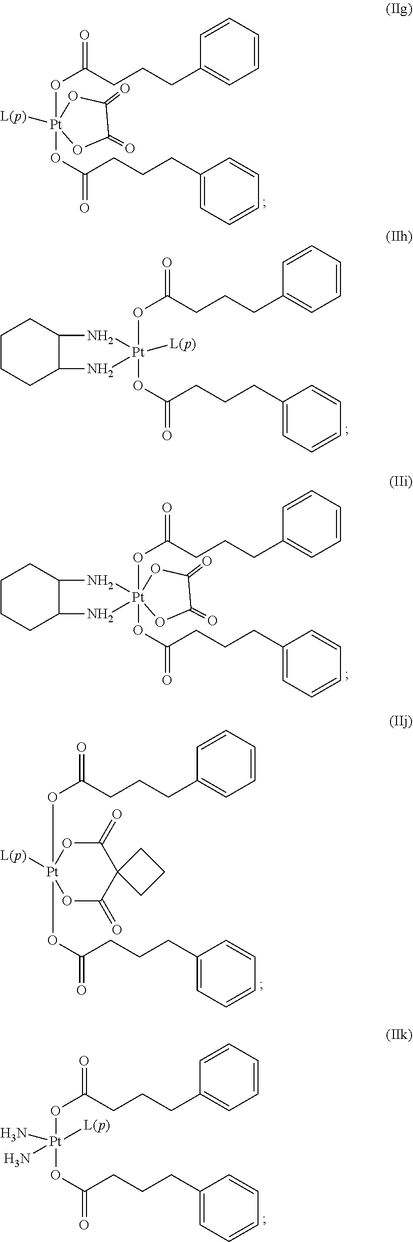 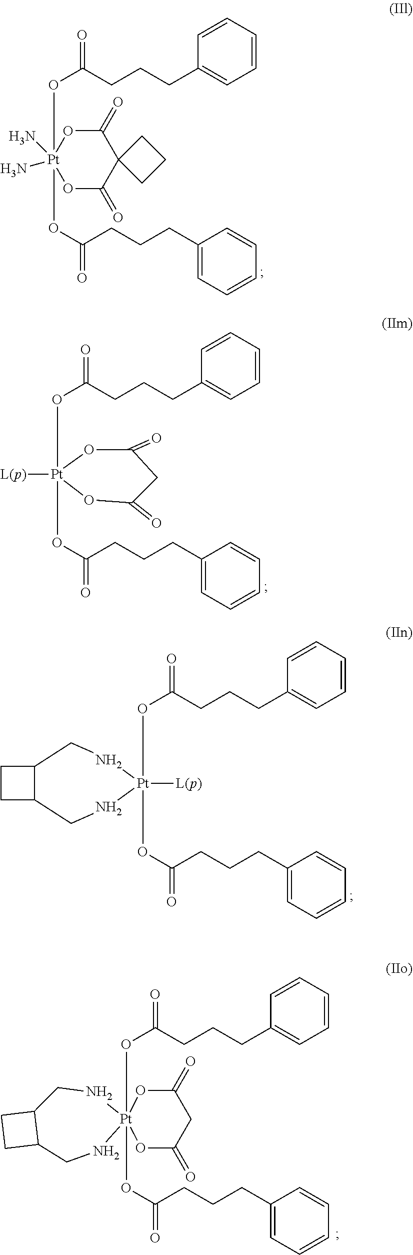 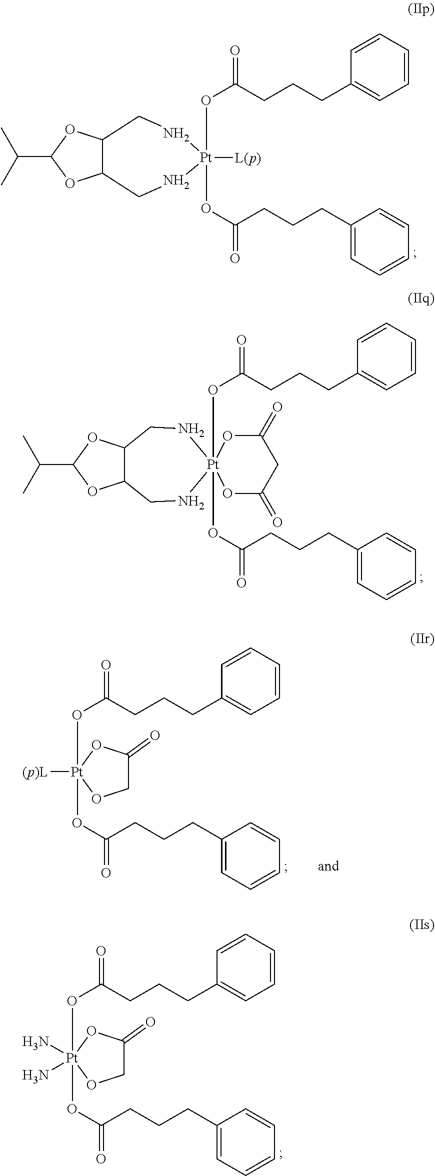
wherein L and p are each as defined in claim 10.

12.-30. (canceled)

31. The compound according to claim 10, selected from the group consisting of:

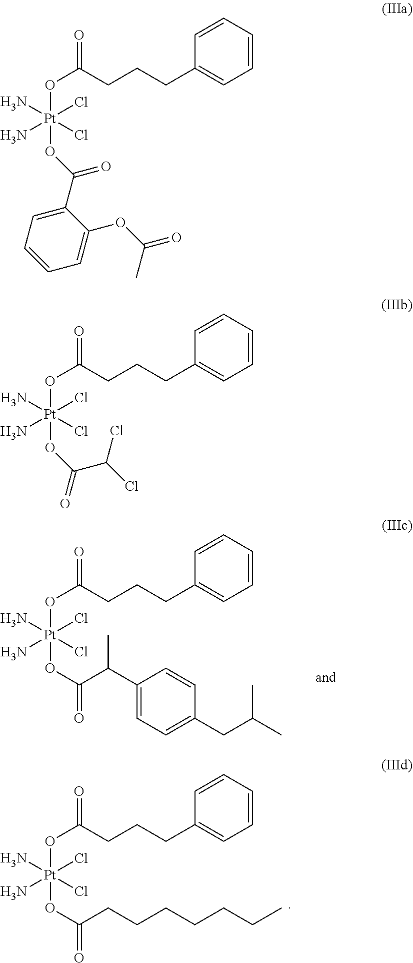

32. (canceled)

33. The compound according to claim 10, selected from the group consisting of:

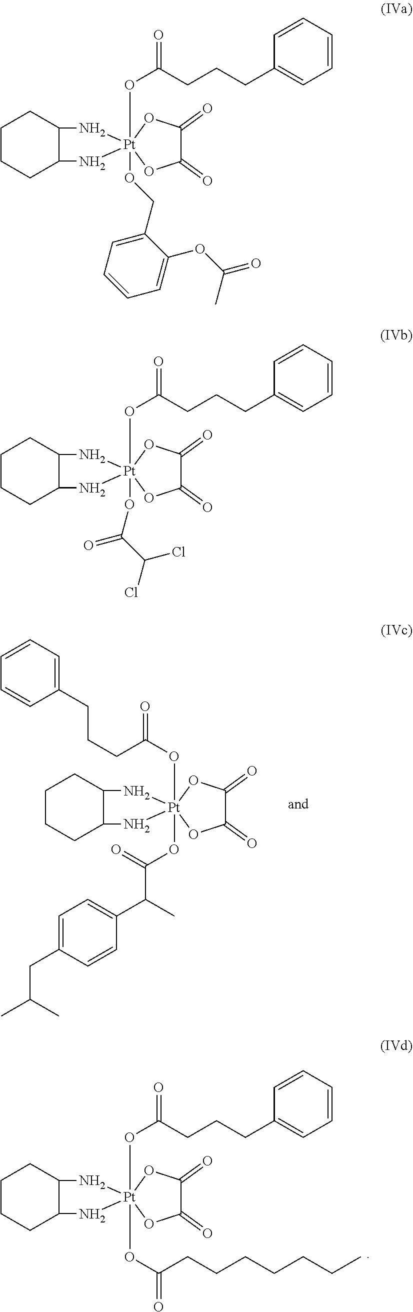

34. (canceled)

35. The compound according to claim 10, selected from the group consisting of:

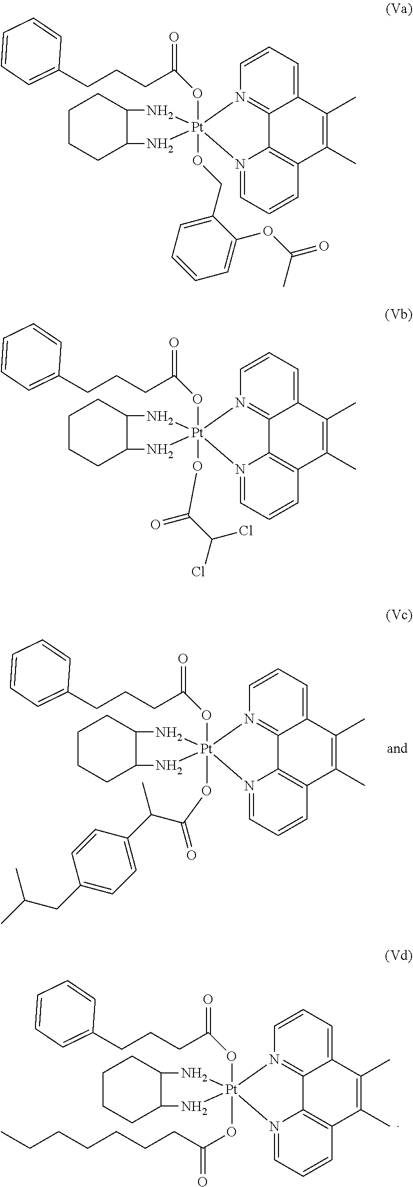

36.-51. (canceled)

52. The compound according to claim 1, wherein the therapeutically active anticancer moiety is selected from the group consisting of pyruvate dehydrogenase kinase (PDK) inhibitors, histone deacetylase (HDAC) inhibitors, DNA methylation enhancers, COX inhibitors, PARP inhibitors, DNA repair inhibitors and cancer targeting moieties.

53.-57. (canceled)

58. A compound selected from: 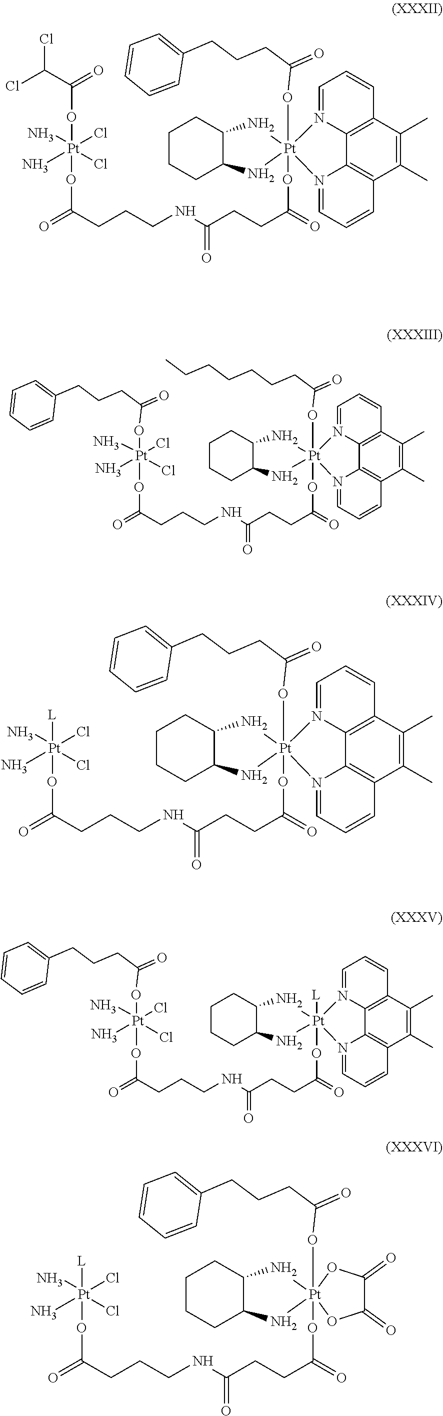 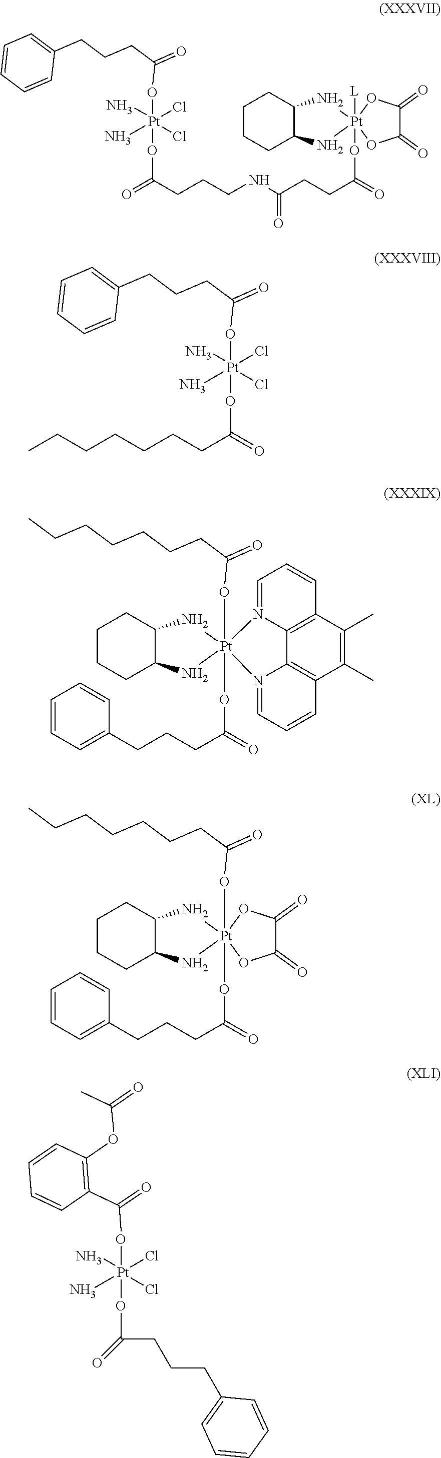 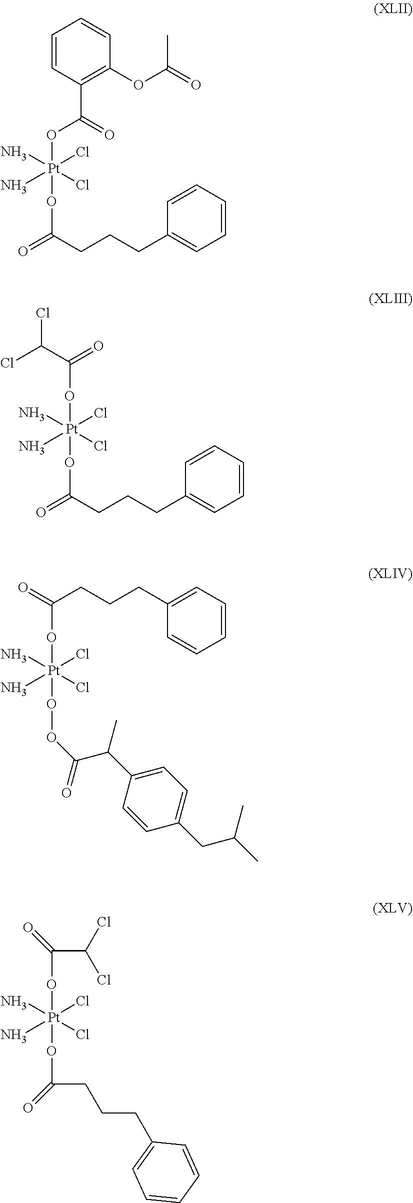 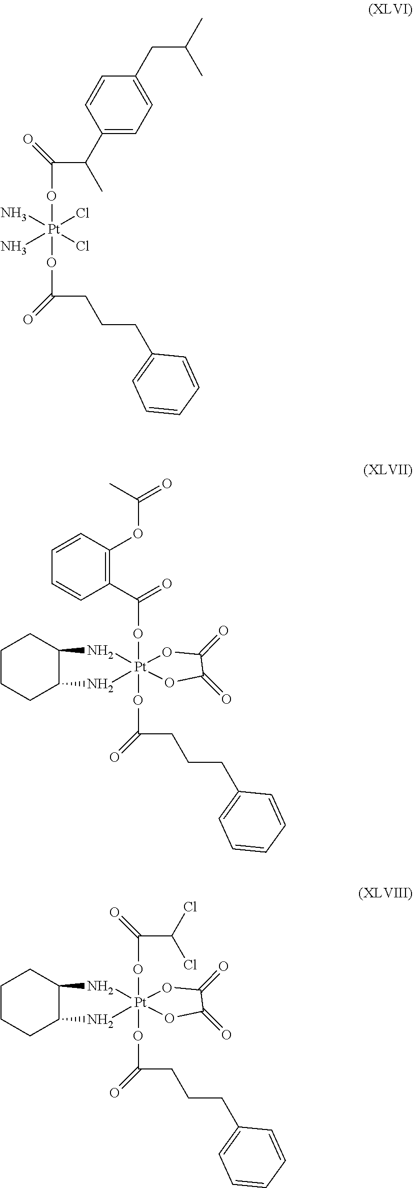 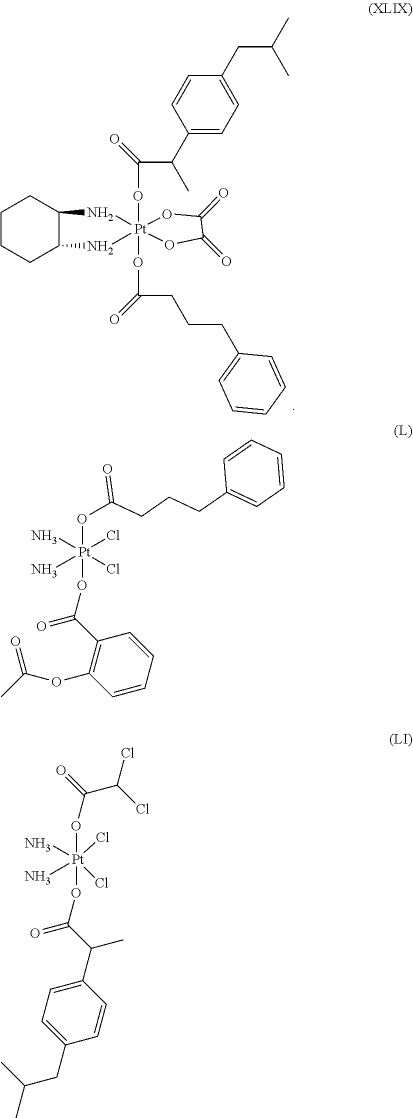 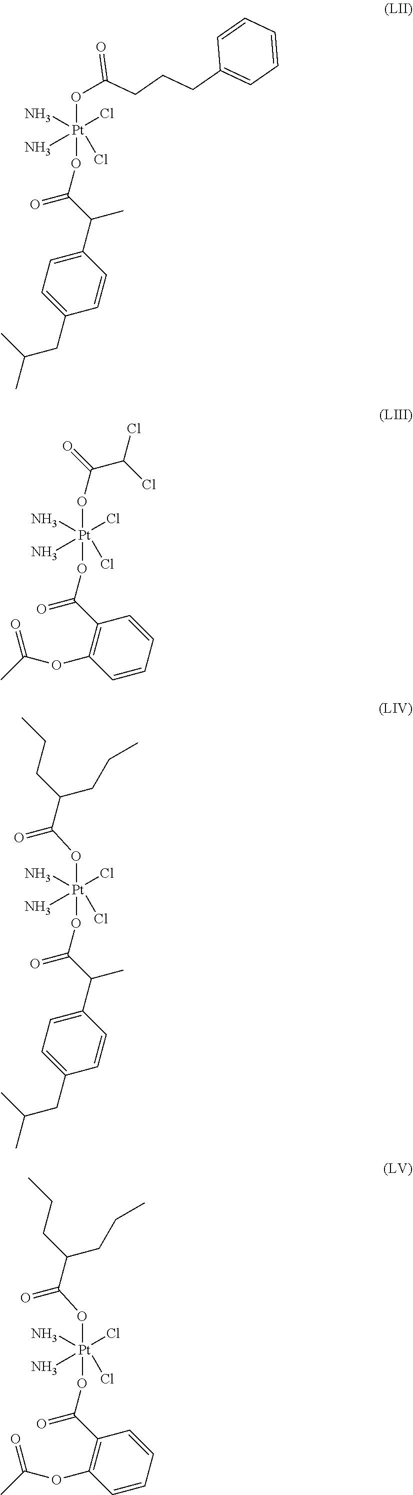 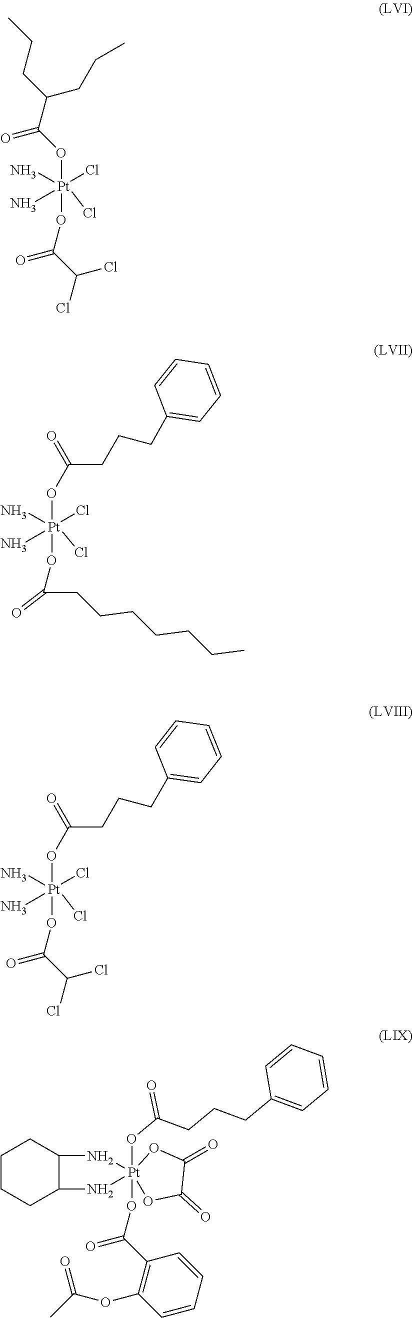 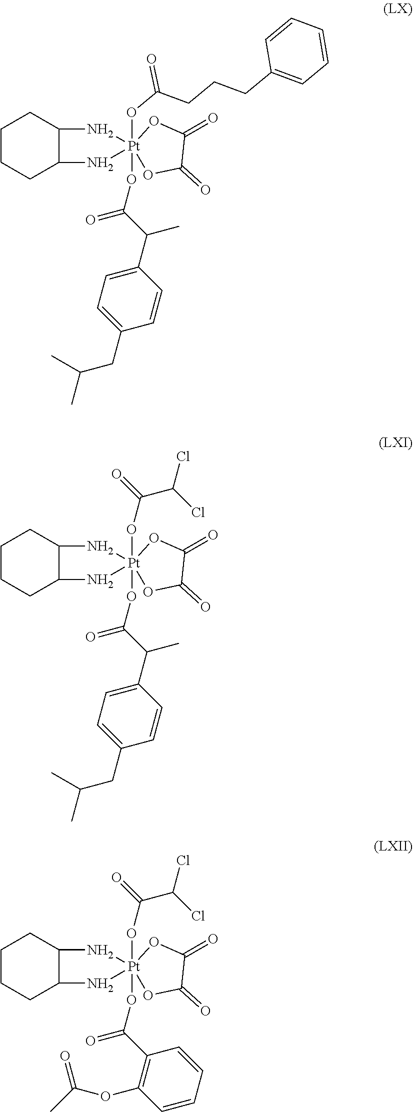 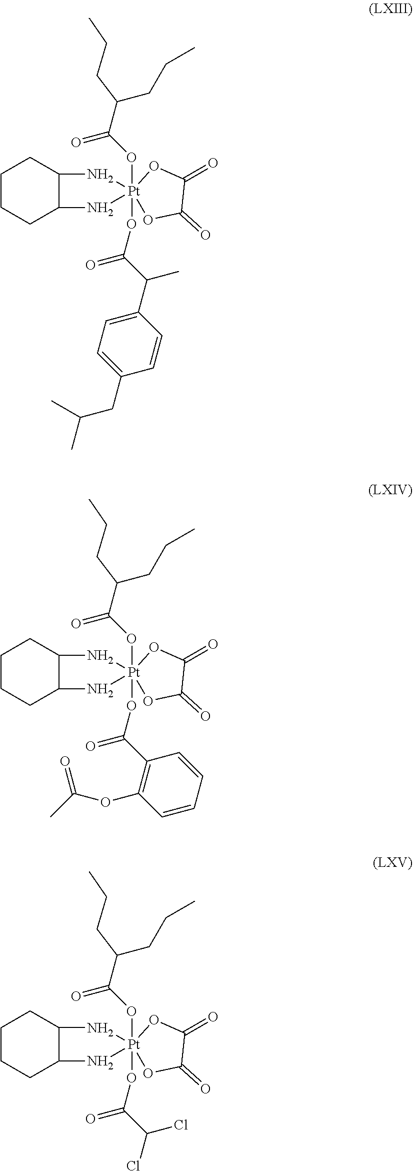 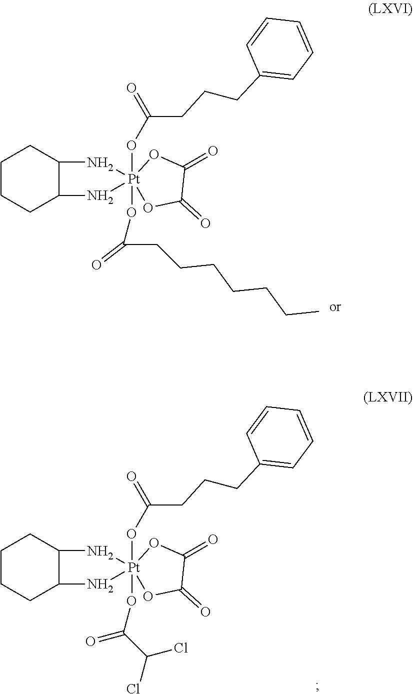
wherein L is a therapeutically active anticancer moiety selected from the group consisting of pyruvate dehydrogenase kinase (PDK) inhibitors, histone deacetylase (HDAC) inhibitors, DNA methylation enhancers, COX inhibitors, PARP inhibitors, DNA repair inhibitors and cancer targeting moieties,
wherein each of the therapeutically active anticancer moiety may be bonded via a —O—C(═O)— group.

59. A composition comprising a compound according to claim 1.

60.-62. (canceled)

63. A method for the treatment of a proliferative disorder, the method comprising administering an effective amount of an agent according to claim 1 to a subject suffering from said disorder.

64. (canceled)
